(19) United States
(12) Patent Application Publication
(10) Pub. No.: US 2010/0316991 A1
(43) Pub. Date: Dec. 16, 2010

(54) HUMAN PARAINFLUENZA VIRUS TYPE 3 EXPRESSING THE ENHANCED GREEN FLUORESCENT PROTEIN FOR USE IN HIGH-THROUGHPUT ANTIVIRAL ASSAYS

(75) Inventors: Jason Peter Roth, Watkinsville, GA (US); Dale Barnard, Nibley, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/814,031

(22) Filed: Jun. 11, 2010

Related U.S. Application Data
(60) Provisional application No. 61/186,239, filed on Jun. 11, 2009.

(51) Int. Cl. C12Q 1/70; C12N 7/01; C07H 21/04; C12P 19/34; C12Q 1/68; C12N 7/02

(52) U.S. Cl. 435/5; 435/235.1; 536/23.72; 435/91.2; 435/6

(57) ABSTRACT

Disclosed herein is a recombinant human parainfluenza virus expressing the enhanced green fluorescent protein. Methods of making and methods of using a recombinant human parainfluenza virus expressing the enhanced green fluorescent protein are also disclosed. A recombinant human parainfluenza virus expressing the enhanced green fluorescent protein was rescued and evaluated for its use in antiviral assays. Without limiting the invention, in one example, there is provided a cDNA clone of SEQ ID NO: 1.

### CROSS-REFERENCE TO RELATED APPLICATIONS

This application claims the priority of U.S. Provisional Application Ser. No. 61/186,239, entitled “Human parainfluenza virus type 3 expressing the enhanced green fluorescent protein for use in high-throughput antiviral assays,” filed on 11 Jun. 2009, the entire contents and substance of which are hereby incorporated by reference herein.

### STATEMENT REGARDING FEDERALLY SPONSORED RESEARCH OR DEVELOPMENT

This invention was made with government support under contract NØ1-A1-30048 awarded by the National Institutes of Health. The government has certain rights in the invention.

### REFERENCE TO SEQUENCE LISTING, A TABLE, OR A COMPUTER PROGRAM LISTING COMPACT DISK APPENDIX

This application includes a 147 KB computer readable sequence listing created on Jun. 11, 2010 using Pat-In 3.5 and entitled “HPIV3_ST25_submit,” the entire contents of which is hereby incorporated herein.

### BACKGROUND OF THE INVENTION

Human parainfluenza type 3 (HPIV-3) is classified in the Paramyxovirinae subfamily, which comprises nonsegmented, negative-sense, single-stranded RNA viruses. The HPIV-3 genome consists of six transcriptional gene units composed of one or more genes whose proteins are, in order, nucleocapsid protein, phosphoprotein, matrix protein, fusion protein, hemagglutinin-neuraminidase protein, and large protein. Some members of the Paramyxovirinae subfamily also express accessory proteins from the phosphoprotein gene, for example, but not limited to, C, Y, W, V, and D proteins. The transcriptional units are flanked by 3′ leader and 5′ trailer untranslated regions that are essential for viral transcription and replication regulation. Each transcriptional unit is separated by gene end, intercistronic, and gene start sequences.

During viral mRNA synthesis, the viral RNA polymerase recognizes the gene end sequence and stutters, adding non-templated adenosine residues to create a poly-A tail. The viral RNA polymerase then re-engages viral mRNA transcription at the gene start sequence for the next gene unit. The viral RNA polymerase will sometimes fail to re-engage mRNA transcription for the downstream gene, which results in fewer mRNA transcripts for downstream genes compared to upstream genes transcribed from the same template. This phenomenon is termed transcriptional polarity and ultimately leads to less protein expression from downstream gene units. The decreased protein expression may occur in a decreasing gradient of protein expression. Transcriptional polarity can be used advantageously for regulation purposes by cloning foreign genes into various locations on the viral genome to substantially control the rate of expression of the foreign gene. The gene for the green fluorescent protein has been cloned directly into the genomes of the ebolavirus and cytomegalovirus.

Several members of the Paramyxovirinae subfamily have been successfully rescued with the use of cDNA clones, including measles virus (MeV), Sendai virus (SeV), and two HPIV-3 viruses (strains 47885 and JS). The cDNA clone methodology has been used to effectively express foreign genes from the genomes of some infectious, recombinant RNA viruses. The Ebola virus glycoprotein and the respiratory syncytial virus (RSV) fusion protein have been expressed from recombinant HPIV-3 and SeV viruses, respectively, and have resulted in protective immunity against Ebola virus and RSV, respectively.

Another area where the insertion of a foreign gene into a recombinant virus has been beneficial is for the expression of a reporter gene for the purpose of tracing the viral infection. A recombinant HPIV-3, strain JS, was engineered to express the EGFP protein and was used to trace the infection of HPIV-3 exclusively to the apical surface of ciliated airway epithelium by attaching to α2-6-linked sialic acid receptors. Recombinant virus expressing reporter genes may be used to detect and measure virus replication in real-time.

### BRIEF SUMMARY OF THE INVENTION

### Definitions

“rHPIV3-EGFP,” as used herein, means a recombinant human parainfluenza type 3 virus capable of expressing enhanced green fluorescent protein.
“EGFP,” as used herein, means an enhanced green fluorescent protein.
“HPIV-3 WT,” as used herein, means a wild type human parainfluenza type 3 virus.
“HPIV-3,” as used herein, means a human parainfluenza type 3 virus.
“rHIPV3,” as used herein, means a recombinant human parainfluenza type 3 virus. It may be used as a control to rHPIV3-EGFP, and/or as a precursor in cloning or rescuing a rHPIV3-EGFP.
“NP,” as used herein, means a human parainfluenza type 3 Nucleocapsid protein.
“P,” as used herein, means a human parainfluenza type 3 Phosphoprotein.
“L,” as used herein, means a human parainfluenza type 3 Large protein.
“CPE,” as used herein, means cytopathic effect.
“MOI,” as used herein, means multiplicity of infection.
“ORF,” as used herein, means open reading frame.
“Encodes,” as used herein, means to specify, after decoding by transcription and translation, the sequence of amino acids in a protein.
“Expresses,” as used herein, means to manifest or be capable of manifesting the effects of a gene or genetic trait.
“Providing,” as used herein, means to give something useful or necessary.
“Assembling,” as used herein, means to create by putting components or members together.
“Purifying,” as used herein, means to make substantially free of impurities.
“Infecting,” as used herein, means to contaminate with a disease or microorganism or an agent or a gene derived from a disease or microorganism, or a recombinant form thereof.
“Optionally,” as used herein, means possible but not necessary.

In broad embodiment, the present invention relates to a recombinant HPIV-3 virus (rHPIV3-EGFP) that encodes and expresses the enhanced green fluorescent protein (EGFP), methods of making rHPIV3-EGFP, and methods of using rHPIV3-EGFP in antiviral assays. An rHPIV3-EGFP was rescued and evaluated for its use in antiviral assays by comparing it side-by-side with both HPIV-3 wild-type (HPIV-3 WT) and recombinant HPIV-3 strains that do not express enhanced green fluorescent protein. Without limiting the invention, in one example, only slight differences in virulence between the rHPIV3-EGFP virus and the HPIV-3 WT virus in cell culture were observed. The observed slight differences in virulence between the rHPIV3-EGFP virus and the HPIV-3 WT virus in cell culture validate the substituting of an rHPIV3-EGFP for the HPIV-3 WT virus in primary, high-throughput antiviral assays.

In one embodiment, there is provided a modified cDNA clone of the positive sense antigenome of an rHPIV3-EGFP at least 95%, at least 98%, or at least %100 identical to the nucleotide sequence of SEQ ID NO: 1. In a related embodiment, there is provided a cDNA clone of the positive sense antigenome of a human parainfluenza type 3 virus at least 95%, at least 98%, or at least %100 identical to the nucleotide sequence of SEQ ID NO: 2, into which an EGFP encoding nucleotide sequence has been cloned in a position corresponding to a first, second, third, fourth, fifth, sixth or seventh transcriptional unit. Optionally, one or more viral proteins at least 95% identical, or at least 98% identical, or at least 100% identical, to one or more proteins selected from a group consisting of Nucleocapsid protein (SEQ ID NO: 3), Phosphoprotein (SEQ ID NO: 4), C protein (SEQ ID NO: 5), Matrix protein (SEQ ID NO: 6), Fusion Protein (SEQ ID NO: 7), HN protein (SEQ ID NO: 8), and Large protein (SEQ ID NO: 9) are used to enhance viral rescue or an antiviral assay. Also provided are methods to rescue an infectious, recombinant RNA virus from a cDNA clone, and for measuring viral replication from a viral expressed reporter gene. Without limiting the invention, in one example, the cDNA clone is a DNA clone of an HPIV-3 antigenome and is used to rescue an infectious rHPIV3-EGFP.

Also disclosed are methods for the insertion of an enhanced green fluorescent protein (EGFP) gene into a human parainfluenza virus type 3 (HPIV-3) antigenome and rescue of a recombinant, infectious virus. Without limiting the invention, in one embodiment, the first step in the process includes generating a cDNA clone copied from viral RNA isolated from an HPIV-3 wildtype infection. In a second step the EGFP gene is inserted into the viral antigenome. Optionally, said insertion of EGFP gene into the viral antigenome results in independent expression during virus replication. In a third step the viral support genes that are responsible for viral replication are cloned into an expression plasmid. Optionally, the expression plasmid into which viral support genes are cloned may be a T7 expression plasmid. Alternatively, other plasmids common in the art may be used. In a fourth step, an infectious, rHPIV3-EGFP virus is rescued from the cDNA clone. The rescue of the rHPIV3-EGFP virus may occur with the assistance of viral support genes and viral proteins expressed therefrom. Optionally, the viral support genes may be cloned support genes. Optionally the cloned support genes may be enhanced or altered to increase rescue of the rHPIV3-EGFP virus. Optionally, the viral support genes and proteins expressed therefrom may be provided by way of a vector or vectors separate from the vector providing the rHPIV3-EGFP virus.

Cells infected with rHPIV3-EGFP virus may emit green fluorescence. Optionally, said fluorescence can be photographed and quantitated. Without limiting the invention, said fluorescence emitted from cells infected with rHPIV3-EGFP may be detected and, optionally, quantitated for use, for example, as an infection tracer or as a direct measure of virus replication.

The generation of rHPIV3-EGFP, an infectious, recombinant human parainfluenza virus type 3 (rHPIV-3) that expresses the enhanced green fluorescent protein (EGFP) is herein disclosed. Optionally, the green fluorescence emitted from cells infected with rHPIV3-EGFP can be detected and quantitated for use as an infection tracer or as a direct measure of virus replication. To study the effects of gene mutation or foreign gene expression of an RNA virus, infectious, recombinant virus may be rescued from a viral cDNA clone of a negative-sense RNA virus. Optionally, the rescuing of a negative-sense RNA virus relies on the generation of a full-length viral antigenomic RNA from the viral cDNA clone. The viral nucleocapsid protein (NP), phosphoprotein (P), and large protein (L) proteins only recognize and interact with viral RNA; therefore, it is desirable to convert the viral cDNA clone to viral antigenomic RNA of proper length and composition. Without limiting the invention, in one embodiment, the transcription of the viral antigenomic RNA is driven by a T7 promoter. Other promoters useful in the art may be chosen for transcription of the viral antigenomic RNA. Optionally, the chosen promoter, for example the T7 promoter, is strategically placed immediately upstream of the first nucleotide of the 5′ end of the viral antigenome. Optionally, the rescue of infectious, recombinant virus is enhanced when the T7 promoter and the first nucleotide of the viral antigenome are separated by two guanosine residues. The forward primer used in amplifying a 5.3-kb antigenomic cDNA segment may include the T7 promoter adjacent to the 5′ end of the HPIV-3 antigenome separated by two guanosines. Optionally, on the 3′ end of the antigenome, an antigenomic hepatitis delta virus ribozyme may be positioned immediately following the last nucleotide of the viral antigenome. Optionally, the ribozyme may self-cleave from the viral RNA antigenome leaving the full-length virus RNA antigenome intact and at a proper length. The Rib polylinker may encode the hepatitis delta ribozyme adjacent to the 3′ end of the antigenome.

In some embodiments, for enhancing expression of the EGFP gene from the viral antigenome, the EGFP gene may be altered to mimic a viral gene by the addition of a nucleotide sequence encoding viral mRNA regulation sequences. Viral mRNA regulation sequences may include sequences native to the virus antigenome into which the EGFP gene is to be inserted. Alternatively, viral mRNA sequences may come from other strains or even completely different viruses.

The HPIV-3 antigenome consists of six distinct transcriptional units, each of which encode for one or more genes. Without limiting the invention, in one embodiment, the EGFP gene is inserted as a seventh transcriptional unit. Each transcriptional unit is separated by a gene end, intercistronic, and gene start sequences. Therefore, when inserting the EGFP gene as a seventh transcriptional unit, the insertion may contain the gene end, intercistronic, and gene start sequences to be effectively expressed through viral mRNA transcription. The reverse primer used to amplify the EGFP gene may comprise regulation sequences. The regulation sequences are optionally located between the EGFP gene and the HPIV-3 nucleocapsid gene. Alternatively, the EGFP gene may be inserted as the first, second, third, fourth, fifth, or sixth transcriptional unit. Without limiting the invention, the selection of the first, second, third, fourth, fifth, sixth, or seventh transcriptional unit position for insertion of EGFP may be chosen based on the desired expression level of EGFP.

In some embodiments, during both virus rescue and normal infection, virus replication may be most efficient when the length of the complete viral genome is a factor of six. Without limiting the invention, primers designed to amplify the EGFP gene may result in an insertion of nucleotides comprising a factor of six. Again, without limiting the invention, in one example, the primers designed to amplify the EGFP gene and subsequent digestion may result in an insertion of 852 nucleotides, which is a factor of six. Optionally, there is a bipartite replication promoter, which may consist of three equally-spaced guanosine residues at viral antigenome locations 79, 85, and 91, and may coincide with the EGFP gene transcription unit insertion site. This location represents one turn of the nucleocapsid helical structure and may co-regulate viral replication through the assembly and binding of the L-P complex with the encapsidated RNA genome. Optionally, the addition of the promoter sequence to the forward primer used to amplify the EGFP gene may restore the bipartite replication promoter and enhance the rescue of rHPIV3-EGFP.

Without limiting the invention, in some embodiments, the present invention helps overcome problems with rescuing negative-sense RNA viruses. The ability to rescue negative-sense RNA viruses can be problematic because the viruses commonly replicate in the cytoplasm and, thus, may not have access to the host cell's transcriptional machinery in the nucleus. In addition, genomic viral RNA of negative-sense RNA viruses, which has negative polarity, lacks the signals necessary to initiate eukaryotic protein translation while in the cytoplasm. Therefore, during normal infection and viral replication, the viral proteins necessary for viral RNA synthesis are commonly packaged into virions in active transcriptase-replicase complexes for immediate replication upon infection. Without limiting the invention, in some embodiments of the rescue of a recombinant negative-sense virus, all components required for viral replication are provided in some form, including the full-length viral antigenomic RNA and viral proteins NP, P, and L.

In one embodiment, to express the NP, P, and L proteins, the genes encoding said proteins are cloned into a T7 expression vector, which may be transcribed by T7 RNA polymerase and translated by the ribosomes of the host cell. Alternatively, other vectors and polymerases known in the art may be used in place of the T7 expression vector and T7 RNA polymerase. Without limiting the invention, the T7 RNA polymerase, used to transcribe the viral antigenomic RNA and NP, P, and L transcripts, may be supplied from a recombinant vaccinia virus, vTF7-3, which may be used to infect the host cell during the rescue procedure. Optionally, to select for the rescued rHPIV3-EGFP virus and inhibit the replication of vTF7-3, which may contaminate the infectious, recombinant virus, an antiviral compound may be added to the medium for protection of the infected cells. Without limiting the invention the antiviral compound may be cytosine β-D-arabinofuranoside (Ara-C). Alternatively, other antiviral compounds known in the art may be used.

### BRIEF DESCRIPTION OF THE DRAWINGS

FIG. 1 is a depiction of an embodiment of rHPIV3-EGFP. The EGFP is inserted into the rHPIV3 viral antigenome. As pictured, the EGFP is inserted as the first gene or transcriptional unit.

FIG. 2 shows mutations destroying a natural SphI site (* indicating destroyed) for the construction of rHPIV3 and rHPIV3-EGFP.

FIG. 3 shows electrophoresis of PCR fragments from HPIV-3 WT (lane 1), rHPIV3 (lane 2), and rHPIV3-EGFP (lane 3) digested with SphI.

FIG. 4 shows growth curves for HPIV-3 WT (▪), rHIPV3 (▴) and rHPIV-EGFP ().

FIG. 5 shows the CPE produced by rHPIV3-EGFP () and HPIV-3 WT (▪) viruses in infected MA-104 cells that were monitored for 7 days. CPE was measured by NR uptake.

FIG. 6 shows the relative expression of L gene transcription as a function of hours post infection for rHPIV3-EGFP () and HPIV-3 WT (▪).

FIG. 7 shows the relative genomic expression as a function of days post infection for HPIV-3 WT (▪) and rHPIV-EGFP ().

FIG. 8 show an EGFP expression curve for 96-well plates seeded with MA-104 cells and infected with rHPIV3-EGFP at differing MOIs: 1 (♦), 0.1 (▪), 0.01 (▴), 0.001 (▪).

### DETAILED DESCRIPTION OF THE INVENTION

Without limiting the scope of the invention as demonstrated and envisioned by the accompanying examples and embodiments, disclosed herein are the modified cDNA clone of SEQ ID NO: 1 for the antigenome of a human parainfluenza virus type 3 antigenome, a cDNA clone of SEQ ID. NO: 2 for the unmodified antigenome of the human parainfluenza virus type 3 antigenome, and cDNA clones for overlapping complementary DNA (cDNA) strands, encompassing viral bases 1-5267, 5249-11366, and 11284-15453, which were generated from RNA isolated from a HPIV-3 WT, strain 14702 (SEQ ID NO: 2), infection. The disclosed clones are useful in recovering a recombinant infectious parainfluenza virus and in a high throughput antiviral screen, which are also disclosed herein.

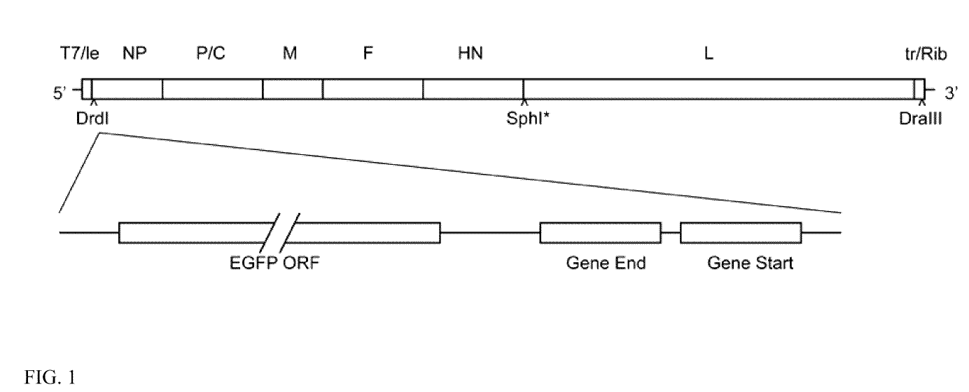

Referring now to FIG. 1, there is shown a depiction of an embodiment of rHPIV3-EGFP. The EGFP gene is inserted into a recombinant human parainfluenza type 3 viral antigenome. As pictured, the EGFP gene is inserted as the first gene or transcriptional unit. Alternatively, the EGFP gene may be inserted as the second, third, fourth, fifth, sixth, or seventh transcriptional unit. The 852 by EGFP PCR product was inserted into a natural DrdI site located between the N gene's start signal and start codon. The reverse primer used to amplify EGFP's ORF was designed to encode the HPIV-3 gene end and gene start signals. T7/le indicates that a T7 promoter precedes the rHPIV3 5′ antigenomic leader sequence. tr/Rib indicates a hepatitis delta ribozyme immediately follows the rHPIV3 3′ antigenomic trailer sequence.

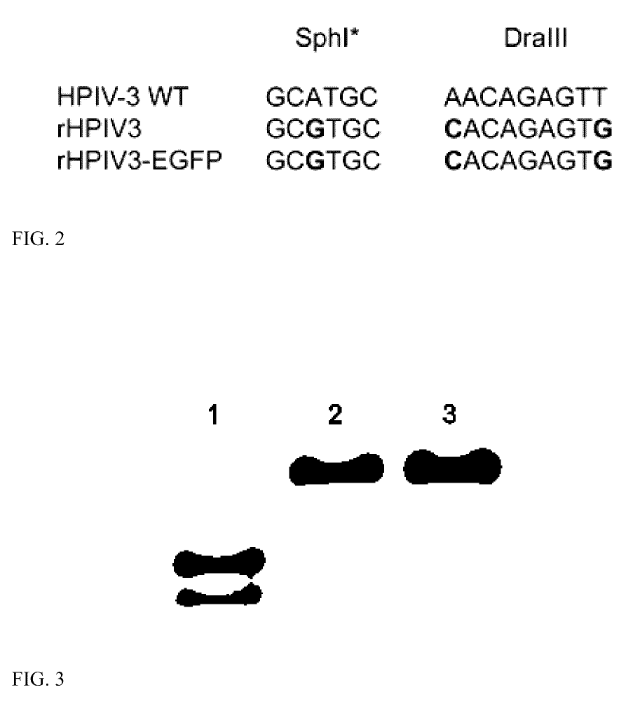

Referring now to FIG. 2, there are shown three intentional mutations made to rHPIV3 and rHPIV3-EGFP, and recombinant markers: A to G, destroying a natural SphI site (* indicating destroyed) located in the 5′ noncoding region of the L gene (as pictured in FIG. 1), and A to C and T to G, creation of a unique DraIII site located within the 3′ trailer region (as pictured in FIG. 1).

Referring now to FIG. 3, there is shown a depiction of an electrophoresis of PCR fragments from (1) HPIV-3 WT, (2) rHPIV3, and (3) rHPIV3-EGFP, digested with SphI.

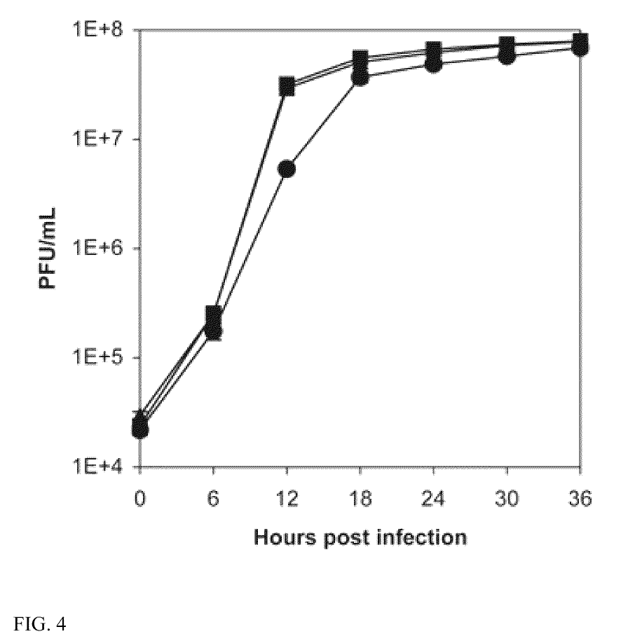

Referring now to FIGS. 4 through 7, there are shown infectious assays comparing HPIV-3 WT, rHPIV3, rHPIV3-EGFP viruses.

Referring now to FIG. 4, there is shown a single step growth curve. HPIV-3 WT (▪), rHPIV3 (▴), and rHPIV3-EGFP () were used to separately infect 12-well plates seeded with MA-104 cells at MOI=2. Individual cells were harvested every 6 h, including 0 h, and viral titers were measured by plaque assay. The growth curve for rHPIV3 was not significantly different compared to the growth curve for HPIV-3 WT (p>0.01), while the growth curve for rHPIV3-EGFP was significantly different compared to the growth curves for HPIV-3 WT and rHPIV3 (p<0.01).

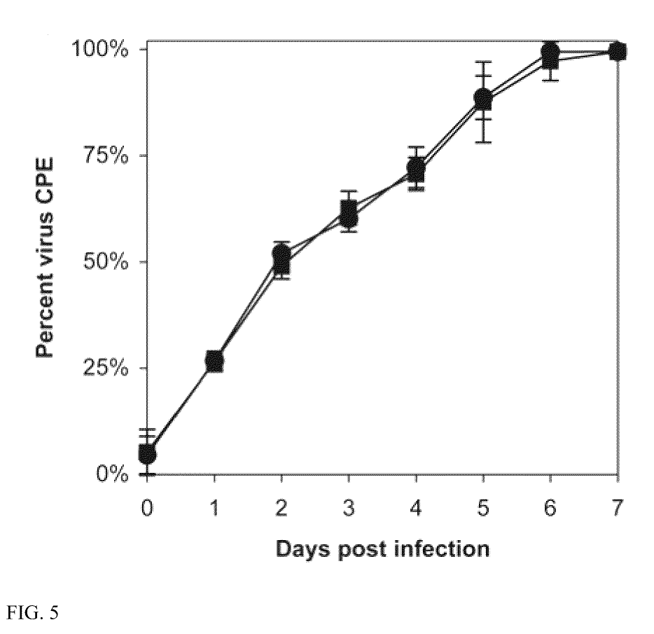

Referring now to FIG. 5, there is shown a time course of virus induced CPE. HPIV-3 WT (▪) and rHPIV3-EGFP () were used to infect MA-104 cells at MOI=0.1 in 96-well plates. Each day, including day 0, the cells of one plate were stained with NR for 2 h, washed once with PBS, and the NR extracted with ethanol:Sörenson's citrate buffer for 30 min rocking Absorbance was measured on a spectrophotometer using 540 and 405 nm wavelengths. Percents were calculated based on NR reduction in infected cells compared to uninfected cell controls. No significant differences were detected (p>0.01).

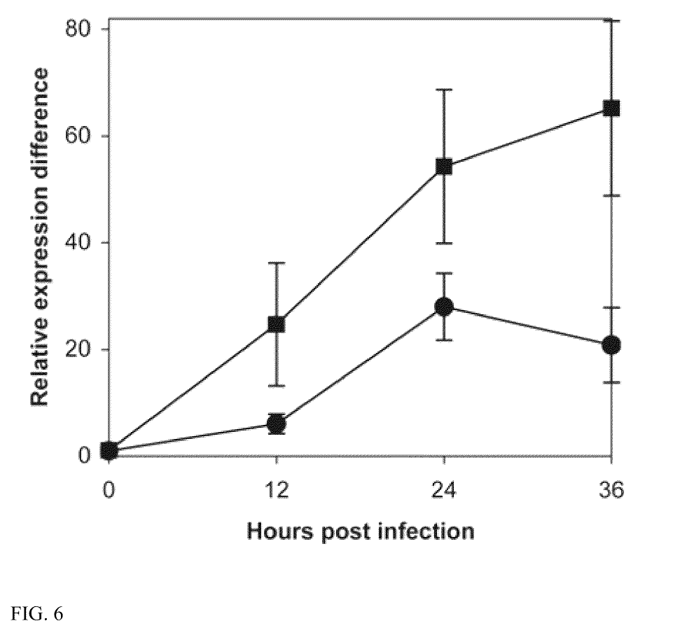

Referring now to FIG. 6, relative expression differences in L gene transcription were measured by QRT-PCR. MA-104 cells infected with HPIV-3 WT (▪) and rHPIV3-EGFP () were harvested at specified time points. The reverse transcriptase reaction was primed with an Oligo(dT)20 primer for L gene transcription. The cDNAs were amplified using HPIV-3 specific primers, which were tagged with FAM. Delta-C_{T} relative expression differences were calculated for each virus at each time point, using the 0 h measurement for each virus as the calibrator. Significant reductions were seen in L gene transcription compared to HPIV-3 WT (p<0.01). All Y-axis values on all graphs represent the mean±S.D. of duplicate assays.

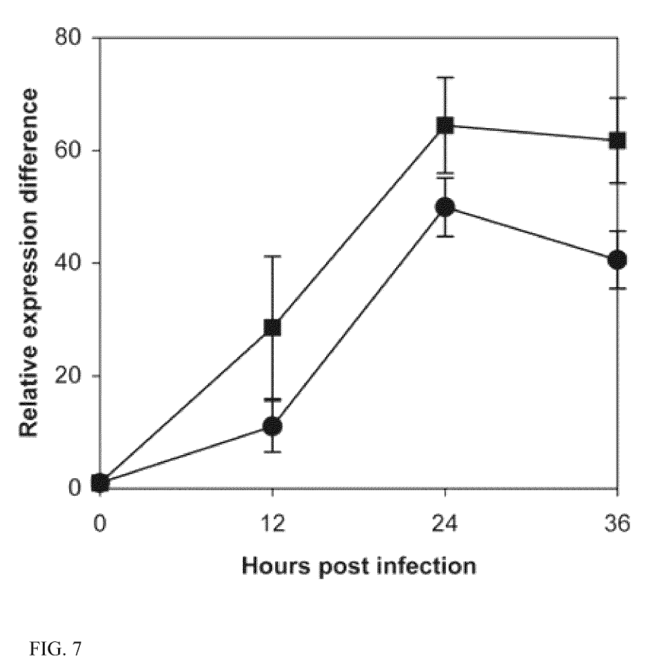

Referring now to FIG. 7, relative expression differences in genomic replication were measured by QRT-PCR. MA-104 cells infected with HPIV-3 WT (▪) and rHPIV3-EGFP () were harvested at specified time points. The reverse transcriptase reaction was primed with an HPIV-3 specific primer 5′-AATTATAAAAAACTTAGGAGTAAAG-3′ (SEQ ID NO: 10), which straddles the intergenic region between the fusion and hemagglutinin-neuraminidase genes and anneals to viral, negative-sense, genomic RNA. The cDNAs were amplified using HPIV-3 specific primers, which were tagged with FAM. Delta-C_{T} relative expression differences were calculated for each virus at each time point, using the 0 h measurement for each virus as the calibrator. Significant reductions were seen in rHPIV3-EGFP genomic replication and L gene transcription compared to HPIV-3 WT (p<0.01). All Y-axis values on all graphs represent the mean±S.D. of duplicate assays.

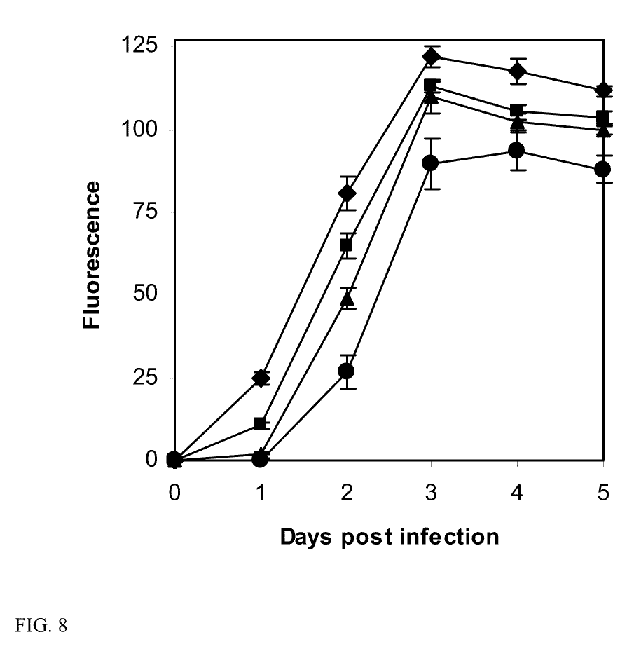

Referring now to FIG. 8, there is shown an EGFP expression curve. 96-Well plates were seeded with MA-104 cells and infected with rHPIV3-EGFP at differing MOIs: 1 (♦), 0.1 (▪), 0.01 (▴), 0.001 (). Each day, including day 0, the cell monolayer was washed once with PBS and fluorescence measured. HPIV-3 WT and rHPIV3 infections were also done in parallel but no fluorescence was detected. All Y-axis values represent the mean±S.D. of duplicate assays.

The following descriptions, methods, and disclosures may be useful in practicing various embodiments of the invention.

### Materials and Methods

### Cells and Viruses

Human cervical carcinoma cells (HeLa) were obtained from American Type Culture Collection (ATCC, Manassas, Va.) and maintained at 37° C. and 5% CO₂ in minimal essential medium (MEM, Hyclone Laboratories, Logan, Utah) supplemented with 10% fetal bovine serum (FBS, Hyclone Laboratories), 0.1 mM non-essential amino acids (NEAA, Invitrogen, Carlsbad, Calif.), and 1 mM sodium pyruvate (Invitrogen). Embryonic African green monkey kidney cells (MA-104) were obtained from ATCC and maintained at 37° C. and 5% CO₂ in MEM supplemented with 10% FBS. A recombinant vaccinia virus that expresses the bacteriophage T7 RNA polymerase, vTF7-3, generously provided by Dr. Bernard Moss, was propagated in HeLa cells. HPIV-3 WT, isolate 14702, (J. Bouvin, Hosp. St. Justine, Montreal, Canada) was propagated in MA-104 cells. During the antiviral assays, MA-104 cells were incubated in MEM supplemented with 2% FBS and 50 μg/ml gentamicin (Sigma Chemical Company, St. Louis, Mo.).

### Antiviral Compounds

2-[(2R,3R,4S,5R)-3,4-Dihydroxy-5-(hydroxymethyl)oxolan-2-yl]-3-sulfanylidene-1,2,4-triazin-5-one (2-thio-6-azauridine) was obtained from Sigma and the remainder of the antiviral compounds, including 1-[(2R,3R,4S,5R)-3,4-dihydroxy-5-(hydroxymethyl)oxolan-2-yl]-1,2,4-triazole-3-carboxamide (ribavirin), 1-[(2R,3R,4S,5R)-3,4-dihydroxy-5-(hydroxymethyl)oxolan-2-yl]-1,2,4-triazole-3-carboximidamide (ribamidine), 2-[(2R,3R,4S,5R)-3,4-dihydroxy-5-(hydroxymethyl)oxolan-2-yl]-1,3-selenazole-4-carboxamide (selenazofurin), 1-[(2R,3R,4S,5R)-3,4-dihydroxy-5-(hydroxymethyl)oxolan-2-yl]-5-ethynylimidazole-4-carboxamide (EICAR), 6-amino-1-[(2R,3R,4S,5R)-3,4-dihydroxy-5-(hydroxymethyl)oxolan-2-yl]-5H-imidazo[4,5-c]pyridin-4-one (3-deazaguanosine), and 1-(4-methoxybenzyloxy) adenosine, were obtained from the repository of the NIAID Antiviral Substances Program, NIH. Also obtained from the repository were CS-978, CS-1164, CS-1196, CS-1227, PSI-0194, PSI-5067, PSI-5095, PSI-5098, PSI-5452, PSI-5449, PSI-5741, PSI-5746, PSI-5747, PSI-5852, and PSI-5990 nucleoside analog compounds that were submitted by and used with permission from Dr. Michael Otto of Pharmasset, Inc.

### Plasmid Construction

Three overlapping complementary DNA (cDNA) strands, encompassing viral bases 1-5267, 5249-11366, and 11284-15453, were generated from RNA isolated from a HPIV-3 WT, strain 14702, infection. Forward and reverse primers were derived from the consensus sequence between three known HPIV-3 strains, 47885, JS, GPv. To generate the 1-5267 cDNA segment the 5′CCGACGTCTTAATTAATACGACTCACTATAGGACCAAACAAGAGAAGAAACTT-3′forward primer (SEQ ID NO: 11), which contains AatII and PacI restrictions sites (bolded) and a T7 promoter (underlined) and the 5′-GGTCACCACAAGAGTTAGA-3′ (SEQ ID NO: 12) reverse primer were used. To generate the 5249-11366 cDNA segment the 5′-TCTAACTCTTGTGGTGACC-3′ (SEQ ID NO: 13) forward primer, which contains a natural BstEII restriction site (bolded) and the 5′-ATTCATCCCAAGGGCAATA-3′ (SEQ ID NO: 14) reverse primer were used. To generate the 11284-15453 cDNA segment the 5′-AGAATGGTTATTCACCTGTTC-3′ (SEQ ID NO: 15) forward primer and the 5′-GAGAAGCACTCTGTGTGGTAT-3′ (SEQ ID NO: 16) reverse primer, which contains a mutated DraIII restriction site (bolded) with the two mutations, A to C and T to G (underlined), were used. The cDNA segments were inserted into the SmaI site of pUC19 (New England Biolabs, NEB, Ipswich, Mass.). Clones were sequenced in both directions to assure accuracy. An SphI site in the 5249-11366 cDNA segment was destroyed by mutating A to G, viral base position 8635, using the QuikChange® XL Site-Directed Mutagenesis (Stratagene, La Jolla, Calif.) kit and the forward primer, 5′-pCTTAGGAGCAAAGCGTGCTCAGAAAATGGACACTG-3′ (SEQ ID NO: 17), and reverse primer, 5′-pCAGTGTCCATTTTCTGAGCACGCTTTGCTCCTAAG-3′ (SEQ ID NO: 18). For SEQ ID NO: 17 and 18, “p” represents phosphorylation of the primer that aids in cloning.

To confirm the sequence of the 3′ end of the HPIV-3 WT genome, a poly(A) tail was added to the 3′ end of the isolated HPIV-3 WT RNA using the Poly(A) Tailing Kit (Ambion, Austin, Tex.). The tailed RNA was amplified by RT-PCR using a 60 nucleotide (nt) poly(T) oligonucleotide (SEQ ID NO: 19), as the forward primer, and 5′-TCGTTTTAGATCCTTCTCAATCA-3′ (SEQ ID NO: 20), as the reverse primer. To sequence the 5′ end of the HPIV-3 WT genome, the SMART™ RACE cDNA Amplification Kit (Clontech) was used. An HPIV-3 WT specific primer, 5′-GGAAGGAGCCATCGGCAAATCAGAAG-3′ (SEQ ID NO: 21), was used to prime cDNA synthesis and also used in the PCR amplification step as the forward primer. The PCR products from both the 3′ and 5′ reactions were sequenced to complete the HPIV-3 WT 14702 genome (GenBank accession no. EU424062).

Two oligonucleotides were generated to contain a 14 base pair overlap between each other, 5′-TTTTTGTGCGCCCAATACGCAAACCGCCTCTCCCCGCGCGTTGGCCGTTAATTAAGAGG GTGACCCTGCACAGAGTGCC-3′ (SEQ ID NO: 22) and 5′-TTTTTGTAAAAAACCCCTCAAGACCCGTTTAGAGGCCCCAAGGGGTTATGCTAGTTAGG TACCCGGGCACTCTGTGCAG-3′ (SEQ ID NO: 23). The oligonucleotides were annealed together and extended using Sequenase™ Version 2.0 DNA polymerase (USB Corporation, Cleveland, Ohio). The fragment was inserted into the SmaI site of pUC19 and named pUC19-A. The completed segment contained the PacI, BstEII, DraIII, SmaI, and KpnI sites (bolded), a T7 termination sequence (underlined), and two vaccinia virus termination sequences flanking each end (italicized). A second set of oligonucleotides were annealed, extended, and inserted into pUC19 in the same manner, 5′-ACCACACAGAGTGCTTCTCTTGTTTGGTGGGTCGGCATGGCATCTCCACCTCCTCGCGGT CCGACCT-3′ (SEQ ID NO: 24) and 5′-GGCCGGTACCTCCCTTAGCCATCCGAGTGGACGACGTCCTCCTTCGGATGCCCAGGTC GGACCGCGA-3′ (SEQ ID NO: 26). The completed segment, pUC19-R, contained the KpnI and DraIII restriction sites (bolded), the antigenomic hepatitis delta virus ribozyme (underlined) (Perrotta and Been, 1991), and the viral bases 15435-15462 (italicized). pUC19-R, digested with DraIII and KpnI, produced a 108 base pair (bp) segment that was inserted into the same sites of pUC19-A and was renamed pUC19-B. After destroying the native SphI site in pUC19-B and renamed pUC19-C, an adapter, using the oligonucleotides 5′-GTGACCGCGCATGCCCACAGA-3′ (SEQ ID NO: 27) and 5′-GTGGGCATGCGCG-3′ (SEQ ID NO: 28), was inserted into the DraIII and BstEII sites of pUC19-C to encode a SphI site (bolded) and renamed pUC19-D. Next, the 5249-11366 cDNA segment was digested with BstEII and SphI, inserted into the same sites of pUC19-D, and renamed pUC19-F. Then, the 1-5267 cDNA segment was digested with PacI and BstEII, inserted into the same sites of pUC19-F, and renamed pUC19-G. Finally, the 11284-15453 cDNA segment was digested with DraIII and SphI, inserted into the same sites of pUC19-G, and renamed pUC19-H.

The 1-5267 cDNA segment, digested with AatII and BstEII, was inserted into the same sites of pACYC177 (NEB) and named p177-1Gen. The open reading frame of EGFP was amplified by PCR using pEGFP (Clontech, Mountain View, Calif.) as the template and the forward, 5′-TTGACTAGAAGGTCAAGAACCTGCAGGTCGACTCTAGAGGAT-3′ (SEQ ID NO: 29), and reverse, 5′-TTGACCTTCTAGTCAATGTCTTTAATCCTAAGTTTTTCTTATTTATTAACCGGCGCTCA GTTGGAAT-3′ (SEQ ID NO: 30), primers. Both primers contain a DrdI restriction site (bolded) on their 5′ ends. The reverse primer also includes the HPIV-3 WT gene end, intercistronic, and gene start signals (underlined). The 868 by band was purified and inserted into the SmaI site of pUC19, which was renamed pUC19-EGFP. pUC19-EGFP, digested with DrdI, produced an 852 by band that was inserted into the same site of p177-1Gen and named p177-1Gen-E. A 1-6119 antigenomic cDNA segment, containing EGFP, was digested out of p177-1Gen-E with PacI and BstEII, inserted into the same sites of pUC19-F, and named pUC19-I. Finally, the 11284-15453 cDNA segment was digested with DraIII and SphI, inserted into the same sites of pUC19-I, and renamed pUC19-J.

Three PCR products encompassing the nucleocapsid protein (NP), phosphoprotein (P), and large protein (L) were inserted into the SmaI site of pUC19 and named pUC19-NP, pUC19-P, and pUC19-L. The following three sets of forward and reverse primers were used: NP, 5′-GAAGGTCAAGAAAAGGGAACTCT-3′ (SEQ ID NO: 31) and 5′-TTGATTCGATTAGTTGCTTCCA-3′ (SEQ ID NO: 32); P, 5′-TGATGGAAAGCGACGCTAAA-3′(SEQ ID NO: 33) and 5′-GGATCATTGGCAATTGTTGA-3′ (SEQ ID NO: 34); L, 5′-GCGTGCTCAGAAAATGGACA-3′ (SEQ ID NO: 35) and 5′-CCTTAGGCTTAAAGATAAAGGTTAGGA-3′ (SEQ ID NO: 36). The start codon (bolded) for the HPIV-3 WT accessory C protein, located on the P forward primer, was mutated from T to C (underlined) to silence its expression. pUC19-NP, pUC19-P, and pUC19-L were digested with SalI and KpnI and the 1.5, 2, 7 kb bands, respectively, were inserted into the same sites of pTNT™ (Promega) and named pTNT-NP, pTNT-P, and pTNT-L.

Rescue of Infectious Virus from cDNA

HeLa cells, seeded in a 12-well plate, were infected with 5.4×10⁵ PFU of vTF7-3 at 1 multiplicity of infection (MOI) for 1 hour. The virus and medium were removed and replaced with Opti-MEM® (Invitrogen), containing 0.1 mM NEAA. The three support plasmids, 0.8 μg of pTNT-NP, 1.6 μg of pTNT-P, and 0.04 μg of pTNT-L, were cotransfected along with 0.4 μg of either pUC19-H or pUC19-J, using Lipofectamine™ 2000 (Invitrogen) for 4-5 hours at 37° C. MEM supplemented with 20% FBS, 0.1 mM NEAA, 1 mM sodium pyruvate, and 250 μg/mL of Cytosine β-D-arabinofuranoside (Ara-C, Sigma) was added to each transfection and incubated for 48 hours at 37° C. The transfected cells were scraped and the supernatants were frozen at −80° C. The rescued rHPIV3, pUC19-H transfection, and rHPIV3-EGFP, pUC19-J transfection, viruses were amplified on MA-104 cells, supplemented with 250 μg/mL Ara-C, for 3-4 days at 37° C. The cells were scraped and the supernatants were frozen at −80° C. Each virus was purified by picking agarose plugs over isolated plaques on MA-104 cells in the absence of Ara-C. Each plug was placed in MEM and froze at −80° C. The media, containing the plug and isolated virus, was used to infect MA-104 cells to amplify the virus for 3-4 days at 37° C. The purification and amplification steps were repeated two more times. The rHPIV3, rHPIV3-EGFP, and HPIV3 WT viruses were amplified on MA-104 cells for 3 days at 37° C. The infected cells were scraped and the supernatants were frozen at −80° C., which were used for further testing. Sequencing of the 5′ ends of the genomic RNA, isolated from rHPIV3 and rHPIV3-EGFP infections, was repeated using the SMART™ RACE cDNA Amplification Kit to confirm the DraIII genetic markers.

### Viral Infectious Assays

### Plaque Assay

Duplicate dilutions of HPIV-3 WT, rHPIV3, and rHPIV3-EGFP, were used to infect MA-104 cells in quadruplicate. Virus was absorbed for 2 hours, after which, the virus was removed and replaced with an overlay of 1% SeaPlaque® low-melting agarose (ISC BioExpress®, Kaysville, Utah) supplemented with MEM and 0.2% sodium bicarbonate and incubated for 2-3 days at 37° C. Cells were fixed with 3.6% formaldehyde for 2 hours at room temperature, after which, the formaldehyde and agarose overlay was removed and 0.5% crystal violet was added for 5 minutes. After removal of the dye and one rinse with phosphate buffered saline (PBS), the stained plaques were counted. Viral titers were compared and statistically analyzed by unpaired, two-tailed Student's t-test using the Microsoft® Office Excel 2003 software (Redmond, Wash.). In addition, plaques produced by rHPIV3-EGFP were also photographed using an Eclipse TS100 microscope (Nikon, Melville, N.Y.), CoolSNAP digital camera, and RSImage™ software, version 1.7.3, (both from Roper Scientific, Photometrics, Tucson, Ariz.). Fluorescent photographs were taken with the same equipment except under UV light and the B-2A fluorescent filter combination was used, which incorporates excitation wavelengths between 450 and 490 nm and emission filter wavelengths greater than 515 nm.

### One-Step Growth Curve

Duplicate 12-well plates were seeded with MA-104 cells and infected with 1.4×10⁶ PFU of HPIV-3 WT, rHPIV3, and rHPIV3-EGFP viruses, separately, at an MOI=2. After virus was absorbed for 2 hours at 37° C., virus was removed, replaced with fresh MEM supplemented with 2% FBS, and incubated at 37° C. Individual cells were scraped and the supernatants harvested every 6 h starting at the time of virus exposure and frozen at −80° C. At time 0, virus was added but then immediately removed and replaced with fresh medium. Each time point for each virus was plaque titered in quadruplicate following the same method as described above. Each growth curve was compared to the other two curves, individually, and statistically analyzed by analysis of variance (ANOVA) using the Microsoft® Office Excel 2003 software.

### Cytopathic Effect Assay

Ninety-six-well plates were seeded with MA-104 cells and infected with 3.9×10³ PFU of either the HPIV-3 WT or rHPIV3-EGFP virus in duplicate at an MOI=0.1 in quadruplicate wells. The plates were incubated at 37° C. and on each day, including the day of infection, the cells were stained with 0.034% neutral red for 2 hours at 37° C., washed once with PBS, and the NR extracted with ethanol:Sörenson's citrate buffer for 30 min while rocking at room temperature. Absorbance, at 540 and 405 nm wavelengths, was read with an Opsys MR™ spectrophotometer and Revelation Quicklink software, version 4.24 (both from Dynex Technologies, Chantilly, Va.). The two curves was compared and statistically analyzed by ANOVA.

### QRT-PCR Assay

Ninety-six-well plates were seeded with MA-104 cells and infected with 7.8×10⁴ PFU of HPIV-3 WT and rHPIV3-EGFP viruses, separately, in duplicate at an MOI=2. At specific time points; 0, 12, 24, and 36 hours, uninfected and infected cells were harvested using CellsDirect Resuspension and Lysis Buffers (Invitrogen). Each lysate was used as the template for two different reverse transcriptase (RT) reactions. One reaction used a primer specific for the HPIV-3 genome, 5′-AATTATAAAAAACTTAGGAGTAAAG-3′ (SEQ ID NO: 37), and the other reaction used an Oligo(dT)20 primer (Invitrogen). The primers used to PCR amplify the cDNA products from the RT reactions include 5′-CGTTATAGTGCTGCCACAAAGAATAA[FAM]G-3′ (SEQ ID NO: 38) and 5′-ATGGAAGACCAGACGTGCATC-3′ (SEQ ID NO: 39), for genomic replication, and 5′-CGATTAAGGAAAGCGACCTGTAAGTAAT[FAM]G-3′ (SEQ ID NO: 40) and 5′-GAGACACAAATTAGGCGGGAGAT-3′ (SEQ ID NO: 41), for L gene transcription. Platinum® Quantitative PCR SuperMix-UDG (Invitrogen), 200 nM of the forward and reverse LUX™ primers (Invitorgen), and 1/10^{th} of the RT reaction were mixed and added, in triplicate, to Hard-Shell 96-well skirted PCR plates (Bio-Rad Laboratories, Hercules, Calif.). The reaction was run on a DNA Engine Opticon 2 Real-Time PCR Detection System (MJ Research, Waltham, Mass.). The Opticon Monitor™ software, version 3.1.32 (Bio-Rad Laboratories) was used to calculate relative expression differences, Delta-C_{T}, at each time point for each virus, using the Oh for each virus as the calibrator (Pfaffl, 2001). For each assay, the two curves were compared and statistically analyzed by ANOVA.

### Antiviral Sensitivity Assay

An antiviral CPE assay was used to evaluate the antiviral sensitivity profiles of the HPIV-3 WT and rHPIV3-EGFP viruses. Briefly, three compounds: ribavirin (positive control), 2-thio-6-azauridine, and DAS181, were plated in four 10-fold dilutions in five replicates on 96-well plates seeded with MA-104 cells using starting concentrations of 1000, 100, and 1 μg/mL, respectively. Two of five replicates were toxicity controls with no virus added, while the other three replicates were infected with 3.9×10³ PFU of either the HPIV-3 WT or the rHPIV3-EGFP virus at an MOI=0.1. The plates were incubated at 37° C. for 7 days and, after which, the cells of each plate were stained with NR following the same method as described above. The assays were done three times. Fifty percent effective concentrations (EC₅₀) were calculated by linear regression using percents of untreated, uninfected cell and untreated, infected virus controls. EC₅₀ values were compared and statistically analyzed by the unpaired, two-tailed Student's t-test.

### EGFP Expression Assays

Ninety-six-well plates were seeded with MA-104 cells and infected with 3.9×10⁴ PFU of rHPIV3-EGFP in duplicate at an MOI=1. Quadruplicate four 10-fold dilutions of virus were plated and the cultures incubated at 37° C. Each day for 8 days, including the day of infection, the medium was removed and the cell cells were washed with PBS and fresh PBS was added. On the day of infection, the virus was added but then immediately removed and the cells were washed with PBS. EGFP fluorescence was measured with the FMax® fluorometer, using the 485 nm excitation and 538 nm emission filters, and recorded with SOFTmax® PRO software, version 1.3.1, (both from Molecular Devices, Union City, Calif.).

Duplicate 96-well plates were seeded with MA-104 cells. On each plate, 16 wells were infected with 3.9×10³ PFU of rHPIV3-EGFP at an MOI=0.1, while 16 wells were left uninfected as a cell control and 16 wells were left unseeded as a no-cell background control. The plates were incubated for 3 days at 37° C. After incubation, the uninfected and infected cells and unseeded wells were washed with PBS, replaced with fresh PBS, and fluorescence was measured using the FMax® fluorometer. The traditional NR-based assay and Vybrant® MTT Cell Proliferation Assay (Invitrogen) were done following the same 96-well plate format except that cells were treated and results were measured after complete infected cell lysis on day 7. The cells for the NR assay were stained following the same procedure described earlier, while the manufacture's quick disclosure was followed for staining of the cells for the Vybrant® MTT assay. The absorbance values for cells treated with MTT were measured using the 540 nm wavelength, Opsys MR™ spectrophotometer, and Revelation Quicklink software. The CellTiter-Glo® Luminescent Cell Viability Assay (Promega) was also performed using the same 96-well format except that MA-104 cells were seeded on white, half area 96-well plates with a clear bottom. Therefore, plating volumes were reduced by 50% and the CellTiter-Glo® reagent was reduced by 75%, while otherwise following the manufacture's disclosure. Luminescence was measured using the Centro LB 960 luminometer and recorded with MikroWin 2000 software, version 4.34 (both by Berthold Technologies, Oak Ridge, Tenn.).

### EGFP-Based Antiviral Assay

Six 96-well plates were seeded with MA-104 cells to evaluate the NR and EGFP assays in parallel. A format was used allowing seven compounds to be tested per plate. Each compound was plated using four 10-fold dilutions in triplicate and starting the concentration at 1000 μg/mL for ribavirin (positive control), and ribamidine; 100 μg/mL for 2-thio-6-azauridine, 3-deazaguanosine, 1-(4-methoxybenzyloxy) adenosine, selenazofurin, and EICAR; 100 μM for CS-978, CS-1164, CS-1196, CS-1227, PSI-0194, PSI-5067, PSI-5095, PSI-5098, PSI-5452, PSI-5449, PSI-5741, PSI-5746, PSI-5747, PSI-5852, and PSI-5990; and 1 μg/mL for DAS181. Compounds that were reconstituted in DMSO were diluted down to working concentrations of 0.5% DMSO and less to eliminate cell toxicity due to the DMSO. Two of the replicates were infected with 3.9×10³ PFU of rHPIV3-EGFP, at an MOI=0.1, while the remaining replicate served as a toxicity control with no virus added. Three of the plates were incubated at 37° C. for 3 days, after which, the toxicity and cell control cells were stained with NR following the same procedure described earlier. NR fluorescence was measured with the FMax® fluorometer, using the 544 nm excitation and 612 nm emission filters. The untreated virus control and treated, infected cells were washed with PBS, fresh PBS was added, and the fluorescence was measured with the FMax® fluorometer, using the 485 nm excitation and 538 nm emission filters. The other three plates were assayed using the traditional colorimetric NR assay. After incubation for 7 days at 37° C., the cells were stained with NR following the same procedure described earlier. For the NR assay, EC₅₀ and 50% cell inhibitory concentrations (IC₅₀) were calculated by linear regression from percents of untreated, uninfected cell and untreated, infected virus controls. For the EGFP assay, EC₅₀ values were calculated by linear regression using percents of untreated, infected virus controls and IC₅₀ values were also calculated by linear regression using percents of untreated, uninfected cell control. The EC₅₀ and IC₅₀ values for each compound for both assays were compared and statistically analyzed by unpaired, two-tailed Student's t-test. A selectivity index (SI) was calculated for each compound for each assay using the formula: SI=Mean IC₅₀/Mean EC₅₀. Compounds were sorted into positive, SI≧10, and negative, SI<10, categories for the combination of NR and EGFP assays. Using the NR assay as the gold standard, sensitivity, true positives/(true positives+false negatives), and specificity, true negatives/(true negatives+false positives), were calculated.

### EXAMPLES AND EMBODIMENTS

### Example 1

### Insertion of the EGFP Gene into the HPIV-3 Antigenome and Rescue of an Infectious, Recombinant HPIV-3 Expressing the Fluorescent Protein (rHPIV3-EGFP)

A DrdI restriction site between the N gene's start signal and start codon of the HPIV-3 WT antigenome was used to facilitate the insertion of the EGFP gene. Both the forward and reverse primers that were used to amplify the EGFP open reading frame were designed to contain a DrdI restriction site on their 5′ ends. The HPIV-3 WT gene end, intercistronic, and gene start signals were encoded onto the reverse primer, resulting in an inserted EGFP gene pictured in FIG. 1, which is recognized as and behaves like an HPIV-3 WT gene. The “Rule of Six” was followed to generate an 852 by EGFP gene segment. The “Rule of Six” suggests that viral replication is most efficient when the viral genome length is a factor of six, which is likely due to a single nucleocapsid protein binding to six genomic ribonucleotides. In constructing the recombinant HPIV-3 expressing EGFP (the rHPIV3-EGFP), the presence of three G ribonucleotides, which are equally separated by five ribonucleotides starting 79 ribonucleotides from the 5′ end of the antigenome, were manipulated. This location represents one complete turn of the 3-dimensional helical structure of the nucleocapsid encased RNA genome and may co-regulate viral replication, perhaps through the assembly and binding of the viral polymerase-phosphoprotein complex with the nucleocapsids. The EGFP forward primer disrupted this natural pattern. Problems associated with the disruption were resolved by adding the three G residues in the forward primer at positions 11, 17, and 23. Before the addition of the EGFP gene segment into the antigenome, the 1-5267 cDNA segment was cloned into the pACYC177 plasmid to circumvent multiple DrdI restriction sites located in the pUC19 plasmid. The resulting 1-6119 cDNA segment, now encoding the gene for EGFP, was then cloned into the pUC19 plasmid, already containing the 5249-11366 cDNA segment. Finally, the addition of the 11284-15453 cDNA segment to the construct resulted in a complete, infectious, recombinant HPIV-3 virus, expressing the EGFP gene.

To demonstrate the successful rescue and isolation of two rHPIV3 strains, one with and one without the EGFP gene insertion, sequences surrounding three genetic markers were aligned and compared to the HPIV-3 WT virus, isolate 14702. RNA isolated from rHPIV3-EGFP, rHPIV3, and HPIV-3 WT infections was amplified by RT-PCR. The sequences generated from the 5′ RACE RT-PCR, containing the DraIII restriction site, confirmed the A to C and T to G mutations. These two mutations created a unique DraIII restriction site present only in the recombinant viruses and allowed for the insertion of the final 11284-15453 cDNA segment and completion of the recombinant viruses. The third genetic marker was also confirmed by aligning sequences generated from the 5′ end of the L gene from all three viruses. The A to G mutation eliminated one of two natural SphI restriction sites located in L gene portion of the HPIV-3 WT virus. The second SphI restriction site was used to insert both the 5249-11366 and 11284-15453 cDNA segments. These PCR products were also digested with SphI and separated on an agarose gel to show. As shown in FIG. 3, t HPIV-3 WT was digested in the presence of SphI, however, the two recombinant viruses were not.

Plaques formed by rHPIV3-EGFP were stained with crystal violet and analyzed by bright field microscopy. The viral induced syncytia absorbed more crystal violet compared to surrounding uninfected cells. The syncytia from the same plaque were visualized by fluorescent microscopy and had high concentrations of green fluorescence. On the other hand, plaques formed by HPIV-3 WT did not produce fluorescence. This result demonstrates a direct correlation between viral growth, syncytia formation, and EGFP expression.

### Example 2

### rHPIV3-EGFP Replication is Slightly Attenuated Due to The Additional Gene

The infectious virus present in the stocks of HPIV-3 WT, rHPIV3, and rHPIV3-EGFP were plaque titered and the means, ±standard deviation, of duplicate assays were found to be: 2.9±0.41×10⁷ PFU/mL for HPIV-3 WT, 2.8±0.22×10⁷ PFU/mL for rHPIV3, and 1.9±0.49×10⁷ PFU/mL for rHPIV3-EGFP. The infectious virus titer for rHPIV3 was not significantly different compared to HPIV-3 WT (p>0.01) whereas, rHPIV3-EGFP was significantly lower compared to both HPIV-3 WT and rHPIV3 (p<0.01). The addition of the EGFP gene into the HPIV-3 genome appeared to attenuate rHPIV3-EGFP compared to either the WT or recombinant strains. However, the process of creating and rescuing the recombinant virus and/or the presence of the three genetic markers did not cause attenuation of rHPIV3 because no significant reduction in virus titer was seen. For all subsequent experiments the volume of virus inoculums were adjusted so that equal PFUs were added. In addition, the replication kinetics of the three viruses, HPIV-3 WT, rHIPV3 and rHPIV-EGFP were measured to confirm the attenuation of rHPIV3-EGFP compared to the wild-type and recombinant viruses. As shown in FIG. 4, the growth curves for rHPIV3 and HPIV-3 WT were very similar, with no significant differences (p>0.01). The growth curve for rHPIV3-EGFP was significantly delayed compared to the growth curves for both HPIV-3 WT and rHPIV3 (p<0.01). During the initial stages of infection, the attenuated growth of rHPIV3-EGFP compared to both the wild-type and recombinant viruses can be seen, yet it appears that the replication of the rHPIV3-EGFP virus may recover and amplify itself to similar levels compared to the other two viruses during the later stages of replication. This result confirmed that the addition of an additional gene into the HPIV-3 genome may be the cause of attenuation.

The cytopathic effect (CPE) produced by rHPIV3-EGFP and HPIV-3 WT viruses in infected MA-104 cells was monitored for 7 days and measured by NR uptake until complete infected cell lysis occurred, verified by microscopic examination. As shown in FIG. 5, complete cell lysis induced by both viruses occurred at the same time on day 7 and no significant difference in either curve was detected (p>0.01). This result contradicted previous results showing attenuation in the replication of the rHPIV3-EGFP virus, but the result supports the idea that rHPIV3-EGFP is able to recover and replicate up to HPIV-3 WT standards.

To determine how the additional gene may have contributed to the attenuation seen during the onset of infection, a QRT-PCR assay was done to measure genomic replication and L gene transcription. An HPIV-3 specific primer that annealed to the intergenic sequence between the fusion and hemagglutinin-neuraminidase genes of the viral, negative-sense RNA, only allowing binding to viral, genomic RNA rather than viral mRNA or viral, positive-sense, anti-genomic RNA, was used as a primer for the RT reaction. An Oligo(dT)20 primer was used to prime the RT reaction for the L gene transcription measurement, binding only viral mRNA and not viral genomic RNA. Relative expression differences were calculated and normalized, using the 0 hour for each virus as the calibrator. A calibrator was used to normalize the amount of mRNA transcripts or genomic copies generated during the infections with the amount that was added at the time of infection for each virus. As shown in FIG. 6, L gene transcription, and, as shown in FIG. 7, genomic replication, were significantly reduced in an rHPIV3-EGFP infection compared to the HPIV-3 WT infection (p<0.01). The additional gene present in the HPIV-3 genome provides for a reduction in the amount of viral mRNA transcripts and genomic copies that were normally generated in a WT infection.

### Example 3

### rHPIV3-EGFP is Slightly More Sensitive to Antiviral Compounds

There is provided an rHPIV3-EGFP virus significantly more sensitive to inhibition by antiviral compounds than is the wild-type virus (p<0.05). Three antiviral compounds known to inhibit HPIV-3 were tested. The three compounds include two nucleoside analogs, ribavirin and 2-thio-6-azauridine, and a recombinant fusion protein between a sialidase catalytic domain and cell surface-anchoring sequence, DAS181. EC₅₀ values, which are the concentration of compounds that inhibit 50% of virus replication, were calculated for each compound for both HPIV-3 WT and rHPIV3-EGFP viruses. The mean, ±standard deviation, of three replicates were found to be: 35±2.5 μg/mL and 19±4.9 μg/mL for ribavirin, respectively; 1100±58 ng/mL and 630±75 ng/mL for 2-thio-6-azauridine, respectively; and 53±2.3 ng/mL and 13±2.3 ng/mL for DAS181, respectively. The rHPIV3-EGFP virus is significantly more sensitive to inhibition by these compounds than was the wild-type virus (p<0.05).

### Example 4

### Using EGFP Expression as a Measure of Viral Infectivity Leads to a Faster and More Robust Assay

Referring now to FIG. 8, there is shown data for a robust assay of viral infectivity. To determine the earliest possible day that a potential EGFP-based assay could be completed, EGFP expression by rHPIV3-EGFP was measured. The fluorescence emitted from the viral expressed EGFP was measured each day in rHPIV3-EGFP infected MA-104 cells at various MOIs of virus. EGFP expression rose in a dose-dependent manner beginning at day 1, peaked on day 3 regardless of MOI, and leveled off thereafter. Even though, the infection at MOI=1 resulted in the greatest fluorescence, a large amount of fluorescence was still detected for the other three MOIs as well. The infection at MOI=0.1 was equivalent to the concentration of virus used in typical antiviral assays, so this concentration of virus was used in further testing.

Referring now to Table 1, to compare the 3-day, EGFP-based assay to the traditional NR-based assay, Vybrant® MTT Cell Proliferation, and CellTiter-Glo® Luminescent Cell Viability Assays, the Z′-factors, signal-to-background ratios, and signal-to-noise ratios were calculated. The Z′-factor is a statistical calculation that assesses the quality of a high-throughput screening assay and predicts the potential of the assay if the number of samples were scaled up. Z′-factors were computed for each assay and compared using two different fitness tables. A higher Z′-factor value means the assay is more robust when it is used in a high-throughput format. The 3-day, EGFP-based assay, 0.83, proved to be more robust than the other three 7-day assays: 0.70 for the NR-based assay, 0.73 for the CellTiter-Glo® Luminescent Cell Viability Assay, and 0.50 for the Vybrant® MTT Cell Proliferation assay.

TABLE 1
Evaluation of the viral expressed EGFP detection method compared to three
types of viral CPE detection methods using the rHPIV3-EGFP virus.

| | | 7-Day Assay | | |
|---|---|---|---|---|
| | 3-Day Assay | | CellTiter-Glo ® | |
| | Viral expressed | Colorimetric | Luminescent Cell | Vybrant ® MTT |
| | EGFP fluorescence | neutral red uptake | Viability | Cell Proliferation |
| Z′-factor^{a} | 0.83 | 0.70 | 0.73 | 0.50 |
| Signal-to- | 241 | 65 | 6 | 7 |
| background | | | | |
| Signal-to-noise | 4057 | 301 | 59 | 60 |

^{a}The Z′-factor is a statistical calculation that assesses the quality of a high-throughput screening assay and predicts the potential of the assay if the number of samples were scaled up.

According to fitness tables, the EGFP-based, NR-based, and CellTiter-Glo® Luminescent Cell Viability Assays were all good to excellent assays. The Vybrant® MTT Cell Proliferation assay was borderline excellent/marginal on one table and at the recommended minimum level for the second table. For the signal-to-background ratios, the novel EGFP assay provided for by the invention, with a value of 241, proved to be superior showing excellent signal to background signal separation. The NR assay, value of 65, resulted in good separation, whereas both the CellTiter-Glo® Luminescent, value of 6, and Vybrant® MTT assays, value of 7, resulted in poor separation of signal to background signal. In addition, the EGFP assay, with a signal-to-noise ratio of 4057, proved to be superior to the other three assays, signal-to-noise ratios of: 301 for NR, 59 for CellTiter-Glo®, and 60 for Vybrant® MTT, by showing excellent signal to background variability separation.

### Example 5

### Comparison of a 7-Day, NR-Based Antiviral Assay and a 3-Day, EGFP-Based Antiviral Assay

Referring now to Table 2, there is shown data for rHPIV3-EGFP in an antiviral screening assay with a panel of 23 antiviral compounds. A standard 7-day, NR-based assay and a 3-day, EGFP-based assay, using the same virus stock, were done in parallel using 23 compounds, which included 22 nucleoside analogs and the one fusion protein, DAS181. A selective index (SI) value ≦3 was considered not active, SI values between 4 and 9 slightly active, between 10 and 49 moderately active, and ≧50 highly active. For purposes of this study, compounds with SI values ≧10 were considered suitable for further evaluation in additional assays. Using the threshold SI value of 10, the 3-day, EGFP-based assay had a sensitivity of 100% and specificity of 54%, compared to the 7-day NR assay. Using the 7-day NR assay as the gold standard, six compounds were falsely identified as selective inhibitors of virus replication using the rHPIV3-EGFP virus in the antiviral assay, which led to the 54% specificity. These six compounds showed an increase in the SI value over the threshold of 10 in the EGFP assay but under the threshold in the NR assay. Of these six, PSI-5449 was not active in the NR assay but was moderately active in the EGFP assay. An additional four, ribamidine, selenazofurin, PSI-5852, and PSI-5095, were considered slightly active in the NR assay and moderately active in the EGFP assay. The remaining compound, CS-1196 was considered slightly active in the NR assay and highly active in the EGFP assay. A factor that contributed to the differences in selectivity detected in each assay was the lack of toxicity found in cells exposed to compound in the EGFP assay. The toxicity of a drug is commonly determined by the concentration at which it is lethally toxic to 50% of the cells present in the assay, termed IC₅₀. No toxicity was observed for all six compounds falsely identified as selective inhibitors in the EGFP assay and the IC₅₀ values for four out of the six compounds were significantly decreased in the 7-day NR assay (p<0.05). The difference in toxicity was possibly due to the accumulation of toxicity during the 7-day incubation period of the NR assay. On the other hand, when the rHPIV3-EGFP virus was measured by the EGFP fluorescent assay the resulting EC₅₀ values were significantly lower (p<0.05) for three out of the six drugs compared to the NR assay. This result will also contribute to the higher selectivity detected for these compounds in the EGFP assay compared to the selectivity of these compounds evaluated in the NR assay. The combination of an increased IC₅₀ and a decreased EC₅₀ undoubtedly increased the SI for these six compounds and falsely suggested further evaluation, explaining the low specificity of the EGFP assay. Overall, for most other compounds a trend is seen when an

TABLE 2
Comparison of 3-Day EGFP Assay with 7-Day Colorimetric Neutral Red Uptake Assay.

| | 7-Day colorimetric neutral red uptake assay | | | 3-Day EGFP fluorescent assay | | |
|---|---|---|---|---|---|---|
| Compound Name | EC₅₀^{a} | IC₅₀^{a} | SI^{b} | EC₅₀ | IC₅₀ | SI |
| EICAR^{c} | 0.81 ± 0.061 | >100 | >120 | 0.35 ± 0.025^{d} | >100 | >280 |
| DAS181^{c} | 0.013 ± 0.0015 | >1 | >79 | 0.011 ± 0.0024 | >1 | >89 |
| PSI-5067^{e} | 0.76 ± 0.032 | 36 ± 9.2 | 48 | 0.86 ± 0.059 | >100^{d} | >120 |
| PSI-5452^{e} | 0.84 ± 0.14 | 27 ± 2.5 | 32 | 0.76 ± 0.085 | >100^{d} | >130 |
| 2-Thio-6-azauridine^{c} | 0.6 ± 0.095 | 17 ± 3.5 | 28 | 0.89 ± 0.13^{d} | >100^{d} | >110 |
| PSI-5746^{e} | 6.4 ± 0.46 | >100 | >16 | 5.1 ± 0.68 | >100 | >20 |
| CS-1164^{e} | 6.7 ± 0.21 | >94 ± 9.8 | >14 | 10 ± 3.3 | >100 | >10 |
| Ribavirin^{c} | 20 ± 3.6 | 230 ± 92 | 12 | 31 ± 2.1^{d} | >1000^{d} | >32 |
| PSI-5990^{e} | 8.7 ± 1.8 | >100 | >11 | 9.8 ± 1.9 | >100 | >10 |
| PSI-5747^{e} | 8.3 ± 2.3 | >79 ± 18 | >10 | 6.8 ± 0.46 | >100 | >15 |
| PSI-5852e | 9.9 ± 2.8 | >76 ± 21 | >8 | 2.9 ± 0.97^{d} | >100 | >35 |
| Selenazofurin^{c} | 4.9 ± 1.4 | 34 ± 7.2 | 7 | 3.5 ± 0.1 | >100^{d} | >29 |
| Ribamidine^{c} | 57 ± 15 | 390 ± 20 | 7 | 68 ± 2.6 | >1000^{d} | >15 |
| PSI-5095^{e} | 13 ± 2.1 | 83 ± 4.2 | 7 | 6.6 ± 0.53^{d} | >100^{d} | >15 |
| CS-1196^{e} | 0.5 ± 0.078 | 2.1 ± 1.1 | 4 | 0.47 ± 0.017 | >100^{d} | >210 |
| PSI-5449^{e} | 30 ± 8.2 | >95 ± 9.2 | >3 | 3.9 ± 0^{d} | >100 | >26 |
| PSI-5098^{e} | 46 ± 7 | >100 | >2 | 34 ± 0^{d} | >100 | >3 |
| CS-1227^{e} | 38 ± 4.2 | 58 ± 26 | 2 | 43 ± 2.5 | >100^{d} | >2 |
| 3-Deazaguanosine^{c} | >31 ± 1.5 | 34 ± 3.5 | 1 | 21 ± 3.2^{d} | >100^{d} | >5 |
| PSI-0194^{e} | >100 | >100 | 0 | 50 ± 4^{d} | >100 | >2 |
| CS-978^{e} | >100 | >100 | 0 | 43 ± 0.58^{d} | >100 | >2 |
| PSI-5741^{e} | >100 | >100 | 0 | >100 | >100 | 0 |
| 1-(4-methoxybenzyloxy) | >31 | 31 ± 1.2 | 0 | >41^{d} | 41 ± 2.5^{d} | 0 |
| adenosine^{c} | | | | | | |

^{a}Mean of three independent assays ± Standard Deviation;
^{b}SI = Mean IC₅₀/Mean EC₅₀;
^{c}μg/mL;
^{d}Significant difference compared to the EC₅₀ or IC₅₀ of the 7-day NR uptake assay (p < 0.05);
^{e}μM

increase in the SI value was detected in the EGFP fluorescent assay compared to the NR assay. In most cases, the increased SI can be contributed to either a significant decrease in the EC₅₀, significant increase in the IC₅₀, or a combination of both scenarios.

The present invention provides for an antiviral assay comprising the substituting rHPIV3-EGFP for HPIV-3 WT in the initial screening of potential antiviral compounds. First, attenuation of either the rHPIV3 or rHPIV3-EGFP, compared to HPIV-3 WT, was studied to determine if loss of virulence had occurred due to both the assembly and rescue of a recombinant clone or the addition of the EGFP gene. The rescued rHPIV3 virus has three genetic mutations that made it distinguishable from HPIV-3 WT. Although these three genetic markers were detected in the rescued virus, they did not attenuate the recombinant virus. Two of these markers reside in the 3′ untranslated region of the HPIV-3 antigenome and could have disrupted regulatory promoters to attenuated rHPIV3; they did not. The addition of the EGFP gene into the rHPIV3 virus, which increased the length of the viral genome by only 5% and added a seventh, distinctive gene unit, did significantly attenuate the rescued rHPIV3-EGFP virus. The attenuation was statistically significant, with a 1.5-fold reduction in rHPIV3-EGFP titers observed. The observed statistical significance may be of no practical significance because a wild-type virus grown in varying cell culture conditions: higher or lower passaged cells, confluent or less than confluent cells, variation in the media formulations, or incubation in varying conditions, may inhibit or accelerate virus replication. A 10-fold, or greater, reduction in virus titer would indicate severe attenuation and would suggest that the new virus was indeed biologically different than the wild-type strain. In support of this point, the CPE produced by rHPIV3-EGFP was not significantly inhibited or accelerated compared to the CPE produced by HPIV-3 WT throughout the duration of a viral infection. Although, decreased viral titers and slower viral replication were detected for the rHPIV3-EGFP virus, CPE produced by each virus remained substantially the same.

The attenuation of rHPIV3-EGFP may be attributed to the combination of the small increase in genome length and the addition of a foreign gene, which contributed to the significantly reduced viral genomic replication and mRNA transcription. The viral polymerase terminates and reinitiates transcription at each gene junction inconsistently, resulting in a reduction of downstream gene transcription and expression in a gradient fashion. Thus, the first of six viral genes, NP, should be expressed at significantly higher levels compared to the last L gene. Therefore, the insertion of the EGFP gene into the first viral gene position may result in a reduction in mRNA transcription in all downstream viral genes due to further inconsistent termination and reinitiation of the viral polymerase. This phenomenon was confirmed when transcription of the L gene was reduced in the rHPIV3-EGFP virus infection compared to the HPIV-3 WT virus infection. In addition, genomic replication was also reduced, perhaps due to the overall decline in the expression of necessary viral replication proteins. A significant reduction in viral transcription could also lead to a reduction in translation of the viral transcripts. Overall, less viral proteins would be available for replication purposes resulting in less efficient viral replication. Thus, less virions would be assembled because of the reduction in viral proteins and genomic RNA strands resulting in an attenuated virus. Furthermore, ribavirin and 2-thio-6-azauridine inhibit inosine monophosphate dehydrogenase and orotidine monophosphate decarboxylase, respectively, and can be classified as nucleoside analogs, which may be incorporated into the viral RNA strands and interfere with further protein translation and genome replication. The reduced EC₅₀ values are possibly related to the reduction in mRNA transcription and genomic replication; therefore, it is possible less compound is needed to incorporate into the RNA strands and inhibit viral expression. On the other hand, DAS181 eliminates the host cell receptor needed for viral entry and inhibits virion binding and absorption. The reduced EC₅₀ values are most likely due to the reduction in viable virions produced by the attenuated virus; therefore, less compound is required to prevent virion attachment. The consequence of a slightly attenuated virus is the possibility of a reduced ECₛₒ value, which may increase the SI value above the threshold of 10 and result in a false positive, meaning that the same compound may not inhibit the wild-type virus as much. This consequence is acceptable in the initial screening of a high-throughput assay because the false positive compounds would be retested in the presence of the wild-type virus and if they were true, false positives, they would be eliminated in the second round of screening.

There is provided by the present invention a faster antiviral assay. The use of a recombinant virus that expresses a reporter gene to measure viral replication leads to the possibility of detecting the virus earlier in the assay. Assays that use dyes or enzymes, like NR, Vybrant® MTT, and CellTiter-Glo® Luminescent, are only measuring the health of a cell and for these assays to be most accurate the maximum difference between signal and background needs to be achieved. This occurs for these types of assays when the virus has completely lysed the infected cell monolayer or when cell membranes have become non-functional. The length of time needed to reach this point depends upon the virus. The virus used in this study, HPIV-3, requires 7 days to achieve complete cell destruction at the MOI used. However, using an assay that measures a viral expressed reporter gene, the incubation time is only limited to when the reporter gene reaches maximal or acceptable signal-to-background ratio levels. For rHPIV3-EGFP, EGFP expression is detectable 24 hours post-infection and reaches its peak at 3 days in a dose responsive manner. The characteristic syncytia formation of the HPIV-3 virus might be the reason that abundant EGFP expression levels are achieved and remain for 5 days after the maximum expression levels are reached. Even the lowest dose of virus shown could potentially be used in the antiviral assay, but for the purpose of this study we chose to use a concentration of virus that was equivalent to the concentration of virus used in typical antiviral assays. The broad range of EGFP detection possibilities, in both the length of time and concentration of virus, could potentially be used in experiments that need more defined parameters.

The 3-day, EGFP-based assay was evaluated for use in high-throughput assays using Z′-factor analysis and other parameters. It had a good to excellent Z′-factor value in addition to very high signal-to-background and signal-to-noise ratios. The Z′-factor takes into account the variability of both the signal and background and the difference between the signal and background. Thus, the virus-expressed EGFP gene is very suitable for this type of assay because only the infected cells will fluoresce and any background measurement seen is due to autofluorescence of the plate, medium, or cells, which can be subtracted from the signal. On the other hand the NR, Vybrant® MTT, and CellTiter-Glo® luminescent assays all measure any intact cells infected or not infected with virus. This can be problematic if the virus does not lyse the cell monolayer completely because the background measurements are raised, reducing the sensitivity and validity of the assay. For example, the CellTiter-Glo® luminescent assay was very susceptible to this phenomenon because even when the cells of the virus controls were completely lysed, as determined visually, significant luminescence was measured when compared to the no-cell control (data not shown), thus the poor signal-to-background and signal-to-noise ratios for the luminescent assay.

When the 3-day, EGFP assay was evaluated with a panel of known inhibitors of HPIV-3 WT replication, the assay resulted in excellent sensitivity and marginal specificity. When the rHPIV3-EGFP virus was used in an antiviral assay and fluorescence was measured, approximately an equal number of false positives and true positives would have passed the initial round of antiviral drug screening.

Some of the differences seen between the SI values determined from the EGFP-based and NR-based assays were due to differences in drug toxicity measured at 3 and 7 days, respectively. The compounds in question seem to be less toxic on day 3 than on day 7, which implies that the toxicity effects accumulate over time. In addition, cell growth may be slowed leading to apparent cell growth inhibition due to depletion of nutrients and acid build up in the medium after 7 days of incubation. These two phenomena probably contributed to the apparent increase in toxicity as measured by the IC₅₀ values in the 7-day, NR assay. Furthermore, because of the additional factors contributing to cell toxicity a more accurate assay for detecting cell toxicity is conducted in follow-up studies for active compounds using rapidly-dividing MA-104 cells, which are incubated for 3 days in the absence of virus and measured by NR uptake. In essence, the 3-day, EGFP assay may be more accurate compared to the 7-day, NR assay for measuring cell toxicity.

The development of the rHPIV3-EGFP virus and its use in antiviral testing has increased the sensitivity and quality of an HPIV-3 antiviral assay that measures EGFP fluorescence. The EGFP assay has shortened the duration and significantly decreased the time consuming and labor intensive nature of the NR dye uptake assay. Overall, the use of the rHPIV3-EGFP virus in initial antiviral drug testing reduces the amount of time needed to obtain results and may be beneficial when testing numerous compounds in a high-throughput format. These conclusions warrant the replacement of the HPIV-3 WT virus with the rHPIV3-EGFP virus in initial antiviral testing. Finally, additional research to improve the 3-day, EGFP assay might include scaling-up to a 384-well plate format and the development of a non-green fluorescent dye to replace NR in cell toxicity measurements.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

### Example 6

### Construction of a Full-Length Recombinant HPIV-3 Clone Containing the EGFP Gene (rHPIV3-EGFP)

The following disclosure describes in a procedure used to amplify and assemble three viral antigenomic cDNA segments encompassing the entire HPIV-3 antigenome. It also describes the insertion of the EGFP gene into the HPIV-3 antigenome as a distinct transcription unit. The DrdI restriction site was chosen as the site for inserting the EGFP gene because of its prime location upstream of the first gene's start codon. To circumvent the additional DrdI sites located in the pUC19 parent vector, the pACYC177 plasmid was used as the backbone for the insertion of the EGFP gene into the HPIV-3 antigenome.

This disclosure also describes the insertion of a customized polylinker, which contains the necessary restriction sites, the final 28 nucleotides of the HPIV-3 antigenome, a hepatitis delta ribozyme, and a T7 transcription termination signal, into the parent vector to facilitate the assembly of the complete antigenome. The first two viral antigenomic cDNA segments, 5.3-kb and 6.1-kb, can be added to the polylinker/parent plasmid in any order. However, the antigenomic 4.2-kb cDNA segment needs to be the last segment added to the polylinker/parent plasmid because it is also cut with the PacI enzyme used to clone the 5.3-kb segment, and this will interfere with proper alignment of the antigenomic segments.

### Materials

HPIV-3 virus (e.g., Strain 14702), MA-104 cells (ATCC), QIAamp viral RNA mini kit (Qiagen) ProSTAR First-Strand RT-PCR kit (Stratagene).
Primers (see Table 15F.1.1 for sequence details): 5.3-kb forward, 5.3-kb reverse, 6.1-kb forward, 6.1-kb reverse, 4.2-kb forward, 4.2-kb reverse, M13/pUC sequencing primer (−40) (NEB), M13/pUC reverse sequencing primer (−48) (NEB), 6.1-kb Mut-forward, 6.1-kb Mut-reverse, EGFP-forward, EGFP-reverse, Term-forward, Term-reverse, Rib-forward, Rib-reverse.

### Enzymes:

PfuTurbo Hotstart DNA polymerase (Stratagene), T4 DNA ligase (NEB), T4 DNA polymerase (NEB), Sequenase version 2.0 DNA polymerase (USB), Calf intestine alkaline phosphatase (CIP; NEB).
2-Log DNA ladder (NEB), QIAEX II gel extraction kit (Qiagen), QIAquick PCR purification kit (Qiagen), Plasmids, pUC19 (NEB), pEGFP (BD Biosciences Clontech), pACYC177 (NEB), Restriction enzymes (NEB), SmaI, AatII, BstEII, DrdI, KpnI, DraIII, SphI, PacI, Electrocomp GeneHogs E. coli (Invitrogen), imMedia Amp Blue (Invitrogen), imMedia Amp liquid (Invitrogen), QIAprep Spin miniprep kit (Qiagen), QuikChange XL site-directed mutagenesis (Stratagene), imMedia Amp Agar (Invitrogen), Subcloning efficiency DH5α chemically competent E. coli (Invitrogen), TE buffer, EndoFree plasmid maxi kit (Qiagen), 0.1-ml thin-walled PCR tubes (BioRad), Thermal cycler (e.g., GENEMate), 37° and 60° and 65° C. water baths, Electroporation apparatus (e.g., Gene Pulser, Bio-Rad), 37° C. incubators (rotating and non-rotating) Sterile 14-ml snap-cap culture tubes (Fisher) Additional reagents and equipment for performing agarose gel electrophoresis.

### RT-PCR Amplify HPIV-3 Antigenomic Segments

1. Infect MA-104 cells with an HPIV-3 strain. Purify viral RNA from the clarified supernatant of HPIV-3-infected MA-104 cells using the QIAamp viral RNA mini kit following the manufacturer's instructions, with no modifications. HPIV-3 strain 14702 was used as a source of viral RNA for cDNA synthesis, although other HPIV-3 strains may be substituted.
2. Synthesize three HPIV-3 antigenomic cDNA segments, 5.3-, 6.1-, and 4.2-kb, using the ProSTAR First-Strand RT-PCR kit, 300 ng of each of the forward primers (Table 3), and purified HPIV-3 viral RNA in 0.1-ml thin-walled PCR tubes in a thermal cycler according to the manufacturer's disclosure. These primers were designed from a consensus of antigenomic sequences of three other HPIV-3 strains, JS, 47885, and GPv. Other HPIV-3-specific primers may be used to incorporate other promoters and/or restriction sites. Alternatively, other first-strand RT-PCR kits common in the art may be used.
3. Amplify each antigenomic cDNA segment using PfuTurbo Hotstart DNA polymerase and 120 ng of both forward and reverse primers (Table 3) in 0.1-ml thinwalled PCR tubes in a thermal cycler following the manufacturer's disclosure with the following exceptions: 30 cycles and annealing for 6 min at 50° C. (for 5.3-kb and 6.1-kb segments) or 6 min at 48.0 (for 4.2-kb segment).
During this step, use a high-fidelity proofreading DNA polymerase to reduce the number of mutations, which may be lethal to the recombinant virus. Even though this disclosure uses the PfuTurbo Hotstart DNA polymerase, other high-fidelity proofreading DNA polymerases may be used. The use of a proofreading DNA polymerase during amplification will generate blunt ends that will allow cloning of PCR products into any restriction site cut with a restriction endonuclease generating blunt ends.
4. Check for the presence and correct length of each antigenomic cDNA segment by using a 0.8% agarose gel and 2-Log DNA marker for assessing DNA length.
5. If multiple bands exist in any reaction, excise and purify the bands of correct length with the QIAEX II gel extraction kit. Otherwise, purify the PCR product with the QIAquick PCR purification kit if only one band is seen during gel electrophoresis.

### Clone Antigenomic Segments

6. Linearize pUC19 with SmaI at room temperature according to the manufacturer's disclosure.
7. Purify the pUC19 digestion with the QIAquick PCR purification kit.
8. Ligate each purified antigenomic cDNA PCR product into the digested pUC19 vector using T4 DNA ligase according to the manufacturer's disclosure.
9. Heat-inactivate the T4 DNA ligase for 15 min in a 65° C. water bath.
Alternatively, the ligated DNA may be purified with the QIAEX II gel extraction kit for optimal transformation efficiency. Heat inactivation of the ligase enzyme results in increased transformation efficiencies for ligated DNA compared to untreated, ligated DNA but lower transformation efficiencies for ligated DNA compared to purified, ligated DNA.
10. Electroporate the ligated DNA into Electrocomp GeneHogs E. coli at 1.6 kV, 25 μF, and 200Ω according to the manufacturer's disclosure using an electroporation apparatus. High efficiency transformation was achieved with electroporation; however, other transformation methods could be used.
11. Spread transformants onto imMedia Amp Blue agar plates and incubate overnight in a 37° C. incubator.
12. Select several white bacterial colonies, inoculate into 3 ml imMedia Amp liquid cultures in sterile 14-ml snap-cap culture tubes, and incubate overnight in a 37° C. rotating incubator.
Invitrogen's imMedia was used for reliability and convenience, although traditional LB medium may also be used. Bacterial stocks can also be made by adding sterile glycerol, 17% final volume, and freezing at −80° C.
13. Isolate DNA plasmids from each culture using the QIAprep Spin miniprep kit.
14. Screen the DNA of several clones for the presence of each antigenomic cDNA insert by restriction digestion and gel electrophoresis.
15. Sequence positive clones starting with the M13/pUC sequencing primer (−40) and M13/pUC reverse sequencing primer (−48) and continue sequencing the complete cDNA insert with virus-specific primers in both directions.
The resulting positive clones were named 5.3-kb, 6.1-kb, and 4.2-kb, which represent the length of each PCR product. The final order of each antigenomic cDNA segment in the full-length clone is 5.3-kb, 6.1-kb, and 4.2-kb. The complete antigenomic sequence for HPIV-3 strain 14702 can be found in Genbank, accession no. EU424062.
16. Mutate A-to-G, located at viral nucleotide position 8635, in the antigenomic 6.1-kb cDNA segment to eliminate a second SphI restriction site using QuikChange XL site-directed mutagenesis, following the manufacturer's disclosure.
The native antigenomic 6.1-kb cDNA segment of HPIV-3, strain 14702 has two SphI restriction sites. Therefore, the SphI restriction site located in the middle of the 6.1-kb segment must be eliminated to avoid interference with further subsequent cloning. Other HPIV-3 strains may not have this undesirable SphI restriction site.

TABLE 3
Primers used in the cloning of the rHPIV-EGFP cDNA Clone

| Event/primer | Sequences(5′ to 3′) | | Highlights |
|---|---|---|---|
| Viral antigenomic | | | |
| cDNA synthesis | | | |
| 5.3-kb-forward | SEQ ID NO: 11 | CCGACGTCTTAATTAATACGACTCACT | Bold: AatII and PacI |
| | | ATAGGACCAAACAAGAGAAGAAACTT | restriction sites |
| | | | Underlined: T7 |
| | | | promoter sequence |
| 5.3-kb-reverse | SEQ ID NO: 12 | GGTCACCACAAGAGTTAGA | Bold: natural BstEII |
| | | | restriction site |
| 6.1-kb-forward | SEQ ID NO: 13 | TCTAACTCTTGTGGTGACC | Bold: natural BstEII |
| | | | restriction site |
| 6.1-kb-reverse | SEQ ID NO: 14 | ATTCATCCCAAGGGCAATA | |
| 4.2-kb-forward | SEQ ID NO: 15 | AGAATGGTTATTCACCTGTTC | |
| 4.2-kb-reverse | SEQ ID NO: 16 | GAGAAGCACTCTGTGTGGTAT | Bold: mutated DraIII |
| | | | restriction site |
| | | | Mutations underlined: A |
| | | | to C and T to G |
| Site-directed | | | |
| mutagenesis | | | |
| 6.1-kb Mut-forward | SEQ ID NO: 19 | CTTAGGAGCAAAGCGTGCTCAG | Bold: A to G mutation |
| | | AAAATGGACACTG | |
| 6.1-kb Mut reverse | SEQ ID NO: 18 | CAGTGTCCATTTTCTGAGCACGC | Reverse complement |
| | | TTTGCTCCTAAG | of 6.1-kb Mut-forward |
| EGFP gene | | | |
| amplification | | | |
| EGFP forward | SEQ ID NO: 28 | TTGACTAGAAGGTCAAGAACC | Bold: DrdI restriction |
| | | TGCAGGTCGACTCTAGAGGAT | site |
| EGFP reverse | SEQ ID NO: 29 | TTGACCTTCTAGTCAATGT | Bold: DrdI restriction |
| | | CTTTAATCCTAAGTTTTTCTTATTT | site Underlined: |
| | | ATTAACCGGCGCTCAGTTGGAAT | HPIV-3 gene |
| | | | transcriptional end, |
| | | | intercistronic, and |
| | | | gene transcriptional |
| | | | start signals |
| Customized | | | |
| Polylinkers | | | |
| Term forward | SEQ ID NO: 22 | TTTTTGTGCGCCCAATACGCAAACCGCC | Italics: Vaccinia virus |
| | | TCTCCCCGCGCGTTGGCCGTTAATTAA | termination sequence |
| | | GAGGGTGACCCTGCACAGAGTGCC | Bold: PacI, BstEII, and |
| | | | DraIII restriction sites |
| Term reverse | SEQ ID NO: 23 | TTTTTGTAAAAAACCCCTCAAGACCCGTTT | Italics: Vaccinia virus |
| | | AGAGGCCCCAAGGGGTTATGCTAGTTA | termination sequence |
| | | GGTACCCGGGCACTCTGTGCAG | Underlined: T7 |
| | | | termination sequence |
| | | | Bold: |
| | | | KpnI, SmaI, and DraIII |
| | | | restriction sites |
| Rib-forward | SEQ ID NO: 24 | ACCA [symbol] CTTCTCTTGTTTGGT | Bold: DraIII restriction |
| | | GGGTCGGCATGGCATCTCCACCTCC | site Italics: Final 28 |
| | | TCGCGGTCCGACCT | nucleotides of the |
| | | | HPIV-3 antigenome |
| | | | Underlined: |
| | | | Antigenomic hepatitis |
| | | | delta virus |
| | | | ribozyme sequence |
| Rib-reverse | SEQ ID NO: 25 | GGCCGGTACCTCCCTTAGCCATCCGAGTG | Bold: KpnI restriction |
| | | GACGACGTCCTCCTTCGGATGCCCAGG | site Underlined: |
| | | TCGGACCGCGA | Antigenomic hepatitis |
| | | | delta virus ribozyme |
| | | | sequence |
| SphI adapter | | | |
| Adapter-forward | SEQ ID NO: 26 | GTGACCGCGCATGCCCACAGA | Bold: SphI restricton |
| | | | site Underlined: BstEII |
| | | | and DraIII restriction |
| | | | sites |
| Adapter-reverse | SEQ ID NO: 27 | GTGGGCATGCGCG | Bold: SphI restricton |
| | | | site Underlined: BstEII |
| | | | and DraIII restriction |
| | | | sites |

### PCR-Amplify EGFP ORF

17. PCR amplify the open reading frame of EGFP in 0.1-ml thin-walled PCR tubes in a thermal cycler using the PfuTurbo Hotstart DNA polymerase enzyme following the manufacturer's disclosure. Use 1 ng pEGFP plasmid as template; 20 μM EGFP forward And 20 μM EGFP-reverse (Table 3) as primers; and change the cycling parameters as follows: 58° C. for the annealing temperature, 1 min for the extension time, and use 30 cycles. To abide by the “Rule of Six,” the primers used to amplify the EGFP ORF were designed to generate a PCR product that results in an 852-bp band, a factor of six, when digested with DrdI in later steps. In addition, three equally spaced G nucleotides were added to the forward primer at positions 11, 17, and 23 to restore a natural bipartite replication promoter on the 3′ end of the viral genome.
18. Repeat steps 4 through 15 to clone the resulting 868-bp band, representing the PCR-amplified EGFP ORF into a naive pUC19 vector.
Clone EGFP into the 5.3-Kb Antigenomic cDNA Segment
19. Digest the pACYC 177 plasmid and the plasmid containing the antigenomic 5.3-kb cDNA segment with AatII and BstEII restriction enzymes, sequentially, in 37° C. and 60° C. water baths, respectively, according to the manufacturer's disclosure.
20. Separate both digestions, individually, by gel electrophoresis and purify the ˜5.0-kb band, representing the antigenomic 5.3-kb cDNA segment, and the ˜4.0-kb band, representing the pACYC 177 plasmid, using the QIAEX II gel extraction kit.
21. Ligate the purified antigenomic 5.3-kb cDNA segment into the purified pACYC177 vector using T4 DNA ligase according to manufacturer's disclosure.
The addition of the EGFP gene into the antigenomic 5.3-kb cDNA segment uses the DrdI restriction site. The parent plasmid pUC19 cuts two times with DrdI, so it is necessary to transfer the 5.3-kb cDNA segment into a second plasmid that does not contain additional DrdI sites. The pACYC 177 contains one DrdI restriction site located on a small 284-bp segment between AatII and BstEII restriction sites, which is eliminated during the purification of the larger 4.0-kb segment from the smaller 284-bp segment resulting from the AatII/BstEII digestion. Other vectors, which do not contain DrdI sites, may also be used.
22. Heat-inactivate the T4 DNA ligase 15 min in a 65° C. water bath.
23. Electroporate the ligated DNA into Electrocomp GeneHogs E. coli at 1.6 kV, 25 μF, and 200Ω, according to the manufacturer's disclosure using an electroporation apparatus.
24. Spread transformants onto imMedia Amp Agar plates and incubate overnight in a 37° C. incubator.
25. Select several bacterial colonies, inoculate into 5 ml imMedia Amp liquid, and incubate cultures overnight in a 37° C. rotating incubator.
The pACYC177 plasmid is a low-copy number vector; therefore, little to no DNA may be obtained using traditional methods. Sufficient DNA can purified for cloning purposes by performing DNA isolation on the entire 5-ml culture in three separate 1.5-ml preparations.
26. Isolate DNA plasmids from each culture using the QIAprep Spin miniprep kit.
To concentrate and purify more vector DNA, apply the supernatants from three bacterial lysates to one column, allowing each supernatant to flow through the column first before loading the subsequent supernatants.
27. Screen the DNA of several clones for the presence of the antigenomic 5.3-kb cDNA insert in the pACYC177 backbone by restriction digestion, gel electrophoresis, and DNA sequencing.
28. Digest the plasmids containing the PCR-amplified EGFP ORF and the pACYC177/5.3-kb plasmid with DrdI in a 37° C. water bath according to the manufacturer's disclosure.
29. Dephosphorylate the ends of the pACYC177/5.3-kb plasmid with CIP in a 37° C. water bath according to the manufacturer's disclosure.
30. Separate the PCR-amplified EGFP ORF digestion by gel electrophoresis and purify the 852-bp band, representing the EGFP ORF, using the QIAEX II gel extraction kit.
31. Repeat steps 21 through 27 to clone the EGFP ORF into the antigenomic 5.3-kb cDNA segment.
The DrdI restriction site is non-palindromic; therefore, directional cloning should occur using only one restriction enzyme.
Eliminate KpnI Site from Parent Vector
32. Linearize the raw pUC19, containing no insert, with KpnI in a 37° C. water bath according to the manufacturer's disclosure.
The native KpnI restriction site located in the pUC19 multiple cloning site must be eliminated because a KpnI restriction site is reintroduced and used in later cloning steps. The pUC19 parent vector was selected and used because of the lack of certain restriction sites that are used in downstream applications. Other cloning vectors are commercially available and could also be used.
33. Blunt the 3′ overhang ends, generated by KpnI cleavage, using T4 DNA polymerase following manufacturer's disclosure.
34. Purify the reaction with the QIAquick PCR purification kit.
35. Recircularize the plasmid with T4 DNA ligase, according to the manufacturer's disclosure.
36. Transform the ligated DNA into subcloning efficiency DH5α chemically competent E. coli following manufacturer's disclosure.
37. Spread transformants onto imMedia Amp Blue agar plates and incubate overnight in a 37° C. incubator.
38. Select several white bacterial colonies, inoculate into 3 ml imMedia Amp liquid cultures, and incubate overnight in a 37° C. rotating incubator.
39. Isolate DNA plasmids from each culture using the QIAprep Spin miniprep kit.
40. Screen DNA of several clones for the presence of the SmaI restriction site by restriction digestion and gel electrophoresis.
The KpnI and SmaI restriction sites overlap each other in the pUC19 multiple cloning site. Clones that have the typical four nucleotide deletions also eliminate the SmaI site. On the other hand, clones that have five deletions may leave the SmaI restriction site intact.
41. Confirm the elimination of the KpnI restriction site and presence of the SmaI site by DNA sequencing.
The resulting plasmid was named pUC19-T.

### Add Customized Polylinkers to Parent Vector

42. Separately heat 1 μg of the forward and reverse oligonucleotides for Term and Rib (Table 3) 10 min to 70° C. in TE buffer.
The forward and reverse primers for both Term and Rib contain a 14-nucleotide overlap on their 3′ ends so they can anneal to each other.
43. Slowly cool the mixture to 37° C.
44. Extend each oligonucleotide with Sequenase version 2.0 DNA polymerase, according to the manufacturer's disclosure.
As long as the primers are annealed properly, secondary structure is of no concern. The Sequenase enzyme ignores secondary structure during elongation. Elongation should occur at each 3′ end using the opposite oligonucleotide as template.
45. Purify the small double-stranded DNA products with QIAEX II gel extraction kit.
46. Blunt the ends of the small double-stranded DNA products using T4 DNA polymerase following manufacturer's disclosure.
47. Purify the products a second time with QIAEX II gel extraction kit.
48. Digest pUC19-T with SmaI at room temperature according to the manufacturer's disclosure.
49. Ligate the purified small double-stranded DNA products Term and Rib into the digested pUC19-T, separately, using T4 DNA ligase according to the manufacturer's disclosure.
50. Transform the ligated DNA into subcloning efficiency DH5α chemically competent E. coli following manufacturer's disclosure.
51. Spread transformants onto imMedia Amp agar plates and incubate overnight in a 37° C. incubator.
52. Select several bacterial colonies, inoculate into 3 ml imMedia Amp liquid cultures, and incubate overnight in a 37° C. rotating incubator.
53. Isolate DNA plasmids from each culture using the QIAprep Spin miniprep kit.
54. Screen the DNA of several clones for the presence of the small double-stranded DNA inserts by restriction digestion and gel electrophoresis.
55. Confirm the presence and validate the sequence of each insert by DNA sequencing. The plasmid that contains the Term segment was named pUC19-A and the plasmid that contains the Rib segment was named pUC19-R.
56. Digest the pUC19-A and pUC19-R plasmids with DraIII and KpnI restriction enzymes, sequentially, in a 37° C. water bath according to the manufacturer's disclosure.
57. Separate the pUC19-R DraIII/KpnI digestion by gel electrophoresis and purify the 108-bp band with QIAEX II gel extraction kit.
58. Repeat steps 49 through 55 to clone the purified Rib 108-bp insert into pUC19-A.
The resulting plasmid was named pUC19-B.

### Realign SphI Restriction Site

59. Linearize pUC19-B with the SphI restriction enzyme in a 37° C. water bath according to the manufacturer's disclosure.
60. Repeat steps 33 through 41 to eliminate the native SphI restriction site.
The resulting plasmid was named pUC19-C.
61. Digest pUC19-C with DraIII and BstEII restriction enzymes, sequentially, in 37° C. and 60° C. water baths, respectively, according to the manufacturer's disclosure.
62. Repeat steps 42 and 43 to anneal the forward and reverse SphI adapter primers together (Table 3).

63. Repeat steps 49 through 55 to clone the adaptor into the digested pUC19-C.

The resulting plasmid was named pUC19-D.

### Assemble Full-Length Antigenome

64. Digest pUC19-D and the mutated antigenomic 6.1-kb cDNA segment with SphI and BstEII, sequentially, in 37° C. and 60° C. water baths, respectively, according to the manufacturer's disclosure.
65. Separate the mutated antigenomic 6.1-kb cDNA segment digestion by gel electrophoresis and purify the ˜6-kb band using the QIAEX II gel extraction kit.
66. Ligate the purified mutated antigenomic 6.1-kb cDNA segment into the digested pUC19-D using T4 DNA ligase according to the manufacturer's disclosure.
67. Heat-inactivate the T4 DNA ligase 15 min in a 65° C. water bath.
68. Electroporate the ligated DNA into Electrocomp GeneHogs E. coli at 1.6 kV, 25 μF, and 200Ω according to the manufacturer's disclosure using an electroporation apparatus.
69. Spread transformants onto imMedia Amp Agar plates and incubate overnight in a 37° C. incubator.
70. Select several bacterial colonies, inoculate into 3 ml imMedia Amp liquid cultures, and incubate overnight in a 37° C. rotating incubator.
71. Isolate DNA plasmids from each culture using the QIAprep Spin miniprep kit.
72. Screen the DNA of several clones for the presence of the mutated antigenomic 6.1-kb cDNA insert by restriction digestion, gel electrophoresis, and DNA sequencing.
The resulting plasmid was named pUC19-F.
73. Sequentially digest pUC19-F and the antigenomic 5.3-kb cDNA segment containing the EGFP gene with PacI followed by BstEII in 37° C. and 60° C. water baths, respectively, according to the manufacturer's disclosure.
74. Separate the digested antigenomic 5.3-kb cDNA/EGFP segment by gel electrophoresis and purify the ˜5-kb band using the QIAEX II gel extraction kit.
75. Repeat steps 66 through 72 to clone the 5.3-kbcDNA/EGFP segment into the digested pUC19-F.
The resulting plasmid was named pUC19-I. The addition of either the 5.3-kb or 6.1-kb antigenomic cDNA segment can occur in any order. However, the final addition of the 4.2-kb antigenomic cDNA segment needs to occur last.
76. Sequentially digest pUC19-I and the antigenomic 4.2-kb cDNA segment with DraIII and SphI, in a 37° C. water bath according to the manufacturer's disclosure.
77. Separate the antigenomic 4.2-kb cDNA segment digestion by gel electrophoresis and purify the ˜4-kb band using the QIAEX II gel extraction kit.
78. Repeat steps 66 through 72 to clone the 4.2-kb cDNA segment into the digested pUC19-I.
The resulting plasmid was named pUC19-J, which represents the full-length recombinant HPIV-3 cDNA clone expressing EGFP.
79. Purify transfection quality pUC19-J plasmid DNA using the EndoFree plasmid maxi kit following the manufacturer's disclosure. Store for at least 2 years at −80° C.

### Example 7

### Cloning of HPIV-3 Support Genes

The following disclosure describes the amplification and cloning of three HPIV-3 genes that code for the nucleocapsid protein (NP), phosphoprotein (P), and large protein (L), all of which are necessary for viral replication and transcription. The presence of these proteins during the rescue of the recombinant virus is necessary to replicate and transcribe the rHPIV-3 viral RNA to stimulate a productive infection. The transcription of these genes from plasmids is initiated by a T7 promoter, which is similar to the promoter initiating the transcription of the full-length antigenomic cDNA but is part of the commercially available pTNT plasmid from Promega. To successfully express these proteins, the orientation of the genes in relation to the T7 promoters is crucial. The start codon for each gene should be placed downstream of the T7 promoter. The T7 DNA polymerase used to transcribe these viral genes is supplied from a recombinant vaccinia virus discussed in Basic Disclosure 3.

### Materials

Template (see Table 4); Primers (20 μM; see Table 4 for sequence details):

### NP-forward, NP-reverse

| | Nucleocapsid gene (NP) | | Phosphoprotein gene (P)^{a} | | Large protein gene (L) | |
|---|---|---|---|---|---|---|
| Forward primer (5′ to 3′) | SEQ ID NO: 30 | | SEQ ID NO: 32 | | SEQ ID NO: 34 | |
| Reverse primer (3′ to 5′) | SEQ ID NO: 31 | | SEQ ID NO: 33 | | SEQ ID NO: 35 | |
| Primer concentration | 20 | μM | 20 | μM | 20 | μM |
| Template | Antigenomic 5.3-kb cDNA | | Antigenomic 5.3-kb cDNA | | pUC19-J | |
| | segment | | segment | | | |
| Annealing Temperature | 51° | C. | 51° | C. | 51° | C. |
| Extension Time | 2 | minutes | 2 | minutes | 7 | minutes |
| Cycles | 30 | | 30 | | 30 | |
| Approximate Size | 1.5 | kb | 1.8 | kb | 7.0 | kb |

### PCR Amplify Viral Support Genes

1. Amplify the open reading frames of the viral NP, P, and L genes by PCR in 0.1-ml thinwalled PCR tubes in a thermal cycler using the PfuTurbo Hotstart DNA polymerase enzyme and following the manufacturer's disclosure. Use the experimental conditions found in Table 4.
2. Check for the presence and correct length of each antigenomic cDNA segment by gel electrophoresis.
3. Purify the PCR products with the QIAquick PCR purification kit.
Clone Viral Support Genes into pUC19
4. Linearize pUC19 with SmaI at room temperature according to manufacturer's disclosure.
5. Purify the pUC19 digestion with the QIAquick PCR purification kit.
6. Ligate the PCR products for each purified support gene into the digested pUC19 vector using T4 DNA ligase according to manufacturer's disclosure.
7. Transform the ligated DNA for the NP and P clones into subcloning efficiency DH5α chemically competent E. coli following manufacturer's disclosure.
The NP and P clones were transformed into DH5α E. coli because of the size of the inserts, which are ˜1.5 kb and 1.8 kb, respectively.
8. Heat-inactivate the T4 DNA ligase used to ligate the DNA for the L clone 15 min in a 65° C. water bath.
9. Electroporate the ligated DNA for the L clone into Electrocomp GeneHogs E. coli at 1.6 kV, 25 μF, and 200Ω according to the manufacturer's disclosure using an electroporation apparatus. The L clones were electroporated into GeneHogs because of the size of the insert, ˜7 kb.
10. Spread transformants for all three support genes onto imMedia Amp Blue agar plates and incubate overnight in a 37° C. incubator.
11. Select several white bacterial colonies, inoculate into 3 ml imMedia Amp liquid cultures, and incubate overnight in a 37° C. rotating incubator.
12. Isolate DNA plasmids from each culture using the QIAprep Spin miniprep kit.
13. Screen the DNA of several clones for the presence of each viral support gene insert by restriction digestion and gel electrophoresis.
14. Sequence positive clones starting with the M13/pUC sequencing primer (−40) and M13/pUC reverse sequencing primer (−48) and continue sequencing the complete support gene insert with gene-specific primers in both directions.
The resulting positive clones were named pUC19-NP, P, or L. The insertion of each support gene into pUC19 occurred bi-directionally. Screen and select clones whose orientation resulted in the gene's start codon downstream of the KpnI restriction site, not the SalI restriction site. When each support gene is directionally cloned into the pTNT vector in the next step, the T7 polymerase will drive the transcription of the support gene only when properly oriented.
Clone Viral Support Genes into T7 Expression Plasmid
15. Digest the pTNT plasmid and the plasmids containing the NP, P, and L support genes with KpnI and SalI restriction enzymes, sequentially, in a 37° C. water bath according to the manufacturer's disclosure.
16. Separate support gene digestions, individually, by gel electrophoresis and purify the ˜1.5-kb band, representing the NP gene, the ˜1.8-kb band, representing the P support gene, and the ˜7.0-kb band, representing the L support gene, using the QIAEX II gel extraction kit.
17. Repeat steps 6 through 9 to ligate and transform the purified support genes into the digested pTNT plasmid.
18. Spread transformants for all three support genes onto imMedia Amp agar plates and incubate overnight in a 37° C. incubator.
19. Select several bacterial colonies, inoculate into 3 ml imMedia Amp liquid cultures, and incubate overnight in a 37° C. rotating incubator.
20. Isolate DNA plasmids from each culture using the QIAprep Spin miniprep kit.
21. Screen the DNA of several clones for the presence of each viral support gene insert by restriction digestion, gel electrophoresis, and DNA sequencing.
The resulting plasmids were named pTNT-NP, P, and L.
22. Purify transfection-quality pTNT-NP, P, and L plasmid DNA using the EndoFree plasmid maxi kit following the manufacturer's disclosure. Store for at least 2 years at −80° C.

### Example 8

### Rescuing Infectious, Recombinant HPIV-3 Viruses

The nucleotide sequence of SEQ ID NO: 41 is a completed cDNA clone constructed and used for the rescue of an infectious, recombinant HPIV-3 virus. Those in the art would recognize that substantially similar sequences, including, but not limited to, nucleotide sequences at least 95%, or at least 98%, or at least 100% identical to SEQ ID NO: 41 could be produced by standard laboratory techniques and used in methods substantially similar to those employed in the use of SEQ ID NO: 41 as described herein. SEQ ID NO: 42 is an RNA version of a rescued recombinant human parainfluenza virus, and is an example nucleotide sequence for a positive-sense antigenome that can be rescued by the methods described herein. One in the art would recognize that a change in the cDNA clone constructed and used for the rescue of an infectious, recombinant HPIV-3 virus, would result in a corresponding change in the negative sense genomic RNA of the rescued virus

The following describes the process of rescuing an infectious rHPIV-3 virus from a full-length antigenomic cDNA clone. The recombinant vaccinia virus, vTF7-3, expresses a T7 DNA polymerase, which transcribes the full-length viral antigenomic cDNA and the three support plasmids. The mRNAs for the nucleocapsid protein, phosphoprotein, and large protein are further translated into proteins that replicate and transcribe the full-length viral genomic RNA, resulting in the assembly of infectious rHPIV-3 virions. To minimize the replication of the recombinant vaccinia virus, Ara-C is added to the medium, which inhibits DNA replication. Subsequently, plaque purifications are also done to further remove residual vTF7-3 particles and prevent contamination of rHPIV-3 stocks.

### Materials

HeLa cells (ATCC #CCL-2)
Minimum essential medium with Earle's balanced salts (MEM; Hyclone, cat. no. SH30024.02)
Standard fetal bovine serum (FBS; Hyclone, cat. no. SH30088.03)
10 mM non-essential amino acids solution in MEM (NEAA; Invitrogen, cat. no. 11140050)
100 mM sodium pyruvate solution in MEM (Invitrogen, cat. no. 11360070) vTF7-3
Opti-MEM I reduced-serum medium (Gibco, cat. no. 11058-021)

### Plasmids:

pUC19-J
pTNT-NP
pTNT-P
pTNT-L
Lipofectamine 2000 transfection reagent (Invitrogen, cat. no. 11668019)
Cytosine β-D-arabinofuranoside (Ara-C; Sigma, cat. no. C1768)
MA-104 cells (ATCC)
2% agarose

### 2×MEM

12-well plates (Costar no. 3513, Corning)
Water-jacketed, 37° C., 5% CO2 humidified incubator (e.g., Isotemp, Thermo Fisher Scientific)
Cell scrapers (Fisher Scientific, cat. no. 08-773-3)
25-cm2 flasks
1-ml pipets

### Transfect Cells

1. Seed HeLa cells in a 12-well plate at 8×105 cells/well in MEM supplemented with 10% FBS, 0.1 mM NEAA, and 1 mM sodium pyruvate.
2. Incubate HeLa cells overnight in a water-jacketed, 37° C. and 5% CO2 humidified incubator.
3. Replace growth medium with 500 μl MEM supplemented with 2% FBS, 0.1 mM NEAA, and 1 mM sodium pyruvate.
4. Infect HeLa cells with vTF7-3 at a concentration of 5.4×105 plaque forming units (pfu)/cell or 1 multiplicity of infection (MOI).
5. Incubate infected cells 1 hr in a 37° C., 5% CO2 humidified incubator.
6. Remove virus/medium mixture and add 400 μl of Opti-MEM I supplemented with 0.1 mM NEAA.
7. Transfect infected HeLa cells with 0.4 μg pUC19-J, 0.8 μg pTNT-NP, 1.6 μg pTNTP, and 0.04 μg pTNT-L, and 5.3 μl of Lipofectamine 2000 according to the manufacturer's disclosure.
8. Incubate transfected cells 4 to 5 hr in a 37° C., 5% CO2 humidified incubator.
9. Add 500 μl of MEM supplemented with 20% FBS, 0.1 mM NEAA, 1 mM sodium pyruvate, and 250 μg/ml of Ara-C to the transfected cells.
10. Incubate transfected cells 48 hr in a 37° C., 5% CO2 humidified incubator.
11. Scrape transfected cells off the plate with a sterile cell scraper and freeze the cell suspension in 10% glycerol for at least 2 years at −80° C.
Typical HPIV-3-induced cytopathic effect (CPE) cannot be seen at the conclusion of this step. However, most cell death that is observed is due to vTF7-3-induced CPE, which is characterized by cellular rounding and sloughing, even though the Ara-C inhibitor is present in the medium.
Amplify Infectious rHPIV3-EGFP
12. Seed 3×106 MA-104 cells in a 25-cm2 flask in MEM supplemented with 10% FBS.
13. Incubate the MA-104 cells overnight in a 37° C., 5% CO2 humidified incubator.
14. Remove the growth medium and add 800 μl MEM.
15. Rapidly thaw HeLa cells by swirling in a 37° C. water bath. Add 200 μl of the transfected HeLa cell lysate containing recombinant virus to the MA-104 cells.
16. Incubate the MA-104 cells for 2 hr in a 37° C., 5% CO2 humidified incubator.
17. Add 5 ml of MEM supplemented with 2% FBS and 250 μg/ml Ara-C to the infected MA-104 cells.
18. Incubate the infected MA-104 cells for 3 to 4 days in a 37° C., 5% CO2 humidified incubator.
The rescued virus is now called rHPIV3-EGFP. At this point no vTF7-3 CPE should be seen. If rHPIV3-EGFP was successfully rescued, then typical HPIV-3 CPE should be seen, which is characterized by syncytia formation.
19. Remove the infected MA-104 cells from the plate with a sterile cell scraper and freeze the cell suspension in 10% glycerol at −80° C. to lyse cells. Store up to 2 years at −80° C.
Plaque-Purify Infectious rHPIV3-EGFP
20. Seed MA-104 cells in a 12-well plate at 8×105 cells/well in MEM supplemented with 10% FBS.
21. Incubate the MA-104 cells overnight in a 37° C., 5% CO2 humidified incubator.
22. Dilute the rHPIV3-EGFP virus, using serial ten-fold dilutions, by a factor of 1×10−6 in 500 μl of MEM.
23. Remove the growth medium from the MA-104 cells and add 500 μl of MEM containing each dilution of virus into individual wells.
24. Incubate the MA-104 cells for 2 hr in a 37° C., 5% CO2 humidified incubator.
25. Remove the virus/medium mixture and replace with 500 μl of the pre-warmed (>37° C.) 50:50 mixture of 2% agarose and 2×MEM.
26. Incubate the infected MA-104 cells for 2 to 3 days in a 37° C., 5% CO2 humidified incubator.
27. Select a well-isolated virus plaque located in a well in which the 10−5 or 10−6 dilution of virus was plated (these wells should have ˜1 to 20 plaques each). Remove the agarose plug directly over the isolated plaque using a 1-ml pipet and place the plug into 500 μl MEM.
28. Add 25 μl of MEM to the remaining hole from where the plug was removed to extract any remaining infectious virus. Remove the 25-μl volume of medium and add it to the 500 μl of MEM containing the agarose plug, and store at −80° C.
29. Repeat steps 20 to 28 two additional times.
30. To amplify the plaque-purified virus, remove the growth medium from newly plated MA-104 cells and add the 500 μl MEM containing one of the agarose plugs and virus.
31. Incubate the MA-104 cell mix for 2 hr in a 37° C., 5% CO2 humidified incubator.
32. Add 1.5 ml MEM supplemented with 2% FBS to the MA-104 cells.
33. Incubate the infected MA-104 cells for 3 to 5 days in a 37° C., 5% CO2 humidified incubator.
34. Scrape the infected MA-104 cells off the plate with a sterile cell scraper and freeze the cell suspension in 10% glycerol at −80° C. to lyse cells. Store up to 2 years at −80° C. The resulting virus rHPIV3-EGFP, which has been plaque-purified a total of three times, is now free of contaminating vaccinia virus.

### Reagents and Solutions

Use deionized, distilled water in all recipes and disclosure steps.

Agarose, 2% (w/v)

Bake clean glassware 2 hr at 204° C. Add 8 g of low-melting agarose (Thermo Fisher Scientific) to 400 ml of deionized, distilled water. Autoclave and store at room temperature. The agarose may be stored indefinitely as long as it is kept sterile. To reheat the stock solution, microwave on high until agarose is melted and cool to 37° C. before adding to cells.

### MEM, 2×

Dissolve one packet of powdered MEM (Invitrogen, cat. no. 61100-061) in 400 ml of deionized, distilled water. Add 30 ml of 7.5% sodium bicarbonate solution (Invitrogen) and adjust volume to 500 ml with deionized, distilled water. Sterilize by passing through a 0.2-μm filter. Store up to 2 months at 4° C.

### Parameters and Troubleshooting

### Viral DNA

To ensure that a full-length viral cDNA clone can be generated, high-quality, intact viral RNA needs to be isolated. Traditional RNA isolation, e.g., Trizol extraction, can be used but assurance that reagents are nuclease-free, e.g., DEPC-treated, is labor intensive and time consuming. Commercial kits are available that guarantee their components are nuclease-free and result in similar quantities of purified RNA. However, laboratory bench space and common laboratory equipment, e.g., pipets, which are used communally, should be decontaminated or, ideally, dedicated space and pipets should be set aside and reserved solely for RNA work. Gloves should also be worn at all times to prevent nuclease contamination from hands. In addition, new, clean, and nuclease-free pipet tips, preferably aerosol resistant, and microcentrifuge tubes should also be used at all times when working with RNA. Isolated RNA should be stored at −80° C. to prevent degradation.

### Primer Design and Synthesis

Generating a viral cDNA clone by RT-PCR amplification is also dependent on accurate primer design. The numbers of known viral genomic sequences are increasing and can be rapidly found through GenBank. Therefore, the procedure to design primers to match the genome of the desired virus with known sequence can be easily done. For example, the antigenomic sequence for the virus used in this disclosure, HPIV-3 strain 14702, is located in GenBank, accession no. EU424062. On the other hand, if the user of this disclosure is attempting to clone a virus whose genomic sequence is unknown, other procedures may be useful and several options may be considered. First, if the genomic sequences of other strains of the same virus the user is attempting to clone are known, then a consensus sequence of all known sequences can be assembled. This consensus will show conserved sequences in the viral genome that can be targeted by primers with a high degree of certainty of primer annealing. Second,

a combination of 3′ and 5′ RACE and shotgun sequencing of the viral genome can also give insight into the actual sequence of the 3′ and 5′ genomic ends and internal genomic regions, which could be used for primer design. Once the genomic 3′ end sequence is known, the primer that will anneal to this site can be designed to contain nontemplated restriction sites and the T7 promoter sequence adjacent to the first viral nucleotide separated by two guanosine residues. Once the primer sequence has been decided, the production of the primers is also important. During the synthesis of primers, the length of the primer ordered represents only a proportion of the primer actually in the tube. Therefore, the costly option to purify each primer, e.g., HPLC or PAGE, may be desired. This need to purify primers is especially crucial when synthesizing long primers, >30 nucleotides, and primers used for cloning purposes in PCR that contain additional, nontemplated nucleotides on the 5′ end of the primer, which may code for restriction sites. In addition, if the resulting PCR product is to be used directly in a ligation reaction, the presence of a phosphate on the 5′ end of the primer will facilitate the ligation of the product into the digested plasmid, especially if the plasmid is dephosphorylated or blunt ended. Finally, when reconstituting the primers used during the cDNA synthesis step, special consideration is needed. Since these primers will anneal to the viral genomic RNA strand, they need to be reconstituted in nuclease-free water or buffer and handled identically as an RNA sample would be handled.

### Lethal Mutations

A possible unforeseen and uncontrollable circumstance that may lead to the unsuccessful rescue of a recombinant virus could be the incorporation of unintentional lethal mutations in the viral genome. These mutations will most likely occur during reverse transcription of the viral genomic RNA into cDNA by the reverse transcriptase enzyme, which lacks proofreading capabilities. The size of most negative stranded viruses, ˜15 kb, increases the likelihood of one or more unintentional mutations; nonlethal with any luck. In addition, the relatively large size of the viral genomic RNA renders it highly unlikely to create a full-length viral cDNA in one strand because of the lack of processivity of the reverse transcriptase enzyme, which is inhibited by RNase H activity and RNA secondary structure. To counteract these inhibitors, reverse transcriptase enzymes should be obtained and tested that will lack RNase H activity and will be stable at temperatures up to 60° C. to eliminate secondary structure. This disclosure suggests the use of the ProStar First-Strand RT-PCR kit (Stratagene) because of the lack of RNase H activity in the reverse transcriptase enzyme. An elevated incubation temperature should be used to disrupt secondary structure. On the other hand, the advent of high fidelity DNA polymerases with proofreading capabilities has increased the likelihood that sufficient amounts of PCR products can be obtained that are true to at least the cDNA template. This disclosure suggests the use of the PfuTurbo Hotstart DNA polymerase (Stratagene) because of its high-fidelity, hotstart capabilities, and generation of blunt ends, which are needed in subsequent ligation reactions. Currently, there are additional DNA polymerases that are commercially available and possess higher fidelity rates than PfuTurbo, e.g., PfuUltra. When optimizing PCR conditions, an important aspect to consider is the primer annealing temperature. If the temperature is too low, non-specific bands appear; if too high, possibly no PCR products will be obtained. As a general rule, annealing temperatures should be 5° C. below the lowest Tm of the primer pair but can be changed in either direction. The temperatures used in this disclosure are 5° C. below the Tm for the primer pair and worked well with the GENEMate thermal cycler, but other annealing temperatures may produce better results with different thermal cyclers. Lastly, this disclosure suggests the use of the two-step approach to RT-PCR and discourages the use of the one-step approach because of the inclusion of low-fidelity DNA polymerase, compared to PfuTurbo or PfuUltra, in the super mixes.

### Cloning Controls

The cloning steps in this disclosure are probably the most time consuming and problematic because of the multiple steps. As long as the proper controls are run with each ligation reaction, troubleshooting should make the process less difficult. The main control that has proven to be the most useful during cloning is a digested plasmid that ligates to itself in the absence of an insert. A majority of the cloning steps in this disclosure involve the digestion of two restriction enzymes to allow for directional cloning. In the two-enzyme system, the self-ligated control will indicate whether the plasmid has been digested by both enzymes. Ideally, no transformants should be seen on the agar plate following transformation if both enzymes digested the plasmid properly. On the other hand, if an abundance of transformants can be seen following transformation, this indicates that one of the two enzymes did not cut the plasmid of interest and that the digestions should be repeated. A majority of the time, sequential digestions cut the DNA more efficiently than simultaneous digestions, even if the manufacturer indicates the enzymes are compatible in one buffer during a double digestion. Also, extending the length of incubation time for the second digestion also increases the number of plasmids that are digested with the enzymes and increases cloning efficiencies. In addition, as the length of the antigenomic cDNA plasmid increases, the transformation efficiency may decrease. If no transformants are seen on the agar plates and the controls indicate that both enzymes cut the plasmid, this suggests that the ligase enzyme is functional and that the bacterial cells are competent. One should then increase the volume of the transformants plated onto the agar plates until bacterial colonies are seen.

### Virus Rescue

Finally, during the rescue of the infectious, recombinant negative-stranded virus, several factors are important and noteworthy. First, the use of vTF7-3 to supply the T7 RNA polymerase and drive transcription of the genomic RNA and NP, P, and L transcripts has proven very efficient. However, vTF7-3 replicates very well in HeLa and other cell lines and can cause severe virus-induced CPE that may hinder the rescue procedure. Even though the replication of vTF7-3 can be controlled with the addition of Ara-C to the medium, the high rate of replication and resulting CPE may outweigh the benefits in some rescue systems. Alternatively, the modified vaccinia virus Ankara/T7 recombinant, MVA/T7, may be substituted for vTF7-3, which is replication-deficient in mammalian cells. However, the MVA/T7 virus is not as efficient at expressing the T7\ RNA polymerase as vTF7-3, but this deficiency may be overcome by increasing the MOI of the MVA/T7 during the rescue phase. Second, the amounts of the four plasmids used for transfection during the rescue can be varied. The amounts of each plasmid used in this disclosure were derived from a previously reported procedure outlining the rescue of an infectious, recombinant HPIV-3 strain 47885. However, other ratios of the four plasmids may also be used to successfully rescue infectious, recombinant negative-stranded viruses, such as the ratios used to rescue HPIV-3 strain JS, SeV, and MeV.

The plaques induced from the resulting rHPIV3-EGFP virus can be differentiated from wild-type HPIV-3 virus-induced plaques by visualization of green fluorescence emitted from infected cells under fluorescent microscopy (Table 3). In addition, the replication of the rHPIV3-EGFP virus can be directly quantitated after 48 hr using a fluorimeter by measuring the amount of EGFP expression in infected cells.

We claim:

1) A recombinant human parainfluenza virus cDNA clone comprising:
i) a reporter gene, and
ii) a cDNA copy of a viral antigenome of human parainfluenza virus type-3 strain 14702.

2) A recombinant human parainfluenza virus cDNA clone of claim 1, wherein said reporter gene is inserted into said cDNA copy of a viral antigenome of human parainfluenza virus type-3 strain 14702 at a position corresponding to a transcriptional unit chosen from a group consisting of transcriptional unit one, transcriptional unit two, transcriptional unit three, transcriptional unit four, transcriptional unit five, transcription unit six, and transcriptional unit seven.

3) A cDNA clone of claim 1, wherein said cDNA copy of a viral antigenome of human parainfluenza virus type-3 strain 14702 comprises a nucleotide sequence at least 95% identical to SEQ ID NO: 2.

4) A cDNA clone of claim 1, wherein said cDNA copy of a viral antigenome of human parainfluenza virus type-3 strain 14702 comprises a nucleotide sequence at least 98% identical to SEQ ID NO: 2.

5) A cDNA clone of claim 1, wherein said cDNA copy of a viral antigenome of human parainfluenza virus type-3 strain 14702 comprises a nucleotide sequence identical to SEQ ID NO: 2.

6) A cDNA clone of claim 1, wherein said reporter gene encodes an enhanced green fluorescent protein.

7) A cDNA clone of claim 1, further comprising a nucleotide sequence at least 95% identical to the sequence of SEQ ID NO: 1.

8) A cDNA clone of claim 1, further comprising a nucleotide sequence at least 98% identical to the sequence of SEQ ID NO: 1.

9) A cDNA clone of claim 1, further comprising a nucleotide sequence identical to the sequence of SEQ ID NO: 1.

10) A cDNA clone of claim 1, further comprising a nucleotide sequence at least 95% identical to SEQ ID NO: 41.

11) A cDNA clone of claim 1, further comprising a nucleotide sequence identical to SEQ ID NO: 41.

12) A cDNA clone of claim 2, wherein said human parainfluenza virus type-3 strain 14702 comprises a nucleotide sequence at least 95% identical to SEQ ID NO: 2.

13) A cDNA clone of claim 2, wherein said human parainfluenza virus type-3 strain 14702 comprises a nucleotide sequence at least 98% identical to SEQ ID NO: 2.

14) A cDNA clone of claim 2, wherein said human parainfluenza virus type-3 strain 14702 comprises a nucleotide sequence identical to SEQ ID NO: 2.

15) A cDNA clone of claim 2, wherein said reporter gene encodes an enhanced green fluorescent protein.

16) An infectious, recombinant human parainfluenza virus comprising:
i) a reporter gene, and
ii) a human parainfluenza virus type-3 strain 14702 genome.

17) An infectious, recombinant negative stranded human parainfluenza virus of claim 16, further comprising a nucleotide sequence identical to SEQ ID NO: 42.

18) A method for making a recombinant human parainfluenza virus cDNA clone comprising:
i) RT-PCR amplifying at least one human parainfluenza type-3 virus antigenomic segment, and
ii) cloning the amplified human parainfluenza type-3 virus antigenomic segment, and
iii) providing a PCR amplified reporter gene, and
iv) cloning the PCR-amplified reporter gene into at least one antigenomic cDNA segment of the RT-PCR amplified human parainfluenza type-3 virus, and
v) assembling a full-length cDNA clone.

19) The method of claim 18,
wherein the RT-PCR amplifying at least one human parainfluenza type-3 virus antigenomic segment further comprises
a) infecting cells with a human parainfluenza type-3 virus, and
b) purifying viral RNA from cells infected with a human parainfluenza type-3 virus, and
c) synthesizing at least one human parainfluenza type-3 virus antigenomic cDNA segment or segments, and
e) amplifying at least one antigenomic cDNA segment or segments of human parainfluenza type-3 virus, and
f) purifying at least one antigenomic cDNA segment or segments, and,
wherein the cloning at least one human parainfluenza virus type-3 antigenomic segment further comprises
a) purifying and ligating at least one human parainfluenza type-3 viral antigenomic cDNA segment or segments into a vector to provide for a cDNA containing vector, and
b) amplifying the cDNA containing vector, and
c) isolating the amplified cDNA containing vector, and
d) optionally screening for cDNA containing vectors, and
e) optionally sequencing cDNA containing vectors, and,
wherein the providing a PCR amplified reporter gene further comprises PCR-amplifying an EGFP open reading frame, and,
wherein the cloning the PCR-amplified reporter gene into at least one antigenomic cDNA segment of the RT-PCR amplified human parainfluenza type-3 virus antigenomic segment further comprises cloning a PCR-amplified enhanced green fluorescent protein ORF into an amplified and cloned human parainfluenza type-3 virus antigenomic cDNA segment, and,
wherein the assembling a full-length cDNA clone further comprises assembling a full-length cDNA clone at least 95% identical to SEQ ID NO: 41.

20) A method, comprising the following steps:
i) providing cells capable of being transfected with DNA plasmids and containing T7 RNA polymerase, and
ii) providing a full-length recombinant human parainfluenza virus type-3 cDNA clone at least 95% identical to SEQ ID NO: 41, and
iii) optionally providing a support plasmid containing an amplified and cloned gene encoding an amino acid sequence at least 95% identical to SEQ ID NO: 3 and encoding for a human parainfluenza virus type-3 Nucleocapsid protein, and
iv) optionally providing a support plasmid containing an amplified and cloned gene encoding an amino acid sequence at least 95% identical to SEQ ID NO: 4 and encoding for a human parainfluenza virus type-3 Phosphoprotein (SEQ ID NO: 4), and
v) optionally providing a support plasmid containing the amplified and cloned gene encoding an amino acid sequence at least 95% identical to SEQ ID NO: 9 and encoding for a human parainfluenza virus type-3 Large protein, and
vi) contacting the cells with the four DNA plasmids, and
vii) allowing sufficient time for the cells to express the human parainfluenza virus type-3 Nucleocapsid protein, the Phosphoprotein, and the Large protein, and generate sufficient genomic RNA copies of the human parainfluenza virus type-3 cDNA to allow natural virus replication cycles to occur, and
viii) recovering infectious, recombinant virus particles or virions composed of negative sense viral RNA of a human parainfluenza virus at least 95% identical to SEQ ID NO: 42 from the infected cells.

21) The method of claim 20, further comprising
i) providing cells capable of being infected with a human parainfluenza virus recovered from claim 20, and
ii) providing an antiviral compound, and
iii) providing a recombinant human parainfluenza type-3 virus that expresses an enhanced green fluorescent protein at least 95% identical to SEQ ID NO: 42, and
iv) causing the cells to be infected with the recombinant human parainfluenza type-3 virus that expresses green fluorescent protein in the presence of or with the addition of the antiviral compound, and
v) monitoring expression of the enhanced green fluorescent protein by measuring fluorescence, and
vi) optionally correlating the level of expression of green fluorescent protein with the antiviral activity of the antiviral compound.


SEQUENCE LISTING (42 sequences)

SEQ ID NO: 1 - DNA, 16314 bp, Human parainfluenza virus 3
accaaacaagagaagaaacttgtcaggaaatataaatttaacttaaaattaacttaggattaaagacattgactagaaggtcaagaacctgcaggtcgactctagaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattccaactgagcgccggttaataaataagaaaaacttaggattaaagacattgactagaaggtcaagaaaagggaactctataatttcaaaaatgttgagcctatttgatacatttaatgcacgtaggcaagaaaacataacaaaatcagctggtggagctatcattcctggacagaaaaatactgtctctatattcgcccttggaccgacaataactgatgataatgagaaaatgacattagctcttctatttctgtctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaggatctaaaacgacaaaagtatggaggatttgtggttaagacgagagagatgatatatgaaaagacaactgattggatatttggaagtgacctggattatgatcaggaaactatgttgcagaacggcagaaacaattcaacaattgaggaccttgtccacacatttgggtatccatcatgtttaggagctcttataatacagatctggatagttttagtcaaagctatcactagtatctcaggattaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagactagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgctgaagctcaaatgagctcaacactggaagatgaacttggagtgacacacgaagctaaagaaagcttgaagagacatataaggaacataaacagttcagagacatctttccacaaaccaacaggtggatcagccatagagatggcaatagatgaagagccagaacaattcgaacatagagcagatcaagaacagaatggagaacctcaatcatccataattcaatatgcctgggcagaaggaaatagaagcgatgatcagactgagcaggctacagaatctgacaatatcaagaccgaacaacaaaacatcagagacagactaaacaagagactcaatgacaagaagaaacaaagcagtcaaccacctaccaatcccacaaacagaacaaaccaggacgaaatagatgatctgtttaatgcatttggaagcaactaatcgaatcaacgttttaatccaaatcaataataaataagaaaaacttaggattaaagaatcctatcataccggaatatagagcggtaaatttagagtctgcttgcaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacctatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctagaggaatccccagaagcatcacagattctaaaaatggaacccaaaacacggaggatattgatctcaatgaaattagaaagatggataaggactctattgaggggaaaatgcgacaatctgcaaatgttccaagcgaggtatcaggaagtgatgacatacttacaacagaacaaagtagaaacagtgatcatggaagaagcctggaatctatcagtacacctgatacaagatcaataagtgttgttactgctgcaacaccagatgatgaagaagaaatactaatgaaaaacagtaggacaaagaaaagttcttcaacacatcaagaagatgacaaaagaattaaaaaagggggaaaagggaaagactggtttaagaaatcaaaagatactgacaaccagataccaacatcagactacagatccacatcaaaagggcagaagaaaatctcaaagacaacaaccatcaacaccgacacaaaggggcaaacagaaatacagacagaatcatcagaaacacaatcttcatcatggaatctcatcatcgacaacaacaccgaccgaaacgaacagacaagcacaactcctccaacaacaacttcaagatcaacctatacaaaagaatcgatccgaacaaactctgaatccaaacccaagacacaaaagacaaatggaaaggaaaggaaggatacagaagagagcaatcgatttacagagagggcaattactctattgcagaatcttggtgtaatccaatctacatcaaaattagatttatatcaagacaaacgagttgtatgtgtagcaaatgtactaaacaatgtagatactgcatcaaagatagacttcctggcaggattagtcataggggtttcaatggataacgacacaaaattaacacagatacaaaatgaaatgctaaacctcaaaacagatctaaagaaaatggacgaatcacatagaagattgatagaaaatcaaagagaacaactgtcattgatcacgtcattaatttcaaatcttaaaattatgactgagagaggaggtaagaaagaccaaaatgaatccactgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatactaagttcatataatgagtcaaatgcaacaagactaatacccaaaaaagtgagcagtacaatgagatcactagttgcagtcatcaacaacagcaacctctcacaaagcacaaaacaatcatacataaacgaactcaaacgttgcaaaaatgatgaagaagtatctgaattaatggacatgttcaatgaagatgtcaacaattgccaatgatccaacaaagaaactacactgaacaaacagacaagaaacaacagcagatcaaaatctgtcaacacacacaaaatcaagcagaataaaacaacagatatcaatcaacatacaaataagaaaaacttaggattaaagaataaattaatccttgtccaaaatgagtataactaactctgcaatatacacattcccagaatcatcattctttgaaaatggtcatatagaaccattaccactcaaagtcaatgaacagagaaaagcagtaccccacattagagttgccaaaatcggaaatccaccaaaacacggatcccggtatttagatgtcttcttacttggctttttcgagatggaacgaatcaaagacaaatacgggagtgtgaatgatctcgacagtgacccgagttacaaagtctgtggctctggatcattaccaatcggattggctaagtacactgggaatgaccaagaattgttacaagctgcaaccaaactggacatagaagtgagaagaacagttaaagcgaaagagatggttgtttacacggtacaaaatataaaaccagaactgtacccatggtccaatagactaagaaaaggaatgctgttcgatgccaacaaagttgctcttgctcctcaatgtcttccactagataggagcataaaattcagagtaatcttcgtgaattgtacagcaattggatcaataaccctgttcaaaattcccaagtcaatggcatcactatctctacccaacacaatatcaatcaatctgcaggtacacatcaaaacaggggttcagactgattctaaagggatagttcaaattttggatgagaaaggagaaaaatcactgaatttcatggtccatctcggattgattaaaagaaaagtaggcagaatgtactctgttgaatactgtaaacagaaaatcgagaaaatgagattgatattttctttaggactagttggaggaatcagtcttcatgtcaatgcaaccggatccatatcaaaaacactagcaagtcagctggtattcaagagggagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctatttcagtccagacgtatctattaagctgaagcaaataagggataatcaaaaacttaggataaaagaggtcaataccaacaaccattagcagtcatactcgcaagaataagaaaggagggatttaaaaagttaaatagaggaaatcaaaacaaaaagtacagaacaccagaacaataaaatcaaaacatccaactcactcaaaacaaaaatcccaaaagagaccagtaatacaacaagcactgagcacaatgacaacttcaatactgctaattattacaaccatgatcatggcatctttctgccaaatagatatcacaaaactacagcatgtaggtgtattggtcaacagtcccaaagggatgaagatatcacaaaactttgaaacaagatatctgattttgagcctcataccaaaaatagaagattctaactcttgtggtgaccagcagatcaagcaatacaagaagctattggatagactgatcatccctttatatgatggattaagattacagaaagatgtgatagtaaccaatcaagaatccaatgaaaacactgatcctagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgacaaatatgatatttatgatctgttatttacagaatcaataaaagtgagagttatagatgttgacttgaatgattactcaatcactctccaagtcagactccctttattaactaggctgctgaacactcagatctacaaagtagattccatatcatataacatccaaaacagagaatggtatatccctcttcccagccatattatgacgaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaatggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaattgagctcaacaaggccaaatcagatctagaagaatcaaaagaatggataagaaggtcaaatcaaaaactagattccattggaaattggcaccaatctagcactacaatcataattattttgataatgatcattatattgtttataattaatgtaacgataattacaattgcaattaagtattacagaattcaaaagagaaatcgagtggatcaaaatgacaagccgtatgtactgacaaacaaataatatatctacagatcattagatattaaaattataaaaaacttaggagtaaagttacacaatccaactctactcatataattgaggaaaaacctaatagacaaatccaaattcgagatggaatactggaagcataccaatcacggaaaggatgctggtaatgagctggagacgtccatggctactcatggcaacaagctcaccaataagataatatacatattatggacaataatcctggtgttattatcaatagtcttcatcatagtgctaaccaattccatcaaaagtgaaaagacccatgaatcattgctgcgagacataaacaatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcatcagtgaaattataattagaaatgataatcaagaagtgctgccacaaagaataacgcatgatgtaggtataaaacctttaaatccagatgatttttggagatgcacgtctggtcttccatctttaatgaaaactccaaaaataaggttaatgccagggccggggttattagctatgccaacgactgttgatggctgtattagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatactttcaacataaatgacaataggaagtcatgctctctagcactcctaaatacagatgtatatcaactgtgttcaactcccaaagttgatgaaagatcagattatgcatcatcgggcatagaagatattgtacttgatattgtcaattatgatggctcaatctcaacaacaagatttaagaataataacataagctttgatcaaccatatgctgcgctatacccatctgttggaccagggatatactacaaaggcaaaataatatttcttggatatggaggtcttgaacacccaataaatgagaatgtgatctgcaacacaactgggtgtcccgggaaaacacagagagactgtaatcaagcgtctcatagtccatggttttcagataggaggatggtcaactccatcattgttgttgacaaaggcttaaactcaactccaaaattgaaggtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccattcgaaacagaacactctcagctggatatacaacaacaagctgcattacacactataacaaaggatattgttttcatatagtagaaataaatcataaaagctcaaacacatttcaacccatgttgttcaaaacagagattccaaaaagctgcagttaatcataattaaccataatatgcattaatctatctacaacacaagtatattataagtaatcagcaatcagacaatagacaaaagggaaatataaaaaacttaggagcaaagcgtgctcagaaaatggacactgaatctaacaatggtactgtatctgacatactctatcctgagtgtcaccttaactctcctatcgttaaaggtaaaatagcacaattacacactattatgagtttacctcagccctatgatatggatgacgactcaatactagttatcactagacagaaaataaaactcaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcaaaagttcacacaacttataaatcagataaatggtataatccattcaaaacatggtttactattaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattatttccaattatgggagaaaagacatttgatgttatatcattattagaaccacttgcattatccttaattcaaactcatgatcctgttaaacaactaagaggagcttttttaaatcatgtgttatccgagatggaattaatatttgaatctagagaatcgattaaggaatttctgagtgtagattatattgataaaattttagatatatttaataaatctacaatagatgaaatagcagagattttctctttttttagaacatttgggcatcctccattagaagctagtattgcagcagaaaaggtcagaaaatatatgtatattgagaaacaattaaaatttgacactatcaataaatgtcatgctatcttctgtacaataataattaatggatatagagagagacatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaagatttgacaatttatatgaaagataaagcattatctccaaaaaaatcaaattgggatacagtttatcctgcatctaatttactgtaccgtactaacgcatccaacgaatcacgaagattagttgaagtatttatagcagatagtaaatttgatcctcatcaaatattggattatgtagaatctggggactggttagatgatccagaatttaatatttcttatagtcttaaagaaaaagagatcaaacaagaaggtagactctttgcaaaaatgacatacaaaatgagagctacacaagttttatcagagacactacttgcaaataacataggaaaattctttcaagaaaatgggatggtgaagggagagattgaattacttaagagattaacgaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttgggttaaataaattgtttaattggttacatcctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcgatacatctagcagctgttagaataggtgtgagggtgactgcaatggttcaaggagacaatcaagccatagctgtaactacaagagtacccaacaattatgactacagagttaagaaggagatagtttataaagatgtagtgagattttttgattcattaagagaagttatggatgatctaggtcatgagcttaaattaaatgaaacgattataagtagcaagatgttcatatatagcaaaagaatctattatgatgggagaattcttcctcaagctctgaaagcattatctagatgtgtcttctggtcagagacagtaatagacgaaacaagatcagcatcttcaaacttggcaacatcatttgcaaaagcaattgagaatggttattcacctgttttaggatatgcatgctcaatttttaagaatattcaacaactatatattgcccttgggatgaatattaatccaactataacacagaatatcagagatcagtattttaggaatccaaattggatgcaatatgcctctttaatacctgctagtgttgggggattcaattacatggcaatgtcaagatgttttgtaaggaatattggtgatccatcagttgccgcattagctgatattaaaagatttattaaggcgaatctattagaccgaagtgttctttataggattatgaatcaagaaccaggtgagtcatcttttttggattgggcttcagatccatattcatgcaatttaccacaatctcaaaatataaccactatgataaaaaatataacagcaaggaatgtattacaagattcaccaaatccgttattatctggattattcacaaatacaatgatagaagaagatgaagaattagctgagttcctgatggacaggaaggtaattcttcctagagttgcacatgatattctagataattctcttacaggaattagaaatgccatagctggaatgttagatacgacaaaatcactaattcgagttggcataaatagagggggactgacatatagtttgttgaggaaaatcagtaattatgatctagtacaatatgaaacactaagtaggaccttgcgattaattgtaagcgataaaatcaagtatgaagatatgtgttcagtagaccttgccatagcattgcgacaaaaaatgtggattcatttatcaggaggaaggatgataagtggacttgaaacgcctgacccattagaactactatctggggtagtaataacaggatcagaacattgtaaaatatgttattcttcagatggaacaaacccatatacttggatgtatttacccggtaatatcaaaataggatcagcagaaacaggtatatcatcattaagagttccttactttggatcagtcactgatgaaagatctgaggcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttgacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagataccaatcttatttatcaacaaataatgttaacaggattaagtgttttcgaatatttatttagattaaaagaaaccacaggacataaccctatagttatgcatctgcacatagaagatgagtgttgtattaaagaaagttttaatgatgaacatattaatccagaatctacattagaattaattcgatatcctgaaagtaatgaatttatttatgataaagacccactcaaagatgtggacttatcaaaacttatggttattaaagaccattcttacacaattgatatgaattattgggatgataccgatatcatacatgcaatttcaatatgtactgcaattacaatagcagatactatgtcacaattagatcgagataatttaaaagagataatagtcattgcaaatgatgatgatattaatagcttaatcactgaatttttgactcttgacatacttgtatttcttaagacatttggtggattattagtaaatcaatttgcatacactctttatagtttaaaaatagaaggtagggatctcatttgggattatataatgagaacactgagagatacttcccattcaatattaaaagtattatctaatgcattatctcatcctaaggtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatgtacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgtggtattagtccacctgaagtaattgatgattgggatccggtagaagatgaaaatatgctggataacattgtcaaaactataaatgataactgcaataaagataataaagggaataaaattaacaatttctggggactagcactcaagaactatcaagtccttaaaatcagatctataacaagtgattctgatgataatgatagactagatgctaatacaagtggtttgacacttccccaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggcaaaaaattaggaaatgtgacacagattcttaacagggtaaaagtactgttcaatgggaatcccaattcaacatggataggaaatatggaatgtgagagcttgatatggagtgaattaaatgataagtctattggattagtacattgtgatatggaaggagccatcggcaaatcagaagaaactgttctacatgaacattatagtgttataagaattacatacttgattggggatgatgatgttgttttagtttccaagattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaagaataatgaatggttacatcatgaaatcaaagaaggagaaagagattatggagtcatgagaccatatcatatggcactacaaatctttggatttcaaatcaatttaaatcatctagcgaaagaatttttatcgaccccagatctgactaatatcaacaatataatccaaagttttcagcggacaatcaaggatgttttatttgaatggatcaatataactcatgatgataagagacacaaattaggcgggagatataacatattcccactgaaaaataagggaaagttaagactgctatcaagaagactagtattaagttggatttcattatcattatcgactcgattacttacaggtcgctttcctgatgaaaaatttgaacatagagcacagacaggatatgtatcattagctgatactgatttagaatcattaaagttattgtcgaaaaacgtcattaagaattacagagagtgtataggatcaatatcatattggtttctaactaaggaagttaaaatacttatgaaattgattggtggtgctaaattattaggaattcccagacaatataaagaacccgaagatcagttattagaaaactacaatcaatatgatgaatttgatatcgattaaaacataaatacaatgaagatatatcctaacctttatctttaagcctaaggatagacaaaaagtaagaaaaacatgtaatatatatataccacacagagtgcttctcttgtttggt

SEQ ID NO: 2 - DNA, 27432 bp, Human parainfluenza virus 3
accaaacaagagaagaaacttgtcaggaaatataaatttaacttaaaattaacttaggattaaagacattgactagaaggtcaagaaaagggaactctataatttcaaaaatgttgagcctatttgatacatttaatgcacgtaggcaagaaaacataacaaaatcagctggtggagctatcattcctggacagaaaaatactgtctctatattcgcccttggaccgacaataactgatgataatgagaaaatgacattagctcttctatttctgtctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaggatctaaaacgacaaaagtatggaggatttgtggttaagacgagagagatgatatatgaaaagacaactgattggatatttggaagtgacctggattatgatcaggaaactatgttgcagaacggcagaaacaattcaacaattgaggaccttgtccacacatttgggtatccatcatgtttaggagctcttataatacagatctggatagttttagtcaaagctatcactagtatctcaggattaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagactagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgctgaagctcaaatgagctcaacactggaagatgaacttggagtgacacacgaagctaaagaaagcttgaagagacatataaggaacataaacagttcagagacatctttccacaaaccaacaggtggatcagccatagagatggcaatagatgaagagccagaacaattcgaacatagagcagatcaagaacagaatggagaacctcaatcatccataattcaatatgcctgggcagaaggaaatagaagcgatgatcagactgagcaggctacagaatctgacaatatcaagaccgaacaacaaaacatcagagacagactaaacaagagactcaatgacaagaagaaacaaagcagtcaaccacctaccaatcccacaaacagaacaaaccaggacgaaatagatgatctgtttaatgcatttggaagcaactaatcgaatcaacgttttaatccaaatcaataataaataagaaaaacttaggattaaagaatcctatcataccggaatatagagcggtaaatttagagtctgcttgcaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacctatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctagaggaatccccagaagcatcacagattctaaaaatggaacccaaaacacggaggatattgatctcaatgaaattagaaagatggataaggactctattgaggggaaaatgcgacaatctgcaaatgttccaagcgaggtatcaggaagtgatgacatacttacaacagaacaaagtagaaacagtgatcatggaagaagcctggaatctatcagtacacctgatacaagatcaataagtgttgttactgctgcaacaccagatgatgaagaagaaatactaatgaaaaacagtaggacaaagaaaagttcttcaacacatcaagaagatgacaaaagaattaaaaaagggggaaaagggaaagactggtttaagaaatcaaaagatactgacaaccagataccaacatcagactacagatccacatcaaaagggcagaagaaaatctcaaagacaacaaccatcaacaccgacacaaaggggcaaacagaaatacagacagaatcatcagaaacacaatcttcatcatggaatctcatcatcgacaacaacaccgaccgaaacgaacagacaagcacaactcctccaacaacaacttcaagatcaacctatacaaaagaatcgatccgaacaaactctgaatccaaacccaagacacaaaagacaaatggaaaggaaaggaaggatacagaagagagcaatcgatttacagagagggcaattactctattgcagaatcttggtgtaatccaatctacatcaaaattagatttatatcaagacaaacgagttgtatgtgtagcaaatgtactaaacaatgtagatactgcatcaaagatagacttcctggcaggattagtcataggggtttcaatggataacgacacaaaattaacacagatacaaaatgaaatgctaaacctcaaaacagatctaaagaaaatggacgaatcacatagaagattgatagaaaatcaaagagaacaactgtcattgatcacgtcattaatttcaaatcttaaaattatgactgagagaggaggtaagaaagaccaaaatgaatccactgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatactaagttcatataatgagtcaaatgcaacaagactaatacccaaaaaagtgagcagtacaatgagatcactagttgcagtcatcaacaacagcaacctctcacaaagcacaaaacaatcatacataaacgaactcaaacgttgcaaaaatgatgaagaagtatctgaattaatggacatgttcaatgaagatgtcaacaattgccaatgatccaacaaagaaactacactgaacaaacagacaagaaacaacagcagatcaaaatctgtcaacacacacaaaatcaagcagaataaaacaacagatatcaatcaacatacaaataagaaaaacttaggattaaagaataaattaatccttgtccaaaatgagtataactaactctgcaatatacacattcccagaatcatcattctttgaaaatggtcatatagaaccattaccactcaaagtcaatgaacagagaaaagcagtaccccacattagagttgccaaaatcggaaatccaccaaaacacggatcccggtatttagatgtcttcttacttggctttttcgagatggaacgaatcaaagacaaatacgggagtgtgaatgatctcgacagtgacccgagttacaaagtctgtggctctggatcattaccaatcggattggctaagtacactgggaatgaccaagaattgttacaagctgcaaccaaactggacatagaagtgagaagaacagttaaagcgaaagagatggttgtttacacggtacaaaatataaaaccagaactgtacccatggtccaatagactaagaaaaggaatgctgttcgatgccaacaaagttgctcttgctcctcaatgtcttccactagataggagcataaaattcagagtaatcttcgtgaattgtacagcaattggatcaataaccctgttcaaaattcccaagtcaatggcatcactatctctacccaacacaatatcaatcaatctgcaggtacacatcaaaacaggggttcagactgattctaaagggatagttcaaattttggatgagaaaggagaaaaatcactgaatttcatggtccatctcggattgattaaaagaaaagtaggcagaatgtactctgttgaatactgtaaacagaaaatcgagaaaatgagattgatattttctttaggactagttggaggaatcagtcttcatgtcaatgcaaccggatccatatcaaaaacactagcaagtcagctggtattcaagagggagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctatttcagtccagacgtatctattaagctgaagcaaataagggataatcaaaaacttaggataaaagaggtcaataccaacaaccattagcagtcatactcgcaagaataagaaaggagggatttaaaaagttaaatagaggaaatcaaaacaaaaagtacagaacaccagaacaataaaatcaaaacatccaactcactcaaaacaaaaatcccaaaagagaccagtaatacaacaagcactgagcacaatgacaacttcaatactgctaattattacaaccatgatcatggcatctttctgccaaatagatatcacaaaactacagcatgtaggtgtattggtcaacagtcccaaagggatgaagatatcacaaaactttgaaacaagatatctgattttgagcctcataccaaaaatagaagattctaactcttgtggtgaccagcagatcaagcaatacaagaagctattggatagactgatcatccctttatatgatggattaagattacagaaagatgtgatagtaaccaatcaagaatccaatgaaaacactgatcctagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgacaaatatgatatttatgatctgttatttacagaatcaataaaagtgagagttatagatgttgacttgaatgattactcaatcactctccaagtcagactccctttattaactaggctgctgaacactcagatctacaaagtagattccatatcatataacatccaaaacagagaatggtatatccctcttcccagccatattatgacgaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaatggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaattgagctcaacaaggccaaatcagatctagaagaatcaaaagaatggataagaaggtcaaatcaaaaactagattccattggaaattggcaccaatctagcactacaatcataattattttgataatgatcattatattgtttataattaatgtaacgataattacaattgcaattaagtattacagaattcaaaagagaaatcgagtggatcaaaatgacaagccgtatgtactgacaaacaaataatatatctacagatcattagatattaaaattataaaaaacttaggagtaaagttacacaatccaactctactcatataattgaggaaaaacctaatagacaaatccaaattcgagatggaatactggaagcataccaatcacggaaaggatgctggtaatgagctggagacgtccatggctactcatggcaacaagctcaccaataagataatatacatattatggacaataatcctggtgttattatcaatagtcttcatcatagtgctaaccaattccatcaaaagtgaaaagacccatgaatcattgctgcgagacataaacaatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcatcagtgaaattataattagaaatgataatcaagaagtgctgccacaaagaataacgcatgatgtaggtataaaacctttaaatccagatgatttttggagatgcacgtctggtcttccatctttaatgaaaactccaaaaataaggttaatgccagggccggggttattagctatgccaacgactgttgatggctgtattagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatactttcaacataaatgacaataggaagtcatgctctctagcactcctaaatacagatgtatatcaactgtgttcaactcccaaagttgatgaaagatcagattatgcatcatcgggcatagaagatattgtacttgatattgtcaattatgatggctcaatctcaacaacaagatttaagaataataacataagctttgatcaaccatatgctgcgctatacccatctgttggaccagggatatactacaaaggcaaaataatatttcttggatatggaggtcttgaacacccaataaatgagaatgtgatctgcaacacaactgggtgtcccgggaaaacacagagagactgtaatcaagcgtctcatagtccatggttttcagataggaggatggtcaactccatcattgttgttgacaaaggcttaaactcaactccaaaattgaaggtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccattcgaaacagaacactctcagctggatatacaacaacaagctgcattacacactataacaaaggatattgttttcatatagtagaaataaatcataaaagctcaaacacatttcaacccatgttgttcaaaacagagattccaaaaagctgcagttaatcataattaaccataatatgcattaatctatctacaacacaagtatattataagtaatcagcaatcagacaatagacaaaagggaaatataaaaaacttaggagcaaagcatgctcagaaaatggacactgaatctaacaatggtactgtatctgacatactctatcctgagtgtcaccttaactctcctatcgttaaaggtaaaatagcacaattacacactattatgagtttacctcagccctatgatatggatgacgactcaatactagttatcactagacagaaaataaaactcaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcaaaagttcacacaacttataaatcagataaatggtataatccattcaaaacatggtttactattaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattatttccaattatgggagaaaagacatttgatgttatatcattattagaaccacttgcattatccttaattcaaactcatgatcctgttaaacaactaagaggagcttttttaaatcatgtgttatccgagatggaattaatatttgaatctagagaatcgattaaggaatttctgagtgtagattatattgataaaattttagatatatttaataaatctacaatagatgaaatagcagagattttctctttttttagaacatttgggcatcctccattagaagctagtattgcagcagaaaaggtcagaaaatatatgtatattgagaaacaattaaaatttgacactatcaataaatgtcatgctatcttctgtacaataataattaatggatatagagagagacatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaagatttgacaatttatatgaaagataaagcattatctccaaaaaaatcaaattgggatacagtttatcctgcatctaatttactgtaccgtactaacgcatccaacgaatcacgaagattagttgaagtatttatagcagatagtaaatttgatcctcatcaaatattggattatgtagaatctggggactggttagatgatccagaatttaatatttcttatagtcttaaagaaaaagagatcaaacaagaaggtagactctttgcaaaaatgacatacaaaatgagagctacacaagttttatcagagacactacttgcaaataacataggaaaattctttcaagaaaatgggatggtgaagggagagattgaattacttaagagattaacgaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttgggttaaataaattgtttaattggttacatcctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcgatacatctagcagctgttagaataggtgtgagggtgactgcaatggttcaaggagacaatcaagccatagctgtaactacaagagtacccaacaattatgactacagagttaagaaggagatagtttataaagatgtagtgagattttttgattcattaagagaagttatggatgatctaggtcatgagcttaaattaaatgaaacgattataagtagcaagatgttcatatatagcaaaagaatctattatgatgggagaattcttcctcaagctctgaaagcattatctagatgtgtcttctggtcagagacagtaatagacgaaacaagatcagcatcttcaaacttggcaacatcatttgcaaaagcaattgagaatggttattcacctgttttaggatatgcatgctcaatttttaagaatattcaacaactatatattgcccttgggatgaatattaatccaactataacacagaatatcagagatcagtattttaggaatccaaattggatgcaatatgcctctttaatacctgctagtgttgggggattcaattacatggcaatgtcaagatgttttgtaaggaatattggtgatccatcagttgccgcattagctgatattaaaagatttattaaggcgaatctattagaccgaagtgttctttataggattatgaatcaagaaccaggtgagtcatcttttttggattgggcttcagatccatattcatgcaatttaccacaatctcaaaatataaccactatgataaaaaatataacagcaaggaatgtattacaagattcaccaaatccgttattatctggattattcacaaatacaatgatagaagaagatgaagaattagctgagttcctgatggacaggaaggtaattcttcctagagttgcacatgatattctagataattctcttacaggaattagaaatgccatagctggaatgttagatacgacaaaatcactaattcgagttggcataaatagagggggactgacatatagtttgttgaggaaaatcagtaattatgatctagaccaaacaagagaagaaacttgtcaggaaatataaatttaacttaaaattaacttaggattaaagacattgactagaaggtcaagaaaagggaactctataatttcaaaaatgttgagcctatttgatacatttaatgcacgtaggcaagaaaacataacaaaatcagctggtggagctatcattcctggacagaaaaatactgtctctatattcgcccttggaccgacaataactgatgataatgagaaaatgacattagctcttctatttctgtctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaggatctaaaacgacaaaagtatggaggatttgtggttaagacgagagagatgatatatgaaaagacaactgattggatatttggaagtgacctggattatgatcaggaaactatgttgcagaacggcagaaacaattcaacaattgaggaccttgtccacacatttgggtatccatcatgtttaggagctcttataatacagatctggatagttttagtcaaagctatcactagtatctcaggattaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagactagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgctgaagctcaaatgagctcaacactggaagatgaacttggagtgacacacgaagctaaagaaagcttgaagagacatataaggaacataaacagttcagagacatctttccacaaaccaacaggtggatcagccatagagatggcaatagatgaagagccagaacaattcgaacatagagcagatcaagaacagaatggagaacctcaatcatccataattcaatatgcctgggcagaaggaaatagaagcgatgatcagactgagcaggctacagaatctgacaatatcaagaccgaacaacaaaacatcagagacagactaaacaagagactcaatgacaagaagaaacaaagcagtcaaccacctaccaatcccacaaacagaacaaaccaggacgaaatagatgatctgtttaatgcatttggaagcaactaatcgaatcaacgttttaatccaaatcaataataaataagaaaaacttaggattaaagaatcctatcataccggaatatagagcggtaaatttagagtctgcttgcaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacctatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctagaggaatccccagaagcatcacagattctaaaaatggaacccaaaacacggaggatattgatctcaatgaaattagaaagatggataaggactctattgaggggaaaatgcgacaatctgcaaatgttccaagcgaggtatcaggaagtgatgacatacttacaacagaacaaagtagaaacagtgatcatggaagaagcctggaatctatcagtacacctgatacaagatcaataagtgttgttactgctgcaacaccagatgatgaagaagaaatactaatgaaaaacagtaggacaaagaaaagttcttcaacacatcaagaagatgacaaaagaattaaaaaagggggaaaagggaaagactggtttaagaaatcaaaagatactgacaaccagataccaacatcagactacagatccacatcaaaagggcagaagaaaatctcaaagacaacaaccatcaacaccgacacaaaggggcaaacagaaatacagacagaatcatcagaaacacaatcttcatcatggaatctcatcatcgacaacaacaccgaccgaaacgaacagacaagcacaactcctccaacaacaacttcaagatcaacctatacaaaagaatcgatccgaacaaactctgaatccaaacccaagacacaaaagacaaatggaaaggaaaggaaggatacagaagagagcaatcgatttacagagagggcaattactctattgcagaatcttggtgtaatccaatctacatcaaaattagatttatatcaagacaaacgagttgtatgtgtagcaaatgtactaaacaatgtagatactgcatcaaagatagacttcctggcaggattagtcataggggtttcaatggataacgacacaaaattaacacagatacaaaatgaaatgctaaacctcaaaacagatctaaagaaaatggacgaatcacatagaagattgatagaaaatcaaagagaacaactgtcattgatcacgtcattaatttcaaatcttaaaattatgactgagagaggaggtaagaaagaccaaaatgaatccactgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatactaagttcatataatgagtcaaatgcaacaagactaatacccaaaaaagtgagcagtacaatgagatcactagttgcagtcatcaacaacagcaacctctcacaaagcacaaaacaatcatacataaacgaactcaaacgttgcaaaaatgatgaagaagtatctgaattaatggacatgttcaatgaagatgtcaacaattgccaatgatccaacaaagaaactacactgaacaaacagacaagaaacaacagcagatcaaaatctgtcaacacacacaaaatcaagcagaataaaacaacagatatcaatcaacatacaaataagaaaaacttaggattaaagaataaattaatccttgtccaaaatgagtataactaactctgcaatatacacattcccagaatcatcattctttgaaaatggtcatatagaaccattaccactcaaagtcaatgaacagagaaaagcagtaccccacattagagttgccaaaatcggaaatccaccaaaacacggatcccggtatttagatgtcttcttacttggctttttcgagatggaacgaatcaaagacaaatacgggagtgtgaatgatctcgacagtgacccgagttacaaagtctgtggctctggatcattaccaatcggattggctaagtacactgggaatgaccaagaattgttacaagctgcaaccaaactggacatagaagtgagaagaacagttaaagcgaaagagatggttgtttacacggtacaaaatataaaaccagaactgtacccatggtccaatagactaagaaaaggaatgctgttcgatgccaacaaagttgctcttgctcctcaatgtcttccactagataggagcataaaattcagagtaatcttcgtgaattgtacagcaattggatcaataaccctgttcaaaattcccaagtcaatggcatcactatctctacccaacacaatatcaatcaatctgcaggtacacatcaaaacaggggttcagactgattctaaagggatagttcaaattttggatgagaaaggagaaaaatcactgaatttcatggtccatctcggattgattaaaagaaaagtaggcagaatgtactctgttgaatactgtaaacagaaaatcgagaaaatgagattgatattttctttaggactagttggaggaatcagtcttcatgtcaatgcaaccggatccatatcaaaaacactagcaagtcagctggtattcaagagggagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctatttcagtccagacgtatctattaagctgaagcaaataagggataatcaaaaacttaggataaaagaggtcaataccaacaaccattagcagtcatactcgcaagaataagaaaggagggatttaaaaagttaaatagaggaaatcaaaacaaaaagtacagaacaccagaacaataaaatcaaaacatccaactcactcaaaacaaaaatcccaaaagagaccagtaatacaacaagcactgagcacaatgacaacttcaatactgctaattattacaaccatgatcatggcatctttctgccaaatagatatcacaaaactacagcatgtaggtgtattggtcaacagtcccaaagggatgaagatatcacaaaactttgaaacaagatatctgattttgagcctcataccaaaaatagaagattctaactcttgtggtgaccagcagatcaagcaatacaagaagctattggatagactgatcatccctttatatgatggattaagattacagaaagatgtgatagtaaccaatcaagaatccaatgaaaacactgatcctagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgacaaatatgatatttatgatctgttatttacagaatcaataaaagtgagagttatagatgttgacttgaatgattactcaatcactctccaagtcagactccctttattaactaggctgctgaacactcagatctacaaagtagattccatatcatataacatccaaaacagagaatggtatatccctcttcccagccatattatgacgaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaatggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaattgagctcaacaaggccaaatcagatctagaagaatcaaaagaatggataagaaggtcaaatcaaaaactagattccattggaaattggcaccaatctagcactacaatcataattattttgataatgatcattatattgtttataattaatgtaacgataattacaattgcaattaagtattacagaattcaaaagagaaatcgagtggatcaaaatgacaagccgtatgtactgacaaacaaataatatatctacagatcattagatattaaaattataaaaaacttaggagtaaagttacacaatccaactctactcatataattgaggaaaaacctaatagacaaatccaaattcgagatggaatactggaagcataccaatcacggaaaggatgctggtaatgagctggagacgtccatggctactcatggcaacaagctcaccaataagataatatacatattatggacaataatcctggtgttattatcaatagtcttcatcatagtgctaaccaattccatcaaaagtgaaaagacccatgaatcattgctgcgagacataaacaatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcatcagtgaaattataattagaaatgataatcaagaagtgctgccacaaagaataacgcatgatgtaggtataaaacctttaaatccagatgatttttggagatgcacgtctggtcttccatctttaatgaaaactccaaaaataaggttaatgccagggccggggttattagctatgccaacgactgttgatggctgtattagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatactttcaacataaatgacaataggaagtcatgctctctagcactcctaaatacagatgtatatcaactgtgttcaactcccaaagttgatgaaagatcagattatgcatcatcgggcatagaagatattgtacttgatattgtcaattatgatggctcaatctcaacaacaagatttaagaataataacataagctttgatcaaccatatgctgcgctatacccatctgttggaccagggatatactacaaaggcaaaataatatttcttggatatggaggtcttgaacacccaataaatgagaatgtgatctgcaacacaactgggtgtcccgggaaaacacagagagactgtaatcaagcgtctcatagtccatggttttcagataggaggatggtcaactccatcattgttgttgacaaaggcttaaactcaactccaaaattgaaggtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccattcgaaacagaacactctcagctggatatacaacaacaagctgcattacacactataacaaaggatattgttttcatatagtagaaataaatcataaaagctcaaacacatttcaacccatgttgttcaaaacagagattccaaaaagctgcagttaatcataattaaccataatatgcattaatctatctacaacacaagtatattataagtaatcagcaatcagacaatagacaaaagggaaatataaaaaacttaggagcaaagcatgctcagaaaatggacactgaatctaacaatggtactgtatctgacatactctatcctgagtgtcaccttaactctcctatcgttaaaggtaaaatagcacaattacacactattatgagtttacctcagccctatgatatggatgacgactcaatactagttatcactagacagaaaataaaactcaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcaaaagttcacacaacttataaatcagataaatggtataatccattcaaaacatggtttactattaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattatttccaattatgggagaaaagacatttgatgttatatcattattagaaccacttgcattatccttaattcaaactcatgatcctgttaaacaactaagaggagcttttttaaatcatgtgttatccgagatggaattaatatttgaatctagagaatcgattaaggaatttctgagtgtagattatattgataaaattttagatatatttaataaatctacaatagatgaaatagcagagattttctctttttttagaacatttgggcatcctccattagaagctagtattgcagcagaaaaggtcagaaaatatatgtatattgagaaacaattaaaatttgacactatcaataaatgtcatgctatcttctgtacaataataattaatggatatagagagagacatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaagatttgacaatttatatgaaagataaagcattatctccaaaaaaatcaaattgggatacagtttatcctgcatctaatttactgtaccgtactaacgcatccaacgaatcacgaagattagttgaagtatttatagcagatagtaaatttgatcctcatcaaatattggattatgtagaatctggggactggttagatgatccagaatttaatatttcttatagtcttaaagaaaaagagatcaaacaagaaggtagactctttgcaaaaatgacatacaaaatgagagctacacaagttttatcagagacactacttgcaaataacataggaaaattctttcaagaaaatgggatggtgaagggagagattgaattacttaagagattaacgaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttgggttaaataaattgtttaattggttacatcctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcgatacatctagcagctgttagaataggtgtgagggtgactgcaatggttcaaggagacaatcaagccatagctgtaactacaagagtacccaacaattatgactacagagttaagaaggagatagtttataaagatgtagtgagattttttgattcattaagagaagttatggatgatctaggtcatgagcttaaattaaatgaaacgattataagtagcaagatgttcatatatagcaaaagaatctattatgatgggagaattcttcctcaagctctgaaagcattatctagatgtgtcttctggtcagagacagtaatagacgaaacaagatcagcatcttcaaacttggcaacatcatttgcaaaagcaattgagaatggttattcacctgttttaggatatgcatgctcaatttttaagaatattcaacaactatatattgcccttgggatgaatattaatccaactataacacagaatatcagagatcagtattttaggaatccaaattggatgcaatatgcctctttaatacctgctagtgttgggggattcaattacatggcaatgtcaagatgttttgtaaggaatattggtgatccatcagttgccgcattagctgatattaaaagatttattaaggcgaatctattagaccgaagtgttctttataggattatgaatcaagaaccaggtgagtcatcttttttggattgggcttcagatccatattcatgcaatttaccacaatctcaaaatataaccactatgataaaaaatataacagcaaggaatgtattacaagattcaccaaatccgttattatctggattattcacaaatacaatgatagaagaagatgaagaattagctgagttcctgatggacaggaaggtaattcttcctagagttgcacatgatattctagataattctcttacaggaattagaaatgccatagctggaatgttagatacgacaaaatcactaattcgagttggcataaatagagggggactgacatatagtttgttgaggaaaatcagtaattatgatctagtacaatatgaaacactaagtaggaccttgcgattaattgtaagcgataaaatcaagtatgaagatatgtgttcagtagaccttgccatagcattgcgacaaaaaatgtggattcatttatcaggaggaaggatgataagtggacttgaaacgcctgacccattagaactactatctggggtagtaataacaggatcagaacattgtaaaatatgttattcttcagatggaacaaacccatatacttggatgtatttacccggtaatatcaaaataggatcagcagaaacaggtatatcatcattaagagttccttactttggatcagtcactgatgaaagatctgaggcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttgacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagataccaatcttatttatcaacaaataatgttaacaggattaagtgttttcgaatatttatttagattaaaagaaaccacaggacataaccctatagttatgcatctgcacatagaagatgagtgttgtattaaagaaagttttaatgatgaacatattaatccagaatctacattagaattaattcgatatcctgaaagtaatgaatttatttatgataaagacccactcaaagatgtggacttatcaaaacttatggttattaaagaccattcttacacaattgatatgaattattgggatgataccgatatcatacatgcaatttcaatatgtactgcaattacaatagcagatactatgtcacaattagatcgagataatttaaaagagataatagtcattgcaaatgatgatgatattaatagcttaatcactgaatttttgactcttgacatacttgtatttcttaagacatttggtggattattagtaaatcaatttgcatacactctttatagtttaaaaatagaaggtagggatctcatttgggattatataatgagaacactgagagatacttcccattcaatattaaaagtattatctaatgcattatctcatcctaaggtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatgtacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgtggtattagtccacctgaagtaattgatgattgggatccggtagaagatgaaaatatgctggataacattgtcaaaactataaatgataactgcaataaagataataaagggaataaaattaacaatttctggggactagcactcaagaactatcaagtccttaaaatcagatctataacaagtgattctgatgataatgatagactagatgctaatacaagtggtttgacacttccccaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggcaaaaaattaggaaatgtgacacagattcttaacagggtaaaagtactgttcaatgggaatcccaattcaacatggataggaaatatggaatgtgagagcttgatatggagtgaattaaatgataagtctattggattagtacattgtgatatggaaggagccatcggcaaatcagaagaaactgttctacatgaacattatagtgttataagaattacatacttgattggggatgatgatgttgttttagtttccaagattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaagaataatgaatggttacatcatgaaatcaaagaaggagaaagagattatggagtcatgagaccatatcatatggcactacaaatctttggatttcaaatcaatttaaatcatctagcgaaagaatttttatcgaccccagatctgactaatatcaacaatataatccaaagttttcagcggacaatcaaggatgttttatttgaatggatcaatataactcatgatgataagagacacaaattaggcgggagatataacatattcccactgaaaaataagggaaagttaagactgctatcaagaagactagtattaagttggatttcattatcattatcgactcgattacttacaggtcgctttcctgatgaaaaatttgaacatagagcacagacaggatatgtatcattagctgatactgatttagaatcattaaagttattgtcgaaaaacgtcattaagaattacagagagtgtataggatcaatatcatattggtttctaactaaggaagttaaaatacttatgaaattgattggtggtgctaaattattaggaattcccagacaatataaagaacccgaagatcagttattagaaaactacaatcaatatgatgaatttgatatcgattaaaacataaatacaatgaagatatatcctaacctttatctttaagcctaaggatagacaaaaagtaagaaaaacatgtaatatatatataccaaacagagttcttctcttgtttggt

SEQ ID NO: 3 - protein, 515 aa, Human parainfluenza virus 3
MLSLFDTFNARRQENITKSAGGAIIPGQKNTVSIFALGPTITDDNEKMTLALLFLSHSLDNEKQHAQRAGFLVSLLSMAYANPELYLTTNGSNADVKYVIYMIEKDLKRQKYGGFVVKTREMIYEKTTDWIFGSDLDYDQETMLQNGRNNSTIEDLVHTFGYPSCLGALIIQIWIVLVKAITSISGLRKGFFTRLEAFRQDGTVQAGLVLSGDTVDQIGSIMRSQQSLVTLMVETLITMNTSRNDLTTIEKNIQIVGNYIRDAGLASFFNTIRYGIETRMAALTLSTLRPDINRLKALMELYLSKGPRAPFICILRDPIHGEFAPGNYPAIWSYAMGVAVVQNRAMQQYVTGRSYLDIDMFQLGQAVARDAEAQMSSTLEDELGVTHEAKESLKRHIRNINSSETSFHKPTGGSAIEMAIDEEPEQFEHRADQEQNGEPQSSIIQYAWAEGNRSDDQTEQATESDNIKTEQQNIRDRLNKRLNDKKKQSSQPPTNPTNRTNQDEIDDLFNAFGSN

SEQ ID NO: 4 - protein, 603 aa, Human parainfluenza virus 3
MESDAKNYQIMDSWEEESRDKSTNISSALNIIEFILSTDPQEDLSENDTINTRTQQLSATICQPEIKPTETSEKDSGSTDKNRQSGSSHECTTEAKDRNIDQETVQRGPGRRSSSDSRAETVVSRGIPRSITDSKNGTQNTEDIDLNEIRKMDKDSIEGKMRQSANVPSEVSGSDDILTTEQSRNSDHGRSLESISTPDTRSISVVTAATPDDEEEILMKNSRTKKSSSTHQEDDKRIKKGGKGKDWFKKSKDTDNQIPTSDYRSTSKGQKKISKTTTINTDTKGQTEIQTESSETQSSSWNLIIDNNTDRNEQTSTTPPTTTSRSTYTKESIRTNSESKPKTQKTNGKERKDTEESNRFTERAITLLQNLGVIQSTSKLDLYQDKRVVCVANVLNNVDTASKIDFLAGLVIGVSMDNDTKLTQIQNEMLNLKTDLKKMDESHRRLIENQREQLSLITSLISNLKIMTERGGKKDQNESTERVSMIKTKLKEEKIKKTRFDPLMEAQGIDKNIPDLYRHAGDTLENDVQVKSEILSSYNESNATRLIPKKVSSTMRSLVAVINNSNLSQSTKQSYINELKRCKNDEEVSELMDMFNEDVNNCQ

SEQ ID NO: 5 - protein, 199 aa, Human parainfluenza virus 3
MLKTIKSWILGKRNQEINQLISPRPSTSLNSYSAPTPKKTYRKTTQSTQEPSNSVPPSVNQKSNQQKQVRKIVDQLTKIDSLGHHTNVQQKQKIEILIRKLYREDLGEEAAQIVELRLWSLEESPEASQILKMEPKTRRILISMKLERWIRTLLRGKCDNLQMFQARYQEVMTYLQQNKVETVIMEEAWNLSVHLIQDQ

SEQ ID NO: 6 - protein, 353 aa, Human parainfluenza virus 3
MSITNSAIYTFPESSFFENGHIEPLPLKVNEQRKAVPHIRVAKIGNPPKHGSRYLDVFLLGFFEMERIKDKYGSVNDLDSDPSYKVCGSGSLPIGLAKYTGNDQELLQAATKLDIEVRRTVKAKEMVVYTVQNIKPELYPWSNRLRKGMLFDANKVALAPQCLPLDRSIKFRVIFVNCTAIGSITLFKIPKSMASLSLPNTISINLQVHIKTGVQTDSKGIVQILDEKGEKSLNFMVHLGLIKRKVGRMYSVEYCKQKIEKMRLIFSLGLVGGISLHVNATGSISKTLASQLVFKREICYPLMDLNPHLNLVIWASSVEITRVDAIFQPSLPGEFRYYPNIIAKGVGKIKQWN

SEQ ID NO: 7 - protein, 539 aa, Human parainfluenza virus 3
MTTSILLIITTMIMASFCQIDITKLQHVGVLVNSPKGMKISQNFETRYLILSLIPKIEDSNSCGDQQIKQYKKLLDRLIIPLYDGLRLQKDVIVTNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEIESCLSGNISQCPRTTVTSDIVPRYAFVNGGVVANCITTTCTCNGIGNRINQPPDQGIKIITHKECSTIGINGMLFNTNKEGTLAFYTPNDITLNNSVALDPIDISIELNKAKSDLEESKEWIRRSNQKLDSIGNWHQSSTTIIIILIMIIILFIINVTIITIAIKYYRIQKRNRVDQNDKPYVLTNK

SEQ ID NO: 8 - protein, 572 aa, Human parainfluenza virus 3
MEYWKHTNHGKDAGNELETSMATHGNKLTNKIIYILWTIILVLLSIVFIIVLTNSIKSEKTHESLLRDINNEFMEITEKIQMASDNTNDLIQSGVNTRLLTIQSHVQNYIPISLTQQMSDLRKFISEIIIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSTPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNRTLSAGYTTTSCITHYNKGYCFHIVEINHKSSNTFQPMLFKTEIPKSCS

SEQ ID NO: 9 - protein, 2233 aa, Human parainfluenza virus 3
MDTESNNGTVSDILYPECHLNSPIVKGKIAQLHTIMSLPQPYDMDDDSILVITRQKIKLNKLDKRQRSIRRLKLILTEKVNDLGKYTFIRYPEMSKEMFKLYIPGINSKVTELLLKADRTYSQMTDGLRDLWINVLSKLASKNDGSNYDLNEEINNISKVHTTYKSDKWYNPFKTWFTIKYDMRRLQKARNEITFNVGKDYNLLEDQKNFLLIHPELVLILDKQNYNGYLITPELVLMYCDVVEGRWNISACAKLDPKLQSMYQKGNNLWEVIDKLFPIMGEKTFDVISLLEPLALSLIQTHDPVKQLRGAFLNHVLSEMELIFESRESIKEFLSVDYIDKILDIFNKSTIDEIAEIFSFFRTFGHPPLEASIAAEKVRKYMYIEKQLKFDTINKCHAIFCTIIINGYRERHGGQWPPVTLPDHAHEFIINAYGSNSAISYENAVDYYQSFIGIKFNKFIEPQLDEDLTIYMKDKALSPKKSNWDTVYPASNLLYRTNASNESRRLVEVFIADSKFDPHQILDYVESGDWLDDPEFNISYSLKEKEIKQEGRLFAKMTYKMRATQVLSETLLANNIGKFFQENGMVKGEIELLKRLTTISISGVPRYNEVYNNSKSHTDDLKTYNKISNLNLSSNQKSKKFEFKSTDIYNDGYETVSCFLTTDLKKYCLNWRYESTALFGETCNQIFGLNKLFNWLHPRLEGSTIYVGDPYCPPSDKEHISLEDHPDSGFYVHNPRGGIEGFCQKLWTLISISAIHLAAVRIGVRVTAMVQGDNQAIAVTTRVPNNYDYRVKKEIVYKDVVRFFDSLREVMDDLGHELKLNETIISSKMFIYSKRIYYDGRILPQALKALSRCVFWSETVIDETRSASSNLATSFAKAIENGYSPVLGYACSIFKNIQQLYIALGMNINPTITQNIRDQYFRNPNWMQYASLIPASVGGFNYMAMSRCFVRNIGDPSVAALADIKRFIKANLLDRSVLYRIMNQEPGESSFLDWASDPYSCNLPQSQNITTMIKNITARNVLQDSPNPLLSGLFTNTMIEEDEELAEFLMDRKVILPRVAHDILDNSLTGIRNAIAGMLDTTKSLIRVGINRGGLTYSLLRKISNYDLVQYETLSRTLRLIVSDKIKYEDMCSVDLAIALRQKMWIHLSGGRMISGLETPDPLELLSGVVITGSEHCKICYSSDGTNPYTWMYLPGNIKIGSAETGISSLRVPYFGSVTDERSEAQLGYIKNLSKPAKAAIRIAMIYTWAFGNDEISWMEASQIAQTRANFTLDSLKILTPVATSTNLSHRLKDTATQMKFSSTSLIRVSRFITMSNDNMSIKEANETKDTNLIYQQIMLTGLSVFEYLFRLKETTGHNPIVMHLHIEDECCIKESFNDEHINPESTLELIRYPESNEFIYDKDPLKDVDLSKLMVIKDHSYTIDMNYWDDTDIIHAISICTAITIADTMSQLDRDNLKEIIVIANDDDINSLITEFLTLDILVFLKTFGGLLVNQFAYTLYSLKIEGRDLIWDYIMRTLRDTSHSILKVLSNALSHPKVFKRFWDCGVLNPIYGPNTASQDQIKLALSICEYSLDLFMREWLNGVSLEMYICDSDMEVANDRKQAFISRHLSFVCCLAEIASFGPNLLNLTYLERLDLLKQYLELNIKEDPTLKYVQISGLLIKSFPSTVTYVRKTAIKYLRIRGISPPEVIDDWDPVEDENMLDNIVKTINDNCNKDNKGNKINNFWGLALKNYQVLKIRSITSDSDDNDRLDANTSGLTLPQGGNYLSHQLRLFGINSTSCLKALELSQILMKEVNKDKDRLFLGEGAGAMLACYDATLGPAVNYYNSGLNITDVIGQRELKIFPSEVSLVGKKLGNVTQILNRVKVLFNGNPNSTWIGNMECESLIWSELNDKSIGLVHCDMEGAIGKSEETVLHEHYSVIRITYLIGDDDVVLVSKIIPTITPNWSRILYLYKLYWKDVSIISLKTSNPASTELYLISKDAYCTIMEPSEIVLSKLKRLSLLEENNLLKWIILSKKKNNEWLHHEIKEGERDYGVMRPYHMALQIFGFQINLNHLAKEFLSTPDLTNINNIIQSFQRTIKDVLFEWINITHDDKRHKLGGRYNIFPLKNKGKLRLLSRRLVLSWISLSLSTRLLTGRFPDEKFEHRAQTGYVSLADTDLESLKLLSKNVIKNYRECIGSISYWFLTKEVKILMKLIGGAKLLGIPRQYKEPEDQLLENYNQYDEFDID

SEQ ID NO: 10 - DNA, 25 bp, Human parainfluenza virus 3
aattataaaaaacttaggagtaaag

SEQ ID NO: 11 - DNA, 53 bp, Human parainfluenza virus 3
ccgacgtcttaattaatacgactcactataggaccaaacaagagaagaaactt

SEQ ID NO: 12 - DNA, 19 bp, Human parainfluenza virus 3
ggtcaccacaagagttaga

SEQ ID NO: 13 - DNA, 19 bp, Human parainfluenza virus 3
tctaactcttgtggtgacc

SEQ ID NO: 14 - DNA, 19 bp, Human parainfluenza virus 3
attcatcccaagggcaata

SEQ ID NO: 15 - DNA, 21 bp, Human parainfluenza virus 3
agaatggttattcacctgttc

SEQ ID NO: 16 - DNA, 20 bp, Human parainfluenza virus 3
gagaagcactctgtgtggta

SEQ ID NO: 17 - DNA, 35 bp, Human parainfluenza virus 3
cttaggagcaaagcgtgctcagaaaatggacactg

SEQ ID NO: 18 - DNA, 35 bp, Human parainfluenza virus 3
cagtgtccattttctgagcacgctttgctcctaag

SEQ ID NO: 19 - DNA, 60 bp, Human parainfluenza virus 3
tttttttttttttttttttttttttttttttttttttttttttttttttttttttttttt

SEQ ID NO: 20 - DNA, 23 bp, Human parainfluenza virus 3
tcgttttagatccttctcaatca

SEQ ID NO: 21 - DNA, 26 bp, Human parainfluenza virus 3
ggaaggagccatcggcaaatcagaag

SEQ ID NO: 22 - DNA, 79 bp, Human parainfluenza virus 3
tttttgtgcgcccaatacgcaaaccgcctctccccgcgcgttggccgttaattaagagggtgaccctgcacagagtgcc

SEQ ID NO: 23 - DNA, 79 bp, Human parainfluenza virus 3
tttttgtaaaaaacccctcaagacccgtttagaggccccaaggggttatgctagttaggtacccgggcactctgtgcag

SEQ ID NO: 24 - DNA, 67 bp, Human parainfluenza virus 3
accacacagagtgcttctcttgtttggtgggtcggcatggcatctccacctcctcgcggtccgacct

SEQ ID NO: 25 - DNA, 67 bp, Human parainfluenza virus 3
ggccggtacctcccttagccatccgagtggacgacgtcctccttcggatgcccaggtcggaccgcga

SEQ ID NO: 26 - DNA, 21 bp, Human parainfluenza virus 3
gtgaccgcgcatgcccacaga

SEQ ID NO: 27 - DNA, 13 bp, Human parainfluenza virus 3
gtgggcatgcgcg

SEQ ID NO: 28 - DNA, 42 bp, Human parainfluenza virus 3
ttgactagaaggtcaagaacctgcaggtcgactctagaggat

SEQ ID NO: 29 - DNA, 67 bp, Human parainfluenza virus 3
ttgaccttctagtcaatgtctttaatcctaagtttttcttatttattaaccggcgctcagttggaat

SEQ ID NO: 30 - DNA, 23 bp, Human parainfluenza virus 3
gaaggtcaagaaaagggaactct

SEQ ID NO: 31 - DNA, 22 bp, Human parainfluenza virus 3
ttgattcgattagttgcttcca

SEQ ID NO: 32 - DNA, 20 bp, Human parainfluenza virus 3
tgatggaaagcgacgctaaa

SEQ ID NO: 33 - DNA, 20 bp, Human parainfluenza virus 3
ggatcattggcaattgttga

SEQ ID NO: 34 - DNA, 20 bp, Human parainfluenza virus 3
gcgtgctcagaaaatggaca

SEQ ID NO: 35 - DNA, 27 bp, Human parainfluenza virus 3
ccttaggcttaaagataaaggttagga

SEQ ID NO: 36 - DNA, 25 bp, Human parainfluenza virus 3
aattataaaaaacttaggagtaaag

SEQ ID NO: 37 - DNA, 26 bp, Human parainfluenza virus 3
cgttatagtgctgccacaaagaataa

SEQ ID NO: 38 - DNA, 21 bp, Human parainfluenza virus 3
atggaagaccagacgtgcatc

SEQ ID NO: 39 - DNA, 28 bp, Human parainfluenza virus 3
cgattaaggaaagcgacctgtaagtaat

SEQ ID NO: 40 - DNA, 23 bp, Human parainfluenza virus 3
gagacacaaattaggcgggagat

SEQ ID NO: 41 - DNA, 16471 bp, Human parainfluenza virus 3
taatacgactcactataggaccaaacaagagaagaaacttgtcaggaaatataaatttaacttaaaattaacttaggattaaagacattgactagaaggtcaagaacctgcaggtcgactctagaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattccaactgagcgccggttaataaataagaaaaacttaggattaaagacattgactagaaggtcaagaaaagggaactctataatttcaaaaatgttgagcctatttgatacatttaatgcacgtaggcaagaaaacataacaaaatcagctggtggagctatcattcctggacagaaaaatactgtctctatattcgcccttggaccgacaataactgatgataatgagaaaatgacattagctcttctatttctgtctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaggatctaaaacgacaaaagtatggaggatttgtggttaagacgagagagatgatatatgaaaagacaactgattggatatttggaagtgacctggattatgatcaggaaactatgttgcagaacggcagaaacaattcaacaattgaggaccttgtccacacatttgggtatccatcatgtttaggagctcttataatacagatctggatagttttagtcaaagctatcactagtatctcaggattaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagactagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgctgaagctcaaatgagctcaacactggaagatgaacttggagtgacacacgaagctaaagaaagcttgaagagacatataaggaacataaacagttcagagacatctttccacaaaccaacaggtggatcagccatagagatggcaatagatgaagagccagaacaattcgaacatagagcagatcaagaacagaatggagaacctcaatcatccataattcaatatgcctgggcagaaggaaatagaagcgatgatcagactgagcaggctacagaatctgacaatatcaagaccgaacaacaaaacatcagagacagactaaacaagagactcaatgacaagaagaaacaaagcagtcaaccacctaccaatcccacaaacagaacaaaccaggacgaaatagatgatctgtttaatgcatttggaagcaactaatcgaatcaacgttttaatccaaatcaataataaataagaaaaacttaggattaaagaatcctatcataccggaatatagagcggtaaatttagagtctgcttgcaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacctatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctagaggaatccccagaagcatcacagattctaaaaatggaacccaaaacacggaggatattgatctcaatgaaattagaaagatggataaggactctattgaggggaaaatgcgacaatctgcaaatgttccaagcgaggtatcaggaagtgatgacatacttacaacagaacaaagtagaaacagtgatcatggaagaagcctggaatctatcagtacacctgatacaagatcaataagtgttgttactgctgcaacaccagatgatgaagaagaaatactaatgaaaaacagtaggacaaagaaaagttcttcaacacatcaagaagatgacaaaagaattaaaaaagggggaaaagggaaagactggtttaagaaatcaaaagatactgacaaccagataccaacatcagactacagatccacatcaaaagggcagaagaaaatctcaaagacaacaaccatcaacaccgacacaaaggggcaaacagaaatacagacagaatcatcagaaacacaatcttcatcatggaatctcatcatcgacaacaacaccgaccgaaacgaacagacaagcacaactcctccaacaacaacttcaagatcaacctatacaaaagaatcgatccgaacaaactctgaatccaaacccaagacacaaaagacaaatggaaaggaaaggaaggatacagaagagagcaatcgatttacagagagggcaattactctattgcagaatcttggtgtaatccaatctacatcaaaattagatttatatcaagacaaacgagttgtatgtgtagcaaatgtactaaacaatgtagatactgcatcaaagatagacttcctggcaggattagtcataggggtttcaatggataacgacacaaaattaacacagatacaaaatgaaatgctaaacctcaaaacagatctaaagaaaatggacgaatcacatagaagattgatagaaaatcaaagagaacaactgtcattgatcacgtcattaatttcaaatcttaaaattatgactgagagaggaggtaagaaagaccaaaatgaatccactgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatactaagttcatataatgagtcaaatgcaacaagactaatacccaaaaaagtgagcagtacaatgagatcactagttgcagtcatcaacaacagcaacctctcacaaagcacaaaacaatcatacataaacgaactcaaacgttgcaaaaatgatgaagaagtatctgaattaatggacatgttcaatgaagatgtcaacaattgccaatgatccaacaaagaaactacactgaacaaacagacaagaaacaacagcagatcaaaatctgtcaacacacacaaaatcaagcagaataaaacaacagatatcaatcaacatacaaataagaaaaacttaggattaaagaataaattaatccttgtccaaaatgagtataactaactctgcaatatacacattcccagaatcatcattctttgaaaatggtcatatagaaccattaccactcaaagtcaatgaacagagaaaagcagtaccccacattagagttgccaaaatcggaaatccaccaaaacacggatcccggtatttagatgtcttcttacttggctttttcgagatggaacgaatcaaagacaaatacgggagtgtgaatgatctcgacagtgacccgagttacaaagtctgtggctctggatcattaccaatcggattggctaagtacactgggaatgaccaagaattgttacaagctgcaaccaaactggacatagaagtgagaagaacagttaaagcgaaagagatggttgtttacacggtacaaaatataaaaccagaactgtacccatggtccaatagactaagaaaaggaatgctgttcgatgccaacaaagttgctcttgctcctcaatgtcttccactagataggagcataaaattcagagtaatcttcgtgaattgtacagcaattggatcaataaccctgttcaaaattcccaagtcaatggcatcactatctctacccaacacaatatcaatcaatctgcaggtacacatcaaaacaggggttcagactgattctaaagggatagttcaaattttggatgagaaaggagaaaaatcactgaatttcatggtccatctcggattgattaaaagaaaagtaggcagaatgtactctgttgaatactgtaaacagaaaatcgagaaaatgagattgatattttctttaggactagttggaggaatcagtcttcatgtcaatgcaaccggatccatatcaaaaacactagcaagtcagctggtattcaagagggagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctatttcagtccagacgtatctattaagctgaagcaaataagggataatcaaaaacttaggataaaagaggtcaataccaacaaccattagcagtcatactcgcaagaataagaaaggagggatttaaaaagttaaatagaggaaatcaaaacaaaaagtacagaacaccagaacaataaaatcaaaacatccaactcactcaaaacaaaaatcccaaaagagaccagtaatacaacaagcactgagcacaatgacaacttcaatactgctaattattacaaccatgatcatggcatctttctgccaaatagatatcacaaaactacagcatgtaggtgtattggtcaacagtcccaaagggatgaagatatcacaaaactttgaaacaagatatctgattttgagcctcataccaaaaatagaagattctaactcttgtggtgaccagcagatcaagcaatacaagaagctattggatagactgatcatccctttatatgatggattaagattacagaaagatgtgatagtaaccaatcaagaatccaatgaaaacactgatcctagaacaaaacgattctttggaggggtaattggaactattgctctgggagtagcaacctcagcacaaattacagcggcagttgctttggttgaagccaagcaggcaagatcagacatcgaaaaactcaaagaagcaattagggacacaaataaagcagtgcagtcagttcagagctccataggaaatctaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatatttacaacatcaacagttgacaaatatgatatttatgatctgttatttacagaatcaataaaagtgagagttatagatgttgacttgaatgattactcaatcactctccaagtcagactccctttattaactaggctgctgaacactcagatctacaaagtagattccatatcatataacatccaaaacagagaatggtatatccctcttcccagccatattatgacgaaaggggcatttctaggtggagcagatgtcaaagaatgtatagaagcattcagcagctatatatgcccttctgatccaggatttgtattaaaccatgaaatagagagctgcttatcaggaaacatatctcaatgtccaagaaccacagtcacatcagacattgttccaagatatgcatttgtcaatggaggagtggttgcaaactgtataacaaccacttgtacatgcaatggaatcggtaatagaatcaatcaaccacctgatcaaggaataaaaattataacacataaagaatgtagtacaataggtatcaacggaatgctgttcaatacaaataaagaaggaactcttgcattctacacaccaaatgatataacactaaacaattctgttgcacttgatccaattgacatatcaattgagctcaacaaggccaaatcagatctagaagaatcaaaagaatggataagaaggtcaaatcaaaaactagattccattggaaattggcaccaatctagcactacaatcataattattttgataatgatcattatattgtttataattaatgtaacgataattacaattgcaattaagtattacagaattcaaaagagaaatcgagtggatcaaaatgacaagccgtatgtactgacaaacaaataatatatctacagatcattagatattaaaattataaaaaacttaggagtaaagttacacaatccaactctactcatataattgaggaaaaacctaatagacaaatccaaattcgagatggaatactggaagcataccaatcacggaaaggatgctggtaatgagctggagacgtccatggctactcatggcaacaagctcaccaataagataatatacatattatggacaataatcctggtgttattatcaatagtcttcatcatagtgctaaccaattccatcaaaagtgaaaagacccatgaatcattgctgcgagacataaacaatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcatcagtgaaattataattagaaatgataatcaagaagtgctgccacaaagaataacgcatgatgtaggtataaaacctttaaatccagatgatttttggagatgcacgtctggtcttccatctttaatgaaaactccaaaaataaggttaatgccagggccggggttattagctatgccaacgactgttgatggctgtattagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatactttcaacataaatgacaataggaagtcatgctctctagcactcctaaatacagatgtatatcaactgtgttcaactcccaaagttgatgaaagatcagattatgcatcatcgggcatagaagatattgtacttgatattgtcaattatgatggctcaatctcaacaacaagatttaagaataataacataagctttgatcaaccatatgctgcgctatacccatctgttggaccagggatatactacaaaggcaaaataatatttcttggatatggaggtcttgaacacccaataaatgagaatgtgatctgcaacacaactgggtgtcccgggaaaacacagagagactgtaatcaagcgtctcatagtccatggttttcagataggaggatggtcaactccatcattgttgttgacaaaggcttaaactcaactccaaaattgaaggtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccattcgaaacagaacactctcagctggatatacaacaacaagctgcattacacactataacaaaggatattgttttcatatagtagaaataaatcataaaagctcaaacacatttcaacccatgttgttcaaaacagagattccaaaaagctgcagttaatcataattaaccataatatgcattaatctatctacaacacaagtatattataagtaatcagcaatcagacaatagacaaaagggaaatataaaaaacttaggagcaaagcgtgctcagaaaatggacactgaatctaacaatggtactgtatctgacatactctatcctgagtgtcaccttaactctcctatcgttaaaggtaaaatagcacaattacacactattatgagtttacctcagccctatgatatggatgacgactcaatactagttatcactagacagaaaataaaactcaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcaaaagttcacacaacttataaatcagataaatggtataatccattcaaaacatggtttactattaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattatttccaattatgggagaaaagacatttgatgttatatcattattagaaccacttgcattatccttaattcaaactcatgatcctgttaaacaactaagaggagcttttttaaatcatgtgttatccgagatggaattaatatttgaatctagagaatcgattaaggaatttctgagtgtagattatattgataaaattttagatatatttaataaatctacaatagatgaaatagcagagattttctctttttttagaacatttgggcatcctccattagaagctagtattgcagcagaaaaggtcagaaaatatatgtatattgagaaacaattaaaatttgacactatcaataaatgtcatgctatcttctgtacaataataattaatggatatagagagagacatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaagatttgacaatttatatgaaagataaagcattatctccaaaaaaatcaaattgggatacagtttatcctgcatctaatttactgtaccgtactaacgcatccaacgaatcacgaagattagttgaagtatttatagcagatagtaaatttgatcctcatcaaatattggattatgtagaatctggggactggttagatgatccagaatttaatatttcttatagtcttaaagaaaaagagatcaaacaagaaggtagactctttgcaaaaatgacatacaaaatgagagctacacaagttttatcagagacactacttgcaaataacataggaaaattctttcaagaaaatgggatggtgaagggagagattgaattacttaagagattaacgaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttgggttaaataaattgtttaattggttacatcctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcgatacatctagcagctgttagaataggtgtgagggtgactgcaatggttcaaggagacaatcaagccatagctgtaactacaagagtacccaacaattatgactacagagttaagaaggagatagtttataaagatgtagtgagattttttgattcattaagagaagttatggatgatctaggtcatgagcttaaattaaatgaaacgattataagtagcaagatgttcatatatagcaaaagaatctattatgatgggagaattcttcctcaagctctgaaagcattatctagatgtgtcttctggtcagagacagtaatagacgaaacaagatcagcatcttcaaacttggcaacatcatttgcaaaagcaattgagaatggttattcacctgttttaggatatgcatgctcaatttttaagaatattcaacaactatatattgcccttgggatgaatattaatccaactataacacagaatatcagagatcagtattttaggaatccaaattggatgcaatatgcctctttaatacctgctagtgttgggggattcaattacatggcaatgtcaagatgttttgtaaggaatattggtgatccatcagttgccgcattagctgatattaaaagatttattaaggcgaatctattagaccgaagtgttctttataggattatgaatcaagaaccaggtgagtcatcttttttggattgggcttcagatccatattcatgcaatttaccacaatctcaaaatataaccactatgataaaaaatataacagcaaggaatgtattacaagattcaccaaatccgttattatctggattattcacaaatacaatgatagaagaagatgaagaattagctgagttcctgatggacaggaaggtaattcttcctagagttgcacatgatattctagataattctcttacaggaattagaaatgccatagctggaatgttagatacgacaaaatcactaattcgagttggcataaatagagggggactgacatatagtttgttgaggaaaatcagtaattatgatctagtacaatatgaaacactaagtaggaccttgcgattaattgtaagcgataaaatcaagtatgaagatatgtgttcagtagaccttgccatagcattgcgacaaaaaatgtggattcatttatcaggaggaaggatgataagtggacttgaaacgcctgacccattagaactactatctggggtagtaataacaggatcagaacattgtaaaatatgttattcttcagatggaacaaacccatatacttggatgtatttacccggtaatatcaaaataggatcagcagaaacaggtatatcatcattaagagttccttactttggatcagtcactgatgaaagatctgaggcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttgacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagataccaatcttatttatcaacaaataatgttaacaggattaagtgttttcgaatatttatttagattaaaagaaaccacaggacataaccctatagttatgcatctgcacatagaagatgagtgttgtattaaagaaagttttaatgatgaacatattaatccagaatctacattagaattaattcgatatcctgaaagtaatgaatttatttatgataaagacccactcaaagatgtggacttatcaaaacttatggttattaaagaccattcttacacaattgatatgaattattgggatgataccgatatcatacatgcaatttcaatatgtactgcaattacaatagcagatactatgtcacaattagatcgagataatttaaaagagataatagtcattgcaaatgatgatgatattaatagcttaatcactgaatttttgactcttgacatacttgtatttcttaagacatttggtggattattagtaaatcaatttgcatacactctttatagtttaaaaatagaaggtagggatctcatttgggattatataatgagaacactgagagatacttcccattcaatattaaaagtattatctaatgcattatctcatcctaaggtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatgtacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgtggtattagtccacctgaagtaattgatgattgggatccggtagaagatgaaaatatgctggataacattgtcaaaactataaatgataactgcaataaagataataaagggaataaaattaacaatttctggggactagcactcaagaactatcaagtccttaaaatcagatctataacaagtgattctgatgataatgatagactagatgctaatacaagtggtttgacacttccccaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggcaaaaaattaggaaatgtgacacagattcttaacagggtaaaagtactgttcaatgggaatcccaattcaacatggataggaaatatggaatgtgagagcttgatatggagtgaattaaatgataagtctattggattagtacattgtgatatggaaggagccatcggcaaatcagaagaaactgttctacatgaacattatagtgttataagaattacatacttgattggggatgatgatgttgttttagtttccaagattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaagaataatgaatggttacatcatgaaatcaaagaaggagaaagagattatggagtcatgagaccatatcatatggcactacaaatctttggatttcaaatcaatttaaatcatctagcgaaagaatttttatcgaccccagatctgactaatatcaacaatataatccaaagttttcagcggacaatcaaggatgttttatttgaatggatcaatataactcatgatgataagagacacaaattaggcgggagatataacatattcccactgaaaaataagggaaagttaagactgctatcaagaagactagtattaagttggatttcattatcattatcgactcgattacttacaggtcgctttcctgatgaaaaatttgaacatagagcacagacaggatatgtatcattagctgatactgatttagaatcattaaagttattgtcgaaaaacgtcattaagaattacagagagtgtataggatcaatatcatattggtttctaactaaggaagttaaaatacttatgaaattgattggtggtgctaaattattaggaattcccagacaatataaagaacccgaagatcagttattagaaaactacaatcaatatgatgaatttgatatcgattaaaacataaatacaatgaagatatatcctaacctttatctttaagcctaaggatagacaaaaagtaagaaaaacatgtaatatatatataccacacagagtgcttctcttgtttggtgggtcggcatggcatctccacctcctcgcggtccgacctgggcatccgaaggaggacgtcgtccactcggatggctaagggaggtacctaactagcataaccccttggggcctctaaacgggtcttgaggggtttttt

SEQ ID NO: 42 - RNA, 16314 nt, Human parainfluenza virus 3
accaaacaagagaagaaacuugucaggaaauauaaauuuaacuuaaaauuaacuuaggauuaaagacauugacuagaaggucaagaaccugcaggucgacucuagaggauccccggguaccggucgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaaagcggccgcgacucuagaauuccaacugagcgccgguuaauaaauaagaaaaacuuaggauuaaagacauugacuagaaggucaagaaaagggaacucuauaauuucaaaaauguugagccuauuugauacauuuaaugcacguaggcaagaaaacauaacaaaaucagcugguggagcuaucauuccuggacagaaaaauacugucucuauauucgcccuuggaccgacaauaacugaugauaaugagaaaaugacauuagcucuucuauuucugucucauucacuagauaaugagaaacaacaugcacaaagggcaggguucuuggugucuuuauugucaauggcuuaugccaauccagagcucuaccuaacaacaaauggaaguaaugcagaugucaaguaugucauauacaugauugagaaggaucuaaaacgacaaaaguauggaggauuugugguuaagacgagagagaugauauaugaaaagacaacugauuggauauuuggaagugaccuggauuaugaucaggaaacuauguugcagaacggcagaaacaauucaacaauugaggaccuuguccacacauuuggguauccaucauguuuaggagcucuuauaauacagaucuggauaguuuuagucaaagcuaucacuaguaucucaggauuaagaaaaggcuuuuucacccgauuggaagcuuucagacaagauggaacagugcaggcagggcugguauugagcggugacacaguggaucagauugggucaaucaugcggucucaacagagcuugguaacucuuaugguugaaacauuaauaacaaugaauaccagcagaaaugaccucacaaccauagaaaagaauauacaaauuguuggcaacuacauaagagaugcaggucucgcuucauucuucaauacaaucagauauggaauugagacuagaauggcagcuuugacucuauccacucucagaccagauaucaauagauuaaaagcuuugauggaacuguauuuaucaaagggaccacgcgcuccuuucaucuguauccucagagauccuauacauggugaguucgcaccaggcaacuauccugccauauggagcuaugcaauggggguggcaguuguacaaaauagagccaugcaacaguaugugacgggaagaucauaucuagacauugauauguuccagcuaggacaagcaguagcacgugaugcugaagcucaaaugagcucaacacuggaagaugaacuuggagugacacacgaagcuaaagaaagcuugaagagacauauaaggaacauaaacaguucagagacaucuuuccacaaaccaacagguggaucagccauagagauggcaauagaugaagagccagaacaauucgaacauagagcagaucaagaacagaauggagaaccucaaucauccauaauucaauaugccugggcagaaggaaauagaagcgaugaucagacugagcaggcuacagaaucugacaauaucaagaccgaacaacaaaacaucagagacagacuaaacaagagacucaaugacaagaagaaacaaagcagucaaccaccuaccaaucccacaaacagaacaaaccaggacgaaauagaugaucuguuuaaugcauuuggaagcaacuaaucgaaucaacguuuuaauccaaaucaauaauaaauaagaaaaacuuaggauuaaagaauccuaucauaccggaauauagagcgguaaauuuagagucugcuugcaacucaaucaauagagaguugauggaaagcgaugcuaaaaacuaucaaaucauggauucuugggaagaggaaucaagagauaaaucaacuaauaucuccucggcccucaacaucauugaauucauacucagcaccgacccccaagaagaccuaucggaaaacgacacaaucaacacaagaacccagcaacucagugccaccaucugucaaccagaaaucaaaccaacagaaacaagugagaaagauaguggaucaacugacaaaaauagacagucugggucaucacacgaauguacaacagaagcaaaagauagaaauauugaucaggaaacuguacagagaggaccugggagaagaagcagcucagauaguagagcugagacuguggucucuagaggaauccccagaagcaucacagauucuaaaaauggaacccaaaacacggaggauauugaucucaaugaaauuagaaagauggauaaggacucuauugaggggaaaaugcgacaaucugcaaauguuccaagcgagguaucaggaagugaugacauacuuacaacagaacaaaguagaaacagugaucauggaagaagccuggaaucuaucaguacaccugauacaagaucaauaaguguuguuacugcugcaacaccagaugaugaagaagaaauacuaaugaaaaacaguaggacaaagaaaaguucuucaacacaucaagaagaugacaaaagaauuaaaaaagggggaaaagggaaagacugguuuaagaaaucaaaagauacugacaaccagauaccaacaucagacuacagauccacaucaaaagggcagaagaaaaucucaaagacaacaaccaucaacaccgacacaaaggggcaaacagaaauacagacagaaucaucagaaacacaaucuucaucauggaaucucaucaucgacaacaacaccgaccgaaacgaacagacaagcacaacuccuccaacaacaacuucaagaucaaccuauacaaaagaaucgauccgaacaaacucugaauccaaacccaagacacaaaagacaaauggaaaggaaaggaaggauacagaagagagcaaucgauuuacagagagggcaauuacucuauugcagaaucuugguguaauccaaucuacaucaaaauuagauuuauaucaagacaaacgaguuguauguguagcaaauguacuaaacaauguagauacugcaucaaagauagacuuccuggcaggauuagucauagggguuucaauggauaacgacacaaaauuaacacagauacaaaaugaaaugcuaaaccucaaaacagaucuaaagaaaauggacgaaucacauagaagauugauagaaaaucaaagagaacaacugucauugaucacgucauuaauuucaaaucuuaaaauuaugacugagagaggagguaagaaagaccaaaaugaauccacugagagaguauccaugaucaaaacaaaauugaaagaagaaaagaucaagaagaccagguuugacccacuuauggaggcacaaggcauugacaagaauauacccgaucuauaucgacaugcaggagauacacuagagaacgauguacaaguuaaaucagagauacuaaguucauauaaugagucaaaugcaacaagacuaauacccaaaaaagugagcaguacaaugagaucacuaguugcagucaucaacaacagcaaccucucacaaagcacaaaacaaucauacauaaacgaacucaaacguugcaaaaaugaugaagaaguaucugaauuaauggacauguucaaugaagaugucaacaauugccaaugauccaacaaagaaacuacacugaacaaacagacaagaaacaacagcagaucaaaaucugucaacacacacaaaaucaagcagaauaaaacaacagauaucaaucaacauacaaauaagaaaaacuuaggauuaaagaauaaauuaauccuuguccaaaaugaguauaacuaacucugcaauauacacauucccagaaucaucauucuuugaaaauggucauauagaaccauuaccacucaaagucaaugaacagagaaaagcaguaccccacauuagaguugccaaaaucggaaauccaccaaaacacggaucccgguauuuagaugucuucuuacuuggcuuuuucgagauggaacgaaucaaagacaaauacgggagugugaaugaucucgacagugacccgaguuacaaagucuguggcucuggaucauuaccaaucggauuggcuaaguacacugggaaugaccaagaauuguuacaagcugcaaccaaacuggacauagaagugagaagaacaguuaaagcgaaagagaugguuguuuacacgguacaaaauauaaaaccagaacuguacccaugguccaauagacuaagaaaaggaaugcuguucgaugccaacaaaguugcucuugcuccucaaugucuuccacuagauaggagcauaaaauucagaguaaucuucgugaauuguacagcaauuggaucaauaacccuguucaaaauucccaagucaauggcaucacuaucucuacccaacacaauaucaaucaaucugcagguacacaucaaaacagggguucagacugauucuaaagggauaguucaaauuuuggaugagaaaggagaaaaaucacugaauuucaugguccaucucggauugauuaaaagaaaaguaggcagaauguacucuguugaauacuguaaacagaaaaucgagaaaaugagauugauauuuucuuuaggacuaguuggaggaaucagucuucaugucaaugcaaccggauccauaucaaaaacacuagcaagucagcugguauucaagagggagauuuguuauccuuuaauggaucuaaauccgcaucucaaucuaguuaucugggcuucaucaguagagauuacaagaguggaugcaauuuuccaaccuucuuuaccuggcgaguucagauacuauccuaauauuauugcaaaaggaguugggaaaaucaaacaauggaacuaguaaucucuauuucaguccagacguaucuauuaagcugaagcaaauaagggauaaucaaaaacuuaggauaaaagaggucaauaccaacaaccauuagcagucauacucgcaagaauaagaaaggagggauuuaaaaaguuaaauagaggaaaucaaaacaaaaaguacagaacaccagaacaauaaaaucaaaacauccaacucacucaaaacaaaaaucccaaaagagaccaguaauacaacaagcacugagcacaaugacaacuucaauacugcuaauuauuacaaccaugaucauggcaucuuucugccaaauagauaucacaaaacuacagcauguagguguauuggucaacagucccaaagggaugaagauaucacaaaacuuugaaacaagauaucugauuuugagccucauaccaaaaauagaagauucuaacucuuguggugaccagcagaucaagcaauacaagaagcuauuggauagacugaucaucccuuuauaugauggauuaagauuacagaaagaugugauaguaaccaaucaagaauccaaugaaaacacugauccuagaacaaaacgauucuuuggagggguaauuggaacuauugcucugggaguagcaaccucagcacaaauuacagcggcaguugcuuugguugaagccaagcaggcaagaucagacaucgaaaaacucaaagaagcaauuagggacacaaauaaagcagugcagucaguucagagcuccauaggaaaucuaauaguagcaauuaaaucaguccaggauuauguuaacaaagaaaucgugccaucgauugcgaggcuagguugugaagcagcaggacuucaauuaggaauugcauuaacacagcauuacucagaauuaacaaacauauuuggugauaacauaggaucguuacaagaaaaaggaauaaaauuacaagguauagcaucauuauaccgcacaaauaucacagaaauauuuacaacaucaacaguugacaaauaugauauuuaugaucuguuauuuacagaaucaauaaaagugagaguuauagauguugacuugaaugauuacucaaucacucuccaagucagacucccuuuauuaacuaggcugcugaacacucagaucuacaaaguagauuccauaucauauaacauccaaaacagagaaugguauaucccucuucccagccauauuaugacgaaaggggcauuucuagguggagcagaugucaaagaauguauagaagcauucagcagcuauauaugcccuucugauccaggauuuguauuaaaccaugaaauagagagcugcuuaucaggaaacauaucucaauguccaagaaccacagucacaucagacauuguuccaagauaugcauuugucaauggaggagugguugcaaacuguauaacaaccacuuguacaugcaauggaaucgguaauagaaucaaucaaccaccugaucaaggaauaaaaauuauaacacauaaagaauguaguacaauagguaucaacggaaugcuguucaauacaaauaaagaaggaacucuugcauucuacacaccaaaugauauaacacuaaacaauucuguugcacuugauccaauugacauaucaauugagcucaacaaggccaaaucagaucuagaagaaucaaaagaauggauaagaaggucaaaucaaaaacuagauuccauuggaaauuggcaccaaucuagcacuacaaucauaauuauuuugauaaugaucauuauauuguuuauaauuaauguaacgauaauuacaauugcaauuaaguauuacagaauucaaaagagaaaucgaguggaucaaaaugacaagccguauguacugacaaacaaauaauauaucuacagaucauuagauauuaaaauuauaaaaaacuuaggaguaaaguuacacaauccaacucuacucauauaauugaggaaaaaccuaauagacaaauccaaauucgagauggaauacuggaagcauaccaaucacggaaaggaugcugguaaugagcuggagacguccauggcuacucauggcaacaagcucaccaauaagauaauauacauauuauggacaauaauccugguguuauuaucaauagucuucaucauagugcuaaccaauuccaucaaaagugaaaagacccaugaaucauugcugcgagacauaaacaaugaguuuauggaaauuacagaaaagauccaaauggcaucggauaauaccaaugaucuaauacagucaggagugaauacaaggcuucuuacaauucagagucauguccagaauuacauaccaauaucauugacacaacagaugucagaucuuaggaaauucaucagugaaauuauaauuagaaaugauaaucaagaagugcugccacaaagaauaacgcaugauguagguauaaaaccuuuaaauccagaugauuuuuggagaugcacgucuggucuuccaucuuuaaugaaaacuccaaaaauaagguuaaugccagggccgggguuauuagcuaugccaacgacuguugauggcuguauuagaacuccgucuuuaguuauaaaugaucugauuuaugcuuauaccucaaaucuaauuacucgagguugucaggauauaggaaaaucauaucaagucuuacagauagggauaauaacuguaaacucagacuugguaccugacuuaaauccuaggaucucucauacuuucaacauaaaugacaauaggaagucaugcucucuagcacuccuaaauacagauguauaucaacuguguucaacucccaaaguugaugaaagaucagauuaugcaucaucgggcauagaagauauuguacuugauauugucaauuaugauggcucaaucucaacaacaagauuuaagaauaauaacauaagcuuugaucaaccauaugcugcgcuauacccaucuguuggaccagggauauacuacaaaggcaaaauaauauuucuuggauauggaggucuugaacacccaauaaaugagaaugugaucugcaacacaacugggugucccgggaaaacacagagagacuguaaucaagcgucucauaguccaugguuuucagauaggaggauggucaacuccaucauuguuguugacaaaggcuuaaacucaacuccaaaauugaagguauggacgauaucuaugcgacaaaauuacugggggucagaaggaagguuacuucuacuagguaacaagaucuauauauauacaagaucuacaaguuggcauagcaaguuacaauuaggaauaauugauauuacugauuacagugauauaaggauaaaauggacauggcauaaugugcuaucaagaccaggaaacaaugaauguccauggggacauucauguccagauggauguauaacaggaguauauacugaugcauauccacucaaucccacagggagcauugugucaucugucauauuagacucacaaaaaucgagagugaacccagucauaacuuacucaacagcaaccgaaagaguaaacgagcuggccauucgaaacagaacacucucagcuggauauacaacaacaagcugcauuacacacuauaacaaaggauauuguuuucauauaguagaaauaaaucauaaaagcucaaacacauuucaacccauguuguucaaaacagagauuccaaaaagcugcaguuaaucauaauuaaccauaauaugcauuaaucuaucuacaacacaaguauauuauaaguaaucagcaaucagacaauagacaaaagggaaauauaaaaaacuuaggagcaaagcgugcucagaaaauggacacugaaucuaacaaugguacuguaucugacauacucuauccugagugucaccuuaacucuccuaucguuaaagguaaaauagcacaauuacacacuauuaugaguuuaccucagcccuaugauauggaugacgacucaauacuaguuaucacuagacagaaaauaaaacucaauaaauuggauaaaagacaacgaucuauuagaagauuaaaauuaauauuaacugaaaaagugaaugacuuaggaaaauacacauuuaucagauauccagaaaugucaaaagaaauguucaaauuauauauaccugguauuaacaguaaagugacugaauuauuacuuaaagcagauagaacauauagucaaaugacugauggauuaagagaucuauggauuaaugugcuaucaaaauuagccucaaaaaaugauggaagcaauuaugaucuuaaugaagaaauuaauaauauaucaaaaguucacacaacuuauaaaucagauaaaugguauaauccauucaaaacaugguuuacuauuaaguaugauaugagaagauuacaaaaagcucgaaaugagaucacuuuuaauguugggaaggauuauaacuuguuagaagaccagaagaauuucuuauugauacauccagaauugguuuugauauuagauaaacaaaacuauaaugguuaucuaauuacuccugaauuaguauugauguauugugacguagucgaaggccgauggaauauaagugcaugugcuaaguuagauccaaaauuacaaucuauguaucagaaagguaauaaccugugggaagugauagauaaauuauuuccaauuaugggagaaaagacauuugauguuauaucauuauuagaaccacuugcauuauccuuaauucaaacucaugauccuguuaaacaacuaagaggagcuuuuuuaaaucauguguuauccgagauggaauuaauauuugaaucuagagaaucgauuaaggaauuucugaguguagauuauauugauaaaauuuuagauauauuuaauaaaucuacaauagaugaaauagcagagauuuucucuuuuuuuagaacauuugggcauccuccauuagaagcuaguauugcagcagaaaaggucagaaaauauauguauauugagaaacaauuaaaauuugacacuaucaauaaaugucaugcuaucuucuguacaauaauaauuaauggauauagagagagacaugguggacaguggccuccugugacauuaccugaucaugcacacgaauucaucauaaaugcuuacgguucaaacucugcgauaucauaugaaaaugcuguugauuauuaccagagcuuuauaggaauaaaauucaauaaauucauagagccucaguuagaugaagauuugacaauuuauaugaaagauaaagcauuaucuccaaaaaaaucaaauugggauacaguuuauccugcaucuaauuuacuguaccguacuaacgcauccaacgaaucacgaagauuaguugaaguauuuauagcagauaguaaauuugauccucaucaaauauuggauuauguagaaucuggggacugguuagaugauccagaauuuaauauuucuuauagucuuaaagaaaaagagaucaaacaagaagguagacucuuugcaaaaaugacauacaaaaugagagcuacacaaguuuuaucagagacacuacuugcaaauaacauaggaaaauucuuucaagaaaaugggauggugaagggagagauugaauuacuuaagagauuaacgaccauaucaauaucaggaguuccacgguauaaugaaguguacaauaauucuaaaagccauacagaugaccuuaaaaccuacaauaaaauaaguaaucuuaauuugucuucuaaucagaaaucaaagaaauuugaauucaagucaacggauaucuacaaugauggauacgagacugugagcuguuuccuaacaacagaucucaaaaaauacugucuuaauuggagauaugaaucaacagcucuauuuggagaaacuugcaaccaaauauuuggguuaaauaaauuguuuaauugguuacauccucgucuugaaggaaguacaaucuauguaggugauccuuacuguccuccaucagauaaagaacauauaucauuagaggaucacccugauucugguuuuuacguucauaacccaagaggggguauagaaggauuuugucaaaaauuauggacacucauaucuauaagugcgauacaucuagcagcuguuagaauaggugugagggugacugcaaugguucaaggagacaaucaagccauagcuguaacuacaagaguacccaacaauuaugacuacagaguuaagaaggagauaguuuauaaagauguagugagauuuuuugauucauuaagagaaguuauggaugaucuaggucaugagcuuaaauuaaaugaaacgauuauaaguagcaagauguucauauauagcaaaagaaucuauuaugaugggagaauucuuccucaagcucugaaagcauuaucuagaugugucuucuggucagagacaguaauagacgaaacaagaucagcaucuucaaacuuggcaacaucauuugcaaaagcaauugagaaugguuauucaccuguuuuaggauaugcaugcucaauuuuuaagaauauucaacaacuauauauugcccuugggaugaauauuaauccaacuauaacacagaauaucagagaucaguauuuuaggaauccaaauuggaugcaauaugccucuuuaauaccugcuaguguugggggauucaauuacauggcaaugucaagauguuuuguaaggaauauuggugauccaucaguugccgcauuagcugauauuaaaagauuuauuaaggcgaaucuauuagaccgaaguguucuuuauaggauuaugaaucaagaaccaggugagucaucuuuuuuggauugggcuucagauccauauucaugcaauuuaccacaaucucaaaauauaaccacuaugauaaaaaauauaacagcaaggaauguauuacaagauucaccaaauccguuauuaucuggauuauucacaaauacaaugauagaagaagaugaagaauuagcugaguuccugauggacaggaagguaauucuuccuagaguugcacaugauauucuagauaauucucuuacaggaauuagaaaugccauagcuggaauguuagauacgacaaaaucacuaauucgaguuggcauaaauagagggggacugacauauaguuuguugaggaaaaucaguaauuaugaucuaguacaauaugaaacacuaaguaggaccuugcgauuaauuguaagcgauaaaaucaaguaugaagauauguguucaguagaccuugccauagcauugcgacaaaaaauguggauucauuuaucaggaggaaggaugauaaguggacuugaaacgccugacccauuagaacuacuaucugggguaguaauaacaggaucagaacauuguaaaauauguuauucuucagauggaacaaacccauauacuuggauguauuuacccgguaauaucaaaauaggaucagcagaaacagguauaucaucauuaagaguuccuuacuuuggaucagucacugaugaaagaucugaggcacaauuaggauauaucaagaaucuuaguaaaccugcaaaagccgcaauaagaauagcaaugauauauacaugggcauuugguaaugaugagauaucuuggauggaagccucacagauagcacaaacacgugcaaauuuuacacuagauagucucaaaauuuugacaccgguagcuacaucaacaaauuuaucacacagauuaaaggauacugcaacucagaugaaauucuccaguacaucauugaucagagucagcagauucauaacaauguccaaugauaacaugucuaucaaagaagcuaaugaaaccaaagauaccaaucuuauuuaucaacaaauaauguuaacaggauuaaguguuuucgaauauuuauuuagauuaaaagaaaccacaggacauaacccuauaguuaugcaucugcacauagaagaugaguguuguauuaaagaaaguuuuaaugaugaacauauuaauccagaaucuacauuagaauuaauucgauauccugaaaguaaugaauuuauuuaugauaaagacccacucaaagauguggacuuaucaaaacuuaugguuauuaaagaccauucuuacacaauugauaugaauuauugggaugauaccgauaucauacaugcaauuucaauauguacugcaauuacaauagcagauacuaugucacaauuagaucgagauaauuuaaaagagauaauagucauugcaaaugaugaugauauuaauagcuuaaucacugaauuuuugacucuugacauacuuguauuucuuaagacauuugguggauuauuaguaaaucaauuugcauacacucuuuauaguuuaaaaauagaagguagggaucucauuugggauuauauaaugagaacacugagagauacuucccauucaauauuaaaaguauuaucuaaugcauuaucucauccuaagguauucaagagguucugggauuguggaguuuuaaacccuauuuaugguccuaauacugcuagucaagaccagauaaaacuugcccuaucuauaugugaauauucacuagaucuauuuaugagagaaugguugaaugguguaucacuugaaauguacauuugugacagcgauauggaaguugcaaaugauaggaaacaagccuuuauuucuagacaccuuucauuuguuuguuguuuagcagaaauugcaucuuucggaccuaaccuguuaaacuuaacauacuuggagagacuugaucuauugaaacaauaucuugaauuaaauauuaaagaagacccuacucuuaaauauguacaaauaucuggauuauuaauuaaaucguucccaucaacuguaacauacguaagaaagacugcaaucaaauaucuaaggauucgugguauuaguccaccugaaguaauugaugauugggauccgguagaagaugaaaauaugcuggauaacauugucaaaacuauaaaugauaacugcaauaaagauaauaaagggaauaaaauuaacaauuucuggggacuagcacucaagaacuaucaaguccuuaaaaucagaucuauaacaagugauucugaugauaaugauagacuagaugcuaauacaagugguuugacacuuccccaaggagggaauuaucuaucgcaucaauugagauuauucggaaucaacagcacuaguugucugaaagcucuugaguuaucacaaauuuuaaugaaggaagucaauaaagacaaggacaggcucuuccugggagaaggagcaggagcuaugcuagcauguuaugaugccacauuaggaccugcaguuaauuauuauaauucagguuugaauauaacagauguaauuggucaacgagaauugaaaauauuuccuucagagguaucauuaguaggcaaaaaauuaggaaaugugacacagauucuuaacaggguaaaaguacuguucaaugggaaucccaauucaacauggauaggaaauauggaaugugagagcuugauauggagugaauuaaaugauaagucuauuggauuaguacauugugauauggaaggagccaucggcaaaucagaagaaacuguucuacaugaacauuauaguguuauaagaauuacauacuugauuggggaugaugauguuguuuuaguuuccaagauuauaccuacaaucacuccgaauuggucuagaauacuuuaucuauauaaauuauauuggaaagauguaaguauaauaucacucaaaacuucuaauccugcaucaacagaauuauaucuaauuucgaaagaugcauauuguacuauaauggaaccuagugaaauuguuuuaucaaaacuuaaaagauugucacucuuggaagaaaauaaucuauuaaaauggaucauuuuaucaaagaagaagaauaaugaaugguuacaucaugaaaucaaagaaggagaaagagauuauggagucaugagaccauaucauauggcacuacaaaucuuuggauuucaaaucaauuuaaaucaucuagcgaaagaauuuuuaucgaccccagaucugacuaauaucaacaauauaauccaaaguuuucagcggacaaucaaggauguuuuauuugaauggaucaauauaacucaugaugauaagagacacaaauuaggcgggagauauaacauauucccacugaaaaauaagggaaaguuaagacugcuaucaagaagacuaguauuaaguuggauuucauuaucauuaucgacucgauuacuuacaggucgcuuuccugaugaaaaauuugaacauagagcacagacaggauauguaucauuagcugauacugauuuagaaucauuaaaguuauugucgaaaaacgucauuaagaauuacagagaguguauaggaucaauaucauauugguuucuaacuaaggaaguuaaaauacuuaugaaauugauugguggugcuaaauuauuaggaauucccagacaauauaaagaacccgaagaucaguuauuagaaaacuacaaucaauaugaugaauuugauaucgauuaaaacauaaauacaaugaagauauauccuaaccuuuaucuuuaagccuaaggauagacaaaaaguaagaaaaacauguaauauauauauaccacacagagugcuucucuuguuuggu